(19) United States
(12) Patent Application Publication
(10) Pub. No.: US 2025/0064916 A1
(43) Pub. Date: Feb. 27, 2025

(54) IMMUNOGENIC COMPOSITIONS AND USES THEREOF

(71) Applicant: GLAXOSMITHKLINE BIOLOGICALS SA, Rixensart (BE)

(72) Inventors: Sylvie BERTHOLET GIRARDIN, Rockville, MD (US); Arun KUMAR, Siena (IT)

(73) Assignee: GLAXOSMITHKLINE BIOLOGICALS SA, Rixensart (BE)

(21) Appl. No.: 18/795,336

(22) Filed: Aug. 6, 2024

Related U.S. Application Data
(63) Continuation of application No. 17/268,647, filed on Feb. 16, 2021, now Pat. No. 12,083,174.
(60) Provisional application No. 62/719,219, filed on Aug. 17, 2018.

(51) Int. Cl. A61K 39/145; A61K 39/00

(52) U.S. Cl. CPC A61K 39/145; A61K 2039/53; A61K 2039/55555; A61K 2039/55566; A61K 2039/70

(57) ABSTRACT

The present invention is in the field of treating and/or preventing viral infections. In particular, the present invention relates to immunogenic or pharmaceutical compositions comprising self-replicating RNA molecules that encode influenza virus antigens for treating and/or preventing influenza infections.

### CROSS-REFERENCE TO RELATED APPLICATIONS

This is a Continuation of U.S. patent application Ser. No. 17/268,647, filed Feb. 16, 2021, which is a national stage entry of PCT/EP2019/072052, filed Aug. 16, 2019, which claims the benefit of U.S. Provisional No. 62/719,219 filed on Aug. 17, 2018. The entire contents of the foregoing applications are incorporated herein by reference.

### STATEMENT REGARDING SPONSORED RESEARCH

This invention was made with Government support under Agreement No. HR0011-12-3-0001 awarded by DARPA. The Government has certain rights in the invention. The work leading to this invention also received funding from the People Programme (Marie Curie Actions) of the European Union's Seventh Framework Programme (FP7/2007-2013) under REA grant agreement number 626283.

### SEQUENCE LISTING

The instant application contains a Sequence Listing which has been submitted electronically in XML file format and is hereby incorporated by reference in its entirety. Said XML copy, created on Aug. 8, 2024, is named VB66619C1-US_SL.xml and is 77,473 bytes in size.

### FIELD OF THE INVENTION

The present invention is in the field of treating and/or preventing viral infections. In particular, the present invention relates to immunogenic or pharmaceutical compositions comprising self-replicating RNA molecules that encode influenza virus antigens for treating and/or preventing influenza infections.

### BACKGROUND TO THE INVENTION

Influenza (flu) virus infections are liable for thousands of deaths annually worldwide and are responsible for an economic burden amounting to billions of dollars. Vaccines are the primary tool for prevention and control of the disease but as the flu virus frequently changes, the vaccines have to be reformulated every year. Available flu vaccines are strain-specific and provide protection against only vaccine strain viruses. Seasonal antigenic shifts resulting from frequent mutation of the viral surface proteins haemagglutinin (HA) and neuraminidase (NA) makes the previous season's vaccine largely ineffective in the following year. Further, the unpredictability of flu pandemics precipitated by antigenic shifts requires constant surveillance and significant investment in an attempt to maintain a level of preparedness. In view of these limitations, there is an urgent need for the development of novel flu vaccines that provide durable protection against multiple strains and subtypes, including future pandemic strains.

Live attenuated influenza vaccines (LAIV) and inactivated influenza vaccines (IIV) are presently used for vaccination in all age groups. Both vaccines have been found to induce homologous and heterologous immunity in human and in animals. However, adjuvanted subunit or split vaccines have been shown to induce strong HA-specific CD4 T-cell responses and high-titer HA-specific antibodies with a more diverse repertoire (Dormitzer et al Immunol Rev 2011; 239:167-77 and Khurana et al, Sci Transl Med 2011; 3:85ra48. Doi:10.1126/scitranslmed.3002336). The cumbersome production process for these vaccines involves the large-scale production of infectious virus in eggs and as a result antigenic domains of the vaccine viruses are altered. Therefore, new approaches need to be developed to avoid complex production processes that may alter the vaccine virus and the effectiveness of the resulting vaccine and to provide vaccines that show protection against multiple strains and/or subtypes of influenza virus.

### SUMMARY OF THE INVENTION

Provided herein in a first aspect is an immunogenic composition comprising: (i) a first self-replicating RNA molecule encoding a polypeptide comprising a first antigen and (ii) a second self-replicating RNA molecule encoding a polypeptide comprising a second antigen, wherein the first and second antigens are both from influenza virus but the first antigen is from a different strain of influenza virus to the second antigen.

In further aspects, is provided:
a pharmaceutical composition comprising an immunogenic composition described above and a pharmaceutically acceptable carrier,
the immunogenic composition or the pharmaceutical composition described above for use as a vaccine,
the immunogenic composition or the pharmaceutical composition described above for use in the prevention of influenza,
a method of prevention and/or treatment against influenza disease, comprising the administration of the immunogenic composition or pharmaceutical composition described above to a person in need thereof,
a method for inducing an immune response against influenza virus infection in a subject comprising administering to the subject an immunologically effective amount of the pharmaceutical composition or the immunogenic composition described above,
a method of prevention and/or treatment against influenza disease, comprising (i) the administration of a first immunogenic composition comprising a first self-replicating RNA molecule and pharmaceutically acceptable carrier and (ii) simultaneous or sequential administration of a second immunogenic composition comprising a second self-replicating RNA molecule and pharmaceutically acceptable carrier, wherein the first and second self-replicating RNA molecules encode a polypeptide encoding an antigen from influenza virus but the first self-replicating RNA molecule encodes an antigen from a different strain of influenza to that encoded by the second self-replicating RNA molecule,
a first immunogenic composition comprising a first self-replicating RNA molecule and a pharmaceutically acceptable carrier for use in a method of preventing influenza disease, said method comprising administration to a subject in need the first immunogenic composition followed by administration of a second immunogenic composition comprising a self-replicating RNA molecule and a pharmaceutically acceptable carrier, wherein the first and second self-replicating RNA molecules each encode a polypeptide comprising an antigen, wherein the antigen is from influenza virus but the antigen encoded by the first self-replicating RNA molecule is from a different strain of influenza virus to that encoded by the second self-replicating RNA molecule,
a method of preparing an immunogenic composition as described above, the method comprising: (i) providing at least one lipid which forms nanoparticles; (ii) providing an aqueous solution comprising the self-replicating RNA molecules; and (iii) combining the aqueous solution of (ii) and the at least one lipid of (i), thereby preparing the composition.
   a method of preparing an immunogenic composition as described above, the method comprising: (i) providing an oil-in-water emulsion; (ii) providing an aqueous solution comprising the self-replicating RNA molecules; and (iii) combining the aqueous solution of (ii) and the oil-in-water emulsion of (i), thereby preparing the composition.

### DESCRIPTION OF DRAWINGS/FIGURES

FIG. 1A-FIG. 1B: Schematic representation and characterization of bi-cistronic SAM (H5-H1). FIG. 1A demonstrates Bicistronic replicon construct used to test the multiple expression of HA genes. FIG. 1B demonstrates HA protein expression in BHK cells transfected with H1-SAM (Lane 1), H5-SAM (Lane 3), H5H1-SAM (Lane 4) or mock-treated (Lane 3). Cell lysates were analyzed by non-reducing SDS-PAGE followed by Western Blot analyses with HA-strain specific serum: i) Anti-A/California/07/2009 (H1N1) HA Serum (NIBSC, London, UK) used to detect H1; ii) Anti-A/turkey/Turkey/1/05 (H5N1) HA Serum (NIBSC, London, UK) used to detect H5. The monomeric form of HA is indicated by an asterisk (62 kDa). SGP=subgenomic promoter; H1=H1 HA gene from influenza A/California/07/2009 (H1N1) virus strain; H5=H5 HA gene from A/turkey/Turkey/01/2005 (H5N1) virus strain; 2A=full length 2A-driven sequence; nsPs=non-structural replicon proteins; HA*monomer.

FIG. 2A-FIG. 2M: Immunogenicity of SAM monocistronic and bicistronic SAM(HA)/LNP candidate vaccines. Mice (n=10) were immunized i.m. on day 0 and day 21 with SAM(H1), SAM(H5), SAM(H5-H1) and SAM(H1)+SAM(H5) at 0.1 μg. Sera and spleens were collected 2 weeks after second immunization. Sera samples were analyzed for A/California/07/2009 (H1N1) (demonstrated in FIG. 2A), A/turkey/Turkey/5/2005 (H5N1) (demonstrated in FIG. 2B), A/PR/8/1934 (H1N1) (demonstrated in FIG. 2C) and A/Perth/16/2009 (H3N2)-specific HI titres (demonstrated in FIG. 2D). Splenocytes (n=6) were stimulated in-vitro with H1-Cal (demonstrated in FIG. 2E, FIG. 2H, and FIG. 2K), H1-PR8 Cal (demonstrated in FIG. 2G, FIG. 2J, and FIG. 2M) and H5-turkey peptide pools Cal (demonstrated in FIG. 2F, FIG. 2I, and FIG. 2L), and T cells were analyzed for cytokine production by flow cytometry. The bars represent the cumulative frequency of H1-Cal, H5-turkey, H1-PR8-specific CD4⁺ T cells Cal (demonstrated in FIG. 2E, FIG. 2F, and FIG. 2G) and CD8⁺ T cells Cal (demonstrated in FIG. 2H, FIG. 2I, and FIG. 2J) expressing cytokines. CD107a expression by CD8 Cal (demonstrated in FIG. 2K, FIG. 2L, and FIG. 2M).

Statistical analyses were performed using the Mann-Whitney U test. **P<0.01, ***P<0.001.

FIG. 3A-FIG. 3L: Comparative immunogenicity of multivalent SAM(HA)/LNP candidate vaccines. Mice (n=10) were immunized i.m. on day 0 and day 21 with PBS [group 1] and combinations of SAM(H3-Biltho)+SAM(H3-Bang)+SAM(H3-Fuj) [group 2]; SAM(H3-Beij)+SAM(H3-Bris)+SAM(H3-Tex) [group 3]; SAM(H1-Cal)+SAM(H1-PR8)+SAM(H5-turkey)+SAM (H7-Shan) [group 4]; SAM(H3-Biltho)+SAM(H3-Bang)+SAM(H3-Fuj)+SAM(H3-Beij)+SAM(H3-Bris)+SAM(H3-Tex) [group 5] and SAM(H3-Biltho)+SAM(H3-Bang)+SAM(H3-Fuj)+SAM(H3-Beij)+SAM(H3-Bris)+SAM(H3-Tex)+SAM(H1-Cal)+SAM(H1-PR8)+SAM(H5-turkey)+SAM (H7-Shan) [group 6] Sera samples were collected 2 weeks after second immunization. Sera samples were analyzed for HA-specific HI titres with homologous Cal (demonstrated in FIG. 3A, FIG. 3B, FIG. 3C, FIG. 3D, FIG. 3E, FIG. 3F, FIG. 3G) and heterologous Cal (demonstrated in FIG. 3H, FIG. 3I, FIG. 3J, FIG. 3K, FIG. 3L) influenza virus antigens. Statistical analyses were performed using the Mann-Whitney U test. *P<0.05 **P<0.01, ***P<0.001, ns; not significant).

FIG. 4A-FIG. 4L: Comparative CD4⁺ T-cell responses induced by multivalent SAM(HA)/LNP candidate vaccines. Mice were immunized i.m. on day 0 and day 21 with PBS [group 1] and combinations of SAM(H3-Biltho)+SAM(H3-Bang)+SAM(H3-Fuj) [group 2]; SAM(H3-Beij)+SAM(H3-Bris)+SAM(H3-Tex) [group 3]; SAM(H1-Cal)+SAM(H1-PR8)+SAM(H5-turkey)+SAM (H7-Shan) [group 4]; SAM(H3-Biltho)+SAM(H3-Bang)+SAM(H3-Fuj)+SAM(H3-Beij)+SAM(H3-Bris)+SAM(H3-Tex) [group 5] and SAM(H3-Biltho)+SAM(H3-Bang)+SAM(H3-Fuj)+SAM(H3-Beij)+SAM(H3-Bris)+SAM(H3-Tex)+SAM(H1-Cal)+SAM(H1-PR8)+SAM(H5-turkey)+SAM (H7-Shan) [group 6]. Splenocytes (n=6) were stimulated in-vitro with HA peptide pools (demonstrated in FIG. 4D, FIG. 4 E, FIG. 4 F, and FIG. 4I), and recombinant HA proteins (demonstrated in FIG. 4A, FIG. 4B, FIG. 4C, FIG. 4G, FIG. 4H, FIG. 4J, FIG. 4K, and FIG. 4L). CD4⁺ T-cell mediated homologous (demonstrated in FIG. 4A, FIG. 4B, FIG. 4C, FIG. 4D, FIG. 4E, and FIG. 4F) and heterologous (demonstrated in FIG. 4G, FIG. 4H, FIG. 4I, FIG. 4J, FIG. 4K, FIG. 4L) responses were analyzed for cytokine production by flow cytometry. The bars represent the cumulative frequency of CD4⁺ T cells producing cytokines.

FIG. 5: a schematic diagram showing the immunization schedule used for Example 5.

FIG. 6: a graphical map of the TC83 vector containing an insert of H5 HA A/turkey/turkey at positions 7562-9256.

FIG. 7A-FIG. 7B: RNA quality and confirmation of HA H3 gene expression. (FIG. 7A) the SAM vectors encoding the H3 antigen were analyzed on a denaturing agarose gel. (FIG. 7B) SAM vectors encoding H3 antigens were transiently infected into BHK cells and cell lysates were subjected to SDS-PAGE (sodium dodecyl sulfate polyacrylamide gel electrophoresis) and finally blotted to nitrocellulose membranes. H3 expression was visualized by Western blotting using H3-specific polyclonal antibodies.

### DETAILED DESCRIPTION OF THE INVENTION

In the present study, by applying fully synthetic novel self-amplifying mRNA (SAM) vaccine technology, multivalent vaccine candidates were developed and immunogenicity tested in a Balb/c mice model. Vaccines were made comprising cocktails of two or more SAM vectors expressing full-length HAs from H3N2, H1N1, H5N1 and H7N9 subtypes either in monocistronic or bicistronic form and were encapsulated in lipid-nano particles (LNPs). It was demonstrated that in mice, SAM(H1), SAM(H5), SAM(H5-H1) or SAM(H1)+SAM(H5) induced CD4⁺ or CD8⁺ T-cell mediated heterologous responses, 3 weeks after the second immunization. However, this comparison of monocistronic and bicistronic SAM vectors suggested that combinations of two different influenza antigens in a single SAM vector is not as effective in boosting functional antibody responses as monocistronic SAM vectors. Cocktails of 3, 4, 6 or 10 monocistronic SAM(HA) vectors were prepared and were able to induce detectable cross-reactive B and T-cell responses in mice. Hence, for the first time, the inventors have shown a SAM(HA) multivalent vaccine that is able to induce broadly protective immune responses in Balb/c mice, against homologous and heterologous influenza viruses.

### Self Replicating RNA Molecule

Immunogenic compositions of the invention comprise self replicating RNA molecules, each self replicating RNA molecule encoding a polypeptide comprising an antigen.

Self-replicating RNA molecules are well known in the art and can be produced by using replication elements derived from, e.g. alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus the delivered RNA leads to the production of multiple daughter RNAs. These daughter RNAs, as well as collinear subgenomic transcripts, may be translated themselves to provide in situ expression of an encoded antigen (i.e. an influenza virus antigen), or may be transcribed to provide further transcripts with the same sense as the delivered RNA which are translated to provide in situ expression of the antigen. The overall result of this sequence of transcriptions is a huge amplification in the number of the introduced replicon RNAs and so the encoded antigen becomes a major polypeptide product of the cells.

One suitable system for achieving self-replication in this manner is to use an alphavirus-based replicon. These replicons are +-stranded (positive sense-stranded) RNAs which lead to translation of a replicase (or replicase-transcriptase) after delivery to a cell. The replicase is translated as a polyprotein which auto-cleaves to provide a replication complex which creates genomic-strand copies of the +-strand delivered RNA. These negative (−)-stranded transcripts can themselves be transcribed to give further copies of the +-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used e.g. the attenuated TC83 mutant of VEEV has been used in replicons, see the following reference: WO2005/113782, the context of which is incorporated by reference.

In one embodiment, each self-replicating RNA molecule described herein encodes (i) a RNA-dependent RNA polymerase which can transcribe RNA from the self-replicating RNA molecule and (ii) a polypeptide comprising an antigen from influenza virus. The polymerase can be an alphavirus replicase e.g. comprising one or more of alphavirus proteins nsP1, nsP2, nsP3 and nsP4.

Whereas natural alphavirus genomes encode structural virion proteins in addition to the non-structural replicase polyprotein, in certain embodiments, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the immunogen of interest, such that the subgenomic transcript encodes the immunogen rather than the structural alphavirus virion proteins.

Thus a self-replicating RNA molecule useful with the invention may have two open reading frames. The first (5′) open reading frame encodes a replicase; the second (3′) open reading frame encodes a polypeptide comprising an antigen from influenza virus. In some embodiments the RNA may have additional (e.g. downstream) open reading frames e.g. to encode further antigens or to encode accessory polypeptides.

In certain embodiments, the self-replicating RNA molecule disclosed herein has a 5′ cap (e.g. a 7-methylguanosine). This cap can enhance in vivo translation of the RNA. In some embodiments the 5′ sequence of the self-replicating RNA molecule must be selected to ensure compatibility with the encoded replicase.

A self-replicating RNA molecule may have a 3′ poly-A tail. It may also include a poly-A polymerase recognition sequence (e.g. AAUAAA) near its 3′ end.

Self-replicating RNA molecules can have various lengths but they are typically 5000-25000 nucleotides long. Self-replicating RNA molecules will typically be single-stranded. Single-stranded RNAs can generally initiate an adjuvant effect by binding to TLR7, TLR8, RNA helicases and/or PKR. RNA delivered in double-stranded form (dsRNA) can bind to TLR3, and this receptor can also be triggered by dsRNA which is formed either during replication of a single-stranded RNA or within the secondary structure of a single-stranded RNA.

The self-replicating RNA can conveniently be prepared by in vitro transcription (IVT). IVT can use a (cDNA) template created and propagated in plasmid form in bacteria, or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the self-replicating RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

A self-replicating RNA can include (in addition to any 5′ cap structure) one or more nucleotides having a modified nucleobase. A RNA used with the invention ideally includes only phosphodiester linkages between nucleosides, but in some embodiments it can contain phosphoramidate, phosphorothioate, and/or methylphosphonate linkages.

The self-replicating RNA molecule may encode a single heterologous polypeptide antigen (i.e. an antigen from influenza virus) or, optionally, two or more heterologous polypeptide antigens linked together in a way that each of the sequences retains its identity (e.g., linked in series) when expressed as an amino acid sequence. The heterologous polypeptides generated from the self-replicating RNA may then be produced as a fusion polypeptide or engineered in such a manner to result in separate polypeptide or peptide sequences. Self-replicating RNA molecules that encode a single heterologous polypeptide antigen may be termed “monocistronic” as described herein. Self-replicating RNA molecules that encode more than one, such as two separate heterologous polypeptide antigens, may be termed “multicistronic” or “bicistronic” respectively. Preferably, the self-replicating RNA molecules according to the invention are monocistronic.

The self-replicating RNA molecules described herein may be engineered to express multiple nucleotide sequences, from two or more open reading frames, thereby allowing co-expression of proteins, such as one, two or more influenza virus antigens, together with cytokines or other immunomodulators, which can enhance the generation of an immune response. Alternatively or in addition, the influenza virus antigen, e.g. an HA antigen, is the only antigen from influenza virus in each self-replicating RNA molecule.

If desired, the self-replicating RNA molecules can be screened or analyzed to confirm their therapeutic and prophylactic properties using various in vitro or in vivo testing methods that are known to those of skill in the art. For example, vaccines comprising self-replicating RNA molecule can be tested for their effect on induction of proliferation or effector function of the particular lymphocyte type of interest, e.g., B cells, T cells, T cell lines, and T cell clones. For example, spleen cells from immunized mice can be isolated and the capacity of cytotoxic T lymphocytes to lyse autologous target cells that contain a self-replicating RNA molecule that encodes an antigen from influenza virus. In addition, T helper cell differentiation can be analyzed by measuring proliferation or production of TH1 (IL-2 and IFN-γ) and/or TH2 (IL-4 and IL-5) cytokines by ELISA or directly in CD4+ T cells by cytoplasmic cytokine staining and flow cytometry.

Self-replicating RNA molecules that encode an antigen from influenza virus can also be tested for the ability to induce humoral immune responses, as evidenced, for example, by induction of B cell production of antibodies specific for an influenza virus antigen of interest. These assays can be conducted using, for example, peripheral B lymphocytes from immunized individuals. Such assay methods are known to those of skill in the art. Other assays that can be used to characterize the self-replicating RNA molecules can involve detecting expression of the encoded influenza virus antigen by the target cells. For example, FACS can be used to detect antigen expression on the cell surface or intracellularly. Another advantage of FACS selection is that one can sort for different levels of expression; sometimes-lower expression may be desired. Other suitable method for identifying cells which express a particular antigen involve panning using monoclonal antibodies on a plate or capture using magnetic beads coated with monoclonal antibodies.

In some embodiments, the self-replicating RNA molecule comprises a sequence that encodes (i) a RNA-dependent RNA polymerase which can transcribe RNA from the self-replicating RNA molecule and (ii) a polypeptide comprising an antigen from influenza virus. The polymerase can be an alphavirus replicase e.g. comprising one or more of alphavirus proteins nsP1, nsP2, nsP3 and nsP4.

In certain embodiments, immunogenic compositions according to the invention comprise self-replicating molecules wherein each self-replicating RNA molecule comprises a sequence that encodes (i) an alphavirus replicase which can transcribe RNA from the self-replicating RNA molecule and (ii) a polypeptide comprising hemagglutinin (HA) or an immunogenic fragment or variant thereof. Preferably, the polypeptide comprising HA or an immunogenic fragment or variant thereof is the only heterologous polypeptide encoded in the self-replicating RNA molecule. Typically, the HA or immunogenic fragment or variant thereof is the only antigen from influenza virus in the self-replicating RNA molecule.

For example, the self-replicating RNA molecules may comprise an RNA sequence encoded by the DNA sequence of SEQ ID NO: 2 or a DNA sequence that is at least 70%, at least 75%, at least 80%, at least 85%, at least 90%, at least 95%, at least 96%, at least 97%, at least 98%, or at least 99% identical to SEQ ID NO:2. In a particular embodiment, the self-replicating RNA molecules comprise an RNA sequence encoded by a DNA sequence at least 90% identical to SEQ ID NO:2. In some embodiments, the self replicating RNA molecules comprise an RNA sequence encoded by a fragment of a full-length sequence of SEQ ID NO:2 wherein the fragment comprises a contiguous stretch of the nucleic acid sequence of the full-length sequence up to 1, 2, 3, 4, 5, 6, 7, 8, 9, 10, 15, 20, 25, or 30 nucleic acids shorter than full-length sequence. In a specific embodiment, the self-relicating RNA molecules comprises an RNA sequence encoded by a fragment of SEQ ID NO:2 wherein the fragment comprises a contiguous stretch of the nucleic acid sequence of the full-length but up to 15 nucleic acids shorter than full-length sequence.

Immunogenic compositions as disclosed herein may comprise self-replicating molecules that each comprise an RNA sequence encoded by a DNA sequence that is at least 90% identical to SEQ ID NO:2, wherein the antigen from influenza virus encoded by each self-relicating RNA molecule is HA or an immunogenic fragment or variant thereof and wherein HA or the immunogenic fragment or variant thereof is the only antigen from influenza virus in each self-replicating RNA molecule. In such embodiments, the HA may be from the same subtype of influenza virus (intrasubtypic) or from a different subtype of influenza (heterosubtypic) for each self-replicating RNA molecule.

Polypeptide Comprising an Antigen from Influenza Virus

The self-relicating RNA molecules of the invention encode a polypeptide comprising an antigen from influenza virus. In certain embodiments, the antigen encoded is a wild type influenza virus polypeptide sequence, or is a fragment or variant thereof.

A “variant” of a polypeptide antigen sequence includes amino acid sequences having one or more amino acid substitutions, insertions and/or deletions when compared to the reference sequence. The variant may comprise an amino acid sequence which is at least 70%, at least 75%, at least 80%, at least 85%, at least 90%, at least 95%, at least 96%, at least 97%, at least 98%, or at least 99% identical to a full-length wild-type polypeptide, for example, to a polypeptide according to any one of SEQ ID NOs: 4, 6, 8, 10, 12, 14, 16, 18, 20 or 22. Alternatively, or in addition, a fragment of a polypeptide antigen may comprise an immunogenic fragment (i.e. an epitope-containing fragment) of the full-length polypeptide which may comprise a contiguous amino acid sequence of at least 8, at least 9, at least 10, at least 11, at least 12, at least 13, at least 14, at least 15, at least 16, at least 17, at least 18, at least 19, or more amino acids which is identical to a contiguous amino acid sequence of the full-length polypeptide.

A fragment of a polypeptide may comprise N- and/or C-terminal deletions when compared to a full-length polypeptide, wherein the fragment comprises a deletion of up to 1, 2, 3, 4, 5, 6, 7, 8, 9, 10, 15 or 20 amino acids from the N-terminus, the C-terminus, or both the N-terminus and C-terminus of the full-length sequence. It may be specified that the deletions are of consecutive amino acids.

As used herein, the term “antigen” refers to a molecule containing one or more epitopes (e.g., linear, conformational or both) that will stimulate a host's immune system to make a humoral and/or cellular antigen-specific immunological response (i.e. an immune response which specifically recognizes an antigen polypeptide). An “epitope” is that portion of an antigen that determines its immunological specificity.

Influenza viruses that infect humans can be classified into 3 types: A, B and C. A-type influenza viruses can be further classified into different subtypes, based on their HA (18 subtypes; H1, H2, H3, H4, H5, H6, H7, H8, H9, H10, H11, H12, H13, H14, H15, H16, H17 and H18) and NA proteins (11 subtypes, N1, N2, N3, N4, N5, N6, N7, N8, N9, N10 and N11). Influenza A viruses can be further broken down into different strains. B-type influenza viruses currently do not display different HA subtypes, but influenza B virus strains do fall into two distinct lineages. These lineages emerged in the late 1980s and have HAs which can be antigenically and/or genetically distinguished from each other. Current circulating influenza B virus strains belong to one of two lineages: B/Victoria or B/Yamagata. An internationally accepted naming convention for influenza viruses was accepted by WHO in 1979 and published in February 1980 in the Bulletin of the World Health Organization, 58(4):585-591 (1980) (“A revision of the system of nomenclature for influenza viruses: A WHO memorandum”). This approach uses a number of components in the naming system, including the antigenic type (A, B or C), geographical origin, year of isolation and strain number. Currently, influenza A(H1N1), A(H3N2) and one or two B strains are included in each year's influenza vaccine.

In the sense of the present invention, the term “influenza subtypes” is to be understood as A-type influenza virus strains having a given H subtype or a given N subtype, and the terms “different subtype” refer to influenza virus strains which do not share the same H subtype or the same N subtype. “Intrasubtypic” refers to the strains within the same influenza subtype, for example strains of a H1, H2, H3 etc HA subtype. “Heterosubtypic” refers to strains from different influenza subtypes, for example strains from the H1 subtype versus strains from the H3 subtype.

Influenza A viruses evolve and undergo antigenic variability continuously. A lack of effective proofreading by the viral RNA polymerase leads to a high rate of transcription errors that can result in amino-acid substitutions in surface glycoproteins, such as HA and NA proteins. This is termed “antigenic drift”. The segmented viral genome allows for a second type of antigenic variation. If two influenza viruses simultaneously infect a host cell, genetic reassortment, called “antigenic shift” may generate a novel virus with new surface or internal proteins. These antigenic changes, both ‘drifts’ and ‘shifts’ are unpredictable and may have a dramatic impact from an immunological point of view as they eventually lead to the emergence of new influenza virus strains and that enable the virus to escape the immune system causing the well known, almost annual, epidemics. Both of these genetic modifications have caused new viral variants responsible for pandemic in humans.

The self-replicating RNA molecules of the present invention may encode an influenza antigen from any type (A-type, B-type, C-type) and any subtype (H1 to H18 and N1 to N11) of influenza viruses, or immunogenic fragments or variants thereof. By way of example, the self-replicating RNA molecules as described herein may encode a polypeptide encoding an antigen from influenza virus wherein the antigen from influenza virus comprises the polypeptide sequence of any one of SEQ ID NOs: 4, 6, 8, 10, 12, 14, 16, 18, 20 or 22 or an immunogenic fragment or variant thereof. In a specific embodiment, the antigen from influenza virus comprises the sequence of any one of SEQ ID NOs: 4, 6, 8, 10, 12, 14, 16, 18, 20 or 22. Alternatively, the antigen from influenza virus comprises or consists of a variant with an amino acid sequence at least 90% identical to the sequence of any one of SEQ ID NOs: 4, 6, 8, 10, 12, 14, 16, 18, 20 or 22. Alternatively or in addition, the antigen from influenza virus comprises or consists of an immunogenic fragment comprising a deletion of up to 20 amino acids from the N-terminus, the C-terminus, or both the N-terminus and C-terminus of the full-length sequence of any one of SEQ ID NOs: 4, 6, 8, 10, 12, 14, 16, 18, 20 or 22.

Suitably, the influenza virus antigen encoded by a self-replicating RNA molecule according to the invention is from a pandemic strain. By pandemic strain, is meant a new influenza virus against which the large majority of the human population has no immunity. Throughout the document “a pandemic strain” will refer to an influenza virus strain being associated with or with the potential to be associated with an outbreak of pandemic influenza disease, such as pandemic Influenza A-type virus strains. A strain with the potential to be associated with a pandemic outbreak may comprise a strain (e.g. an animal influenza virus strain) that has infected humans and that is not a circulating seasonal strain of influenza in humans (e.g. not a strain of influenza A(H1N1) or A(H3N2) or B strains of either Victoria or Yamagata lineage). Pandemic strains associated with a pandemic outbreak or with the potential to be associated with a pandemic will be known to those skilled in the art and are identified by the WHO according to internationally recognised criteria (see “Pandemic influenza preparedness and response” A WHO Guidance document, 2009, IBSN 9789241547680).

Suitable pandemic strains are, H1, H2, H5, H6, H7 or H9 subtype influenza A virus strains, e.g. H5N1, H5N3, H2N2, H6N1, H9N2, H7N7, H2N2, H7N1, H7N9 and H1N1. Within the H5 subtype, a virus may fall into different clades. Other suitable pandemic strains in human are H7N3, H10N7 and H5N2. Alternatively, the influenza virus antigen may be from a circulating seasonal strain, i.e. a non-pandemic strain.

In certain embodiments, the antigen from influenza virus is hemagglutinin (HA) or neuraminidase (NA) or any other antigen such as Matrix protein 2 (M2), Matrix protein 1 (M1), nucleoprotein (NP), PB1 or PB2 or an immunogenic fragment or variant thereof. Preferably, the antigen is HA or an immunogenic fragment or variant thereof.

Polypeptides according to the invention may comprise, in addition to the antigen from influenza virus, one or more heterologous amino acid sequences (e.g. another antigen sequence, another signal sequence, a detectable tag, or the like). For example, the polypeptide herein may be a fusion protein.

### Nucleic Acid and its Preparation

Disclosed herein are nucleic acid molecules, such as DNA or RNA, comprising a sequence which encodes a polypeptide comprising an antigen from influenza virus. In the immunogenic compositions according to the invention, the self-replicating RNA molecules comprise such sequences in RNA form. Also disclosed herein, is nucleic acid in the form of self-relicating RNA molecules, with or without heterologous sequence encoding a polypeptide comprising an antigen from influenza virus.

Nucleic acid may be prepared in many ways, e.g. by chemical synthesis in whole or in part, by digesting longer nucleic acids using nucleases (e.g. restriction enzymes), by joining shorter nucleic acids or nucleotides (e.g. using ligases or polymerases), from genomic or cDNA libraries etc. For example, cDNA templates encoding a polypeptide comprising an antigen from influenza virus may be prepared. Such cDNA templates may be created and propagated in plasmid form in bacteria or created synthetically (for example by gene synthesis and/or polymerase chain-reaction (PCR) engineering methods). RNA sequences encoding polypeptides of the invention may be prepared for example, by in vitro transcription (IVT), which can use a DNA-dependent RNA polymerase (such as the bacteriophage T7, T3 or SP6 RNA polymerases) to transcribe RNA from the cDNA template.

The term “nucleic acid” in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the nucleic acid of the disclosure includes mRNA, self-replicating RNA, DNA, cDNA, recombinant nucleic acids, branched nucleic acids, plasmids, vectors, etc. Where the nucleic acid takes the form of RNA, it may or may not have a 5′ cap.

The nucleic acids herein comprise a sequence which encodes a polypeptide comprising an antigen from influenza virus. Typically, the nucleic acids of the invention will be in recombinant form, i.e. a form which does not occur in nature. For example, the nucleic acid may comprise one or more heterologous nucleic acid sequences (e.g. a sequence encoding another antigen and/or a control sequence such as a promoter or an internal ribosome entry site) in addition to the sequence encoding an antigen from influenza virus. The nucleic acid may be part of a vector i.e. part of a nucleic acid construct designed for transduction/transfection of one or more cell types. Vectors may be, for example, “expression vectors” which are designed for expression of a nucleotide sequence in a host cell, or “viral vectors” which are designed to result in the production of a recombinant virus or virus-like particle.

The nucleic acid encoding the polypeptides described above may be codon optimized. By “codon optimized” is intended modification with respect to codon usage that may increase translation efficacy and/or half-life of the nucleic acid. A poly A tail (e.g., of about 30 adenosine residues or more) may be attached to the 3′ end of the RNA to increase its half-life. The 5′ end of the RNA may be capped with a modified ribonucleotide with the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methytransferase, which catalyzes the construction of N7-monomethylated cap 0 structures). Cap 0 structure plays an important role in maintaining the stability and translational efficacy of the RNA molecule. The 5′ cap of the RNA molecule may be further modified by a 2′-O-Methyltransferase which results in the generation of a cap 1 structure (m7Gppp [m2′-O]N), which may further increases translation efficacy.

The nucleic acids may comprise one or more nucleotide analogs or modified nucleotides. As used herein, “nucleotide analog” or “modified nucleotide” refers to a nucleotide that contains one or more chemical modifications (e.g., substitutions) in or on the nitrogenous base of the nucleoside (e.g. cytosine (C), thymine (T) or uracil (U)), adenine (A) or guanine (G)). A nucleotide analog can contain further chemical modifications in or on the sugar moiety of the nucleoside (e.g., ribose, deoxyribose, modified ribose, modified deoxyribose, six-membered sugar analog, or open-chain sugar analog), or the phosphate. The preparation of nucleotides and modified nucleotides and nucleosides are well-known in the art, see the following references: U.S. Pat. Nos. 4,373,071, 4,458,066, 4,500,707, 4,668,777, 4,973,679, 5,047,524, 5,132,418, 5,153,319, 5,262,530, 5,700,642. Many modified nucleosides and modified nucleotides are commercially available.

Modified nucleobases which can be incorporated into modified nucleosides and nucleotides and be present in the RNA molecules include: m5C (5-methylcytidine), m5U (5-methyluridine), m6A (N6-methyladenosine), s2U (2-thiouridine), Um (2′-O-methyluridine), mIA (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6-isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); mil (1-methylinosine); m′Im (1,2′-O-dimethylinosine); m3C (3-methylcytidine); Cm (2T-O-methylcytidine); s2C (2-thiocytidine); ac4C (N4-acetylcytidine); 5FC (5-fonnylcytidine); m5Cm (5,2-O-dimethylcytidine); ac4Cm (N4acetyl2TOmethylcytidine); k2C (lysidine); mIG (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethy 1 aminomethyl-2-L-Omethyl uridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,T-O-dimethyladenosine); rn62Am (N6,N6,0-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); F5Cm (5-formyl-2′-O-methylcytidine); mlGm (1,2′-O-dimethylguanosine); m′Am (1,2-O-dimethyl adenosine) irinomethyluridine); tm5s2U (S-taurinomethyl-2-thiouridine)); iniG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(Ci-Ce)-alkyluracil, 5-methyluracil, 5-(C2-C6)-alkenyluracil, 5-(C2-Ce)-alkynyluracil, 5-(hydroxymethyl)uracil, 5-chlorouracil, 5-fluorouracil, 5-bromouracil, 5-hydroxycytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Many of these modified nucleobases and their corresponding ribonucleosides are available from commercial suppliers.

### Immunogenic and Pharmaceutical Compositions

Immunogenic compositions according the invention comprise self-replicating RNA molecules that encode polypeptides comprising an antigen from influenza virus. Such compositions may be a vaccine, in particular an RNA based vaccine.

Immunogenic compositions according to the invention comprise a first self-replicating RNA molecule encoding a polypeptide comprising a first antigen and a second self-replicating RNA molecule encoding a polypeptide comprising a second antigen. In certain embodiments, the immunogenic composition according to the invention comprises or contains further self-replicating molecules, such as 3, 4, 5, 6, 7, 8, 9 or 10 self-replicating RNA molecules. In particular, the immunogenic composition may comprise a third self-replicating RNA molecule encoding a polypeptide comprising a third antigen from influenza virus and optionally a fourth self-replicating RNA molecule encoding a polypeptide comprising a fourth antigen from influenza virus. Yet further self-replicating RNA molecules may be present in the immunogenic composition of the invention, for example:
a 5^{th} self-replicating RNA molecule encoding a polypeptide comprising a 5^{th} antigen from influenza virus and optionally
a 6th self-replicating RNA molecule encoding a polypeptide comprising a 6th antigen from influenza virus and optionally
a 7th self-replicating RNA molecule encoding a polypeptide comprising a 7th antigen from influenza virus and optionally
an 8th self-replicating RNA molecule encoding a polypeptide comprising an 8th antigen from influenza virus and optionally
a 9th self-replicating RNA molecule encoding a polypeptide comprising a 9th antigen from influenza virus and optionally
a 10th self-replicating RNA molecule encoding a polypeptide comprising a 10th antigen from influenza virus.

In these embodiments, the first, second, third and/or optional subsequent antigens from influenza virus are all from different strains of influenza virus. In some embodiments, the antigen in each self-replicating RNA molecule may be from a different subtype of influenza virus as compared to the remaining antigen/s (e.g. for HA; H1, H2, H3, H5, H7, H9 etc). In certain embodiments, the immunogenic compositions with 2, 3, 4, 5, 6, 7, 8, 9 or 10 self-replicating molecules contain only these self-replicating RNA molecules.

Typically, the immunogenic composition comprises or contains up to 10 self-replicating RNA molecules, such as from 2 to 10 self-replicating RNA molecules, from 2 to 8 self-replicating RNA molecules, from 3 to 7 self-replicating molecules, from 3 to 6 self-replicating RNA molecules or from 4 to 6 self-replicating RNA molecules, wherein each self-replicating RNA molecule encodes a polypeptide comprising an antigen from influenza virus and wherein each antigen is from a different strain of influenza virus. In a particular embodiment, the immunogenic composition according to the invention contains from 3 to 10 self-replicating RNA molecules.

In one embodiment, the first antigen and/or second antigen and/or optional subsequent antigens in the immunogenic compositions of the invention is hemagglutin (HA). Typically, the HA antigen may be derived from an influenza virus of a past or present seasonal or pandemic strain. For example, HA may be selected from seasonal strains of type H1 or H3, or pandemic strains of type H1, H2, H5, H6, H7, H9 or H10, such as H5 or H7. In one embodiment, hemagglutinin is the only antigen from influenza virus in the immunogenic composition.

In certain embodiments, the immunogenic composition comprises a first and/or second antigen that is HA from influenza virus H1. In other embodiments, the immunogenic composition comprises a first and/or second antigen that is HA from influenza virus H3. In a further embodiment, the first antigen is from influenza virus H1 and the second antigen is from influenza virus H3. In particular, the H1 influenza virus strain may be from A/H1N1 and the H3 influenza virus strain may be an A/H3N2 strain.

Other combinations of antigens from influenza virus are envisaged. E.g. for an immunogenic composition according to the invention which comprises two self-replicating molecules, the first and second antigens may be HA derived from the following hemagglutinin strain types respectively: H1+H1, H3+H3, H1+H3, H5+H7, H5+H5 or H7+H7. For an immunogenic composition according to the invention which comprises three self-replicating molecules, the first, second and third antigens may be derived from the following hemagglutinin strain types respectively: H1+H1+H1, H1+H1+H3, H1+H3+H3, H3+H3+H3, H1+H3+H5, H3+H3+H5, H3+H3+H7 or H1+H5+H7. For an immunogenic composition according to the invention which comprises four self-replicating molecules, the first, second, third and further antigens may be HA derived from the following strain types respectively: H1+H1+H3+H3, H1+H3+H3+H3, H1+H3+H3+H5, H1+H3+H5+H7 or H3+H3+H3+H3.

In any of these embodiment comprising two, three or four self replicating molecules, the HA antigens may be derived from a A/H1N1 strain. In addition, the HA antigens may be derived from a A/H3N2 strain.

Hence, in one embodiment, an immunogenic composition of the invention comprises (i) a first self-replicating RNA molecule encoding a polypeptide comprising a first antigen and (ii) a second self-replicating RNA molecule encoding a polypeptide comprising a second antigen, wherein:
the first and second antigens are both from influenza virus but the first antigen is from a different strain of influenza virus to the second antigen,
the first and second antigens are hemagglutinin or an immunogenic fragment or variant thereof,
the first and second antigens are from strains of influenza virus with a different geographical origin and/or year of isolation.

In a further embodiment, an immunogenic composition of the invention comprises (i) a first self-replicating RNA molecule encoding a polypeptide comprising a first antigen and (ii) a second self-replicating RNA molecule encoding a polypeptide comprising a second antigen, wherein:
the first and second antigens are both from influenza virus but the first antigen is from a different strain of influenza virus to the second antigen,
the first and second antigen is hemagglutinin or an immunogenic fragment or variant thereof and
the first and second antigens are from influenza viruses with a different hemagglutinin subtype.

In a further embodiment, an immunogenic composition of the invention comprises (i) a first self-replicating RNA molecule encoding a polypeptide comprising a first antigen and (ii) a second self-replicating RNA molecule encoding a polypeptide comprising a second antigen, wherein:
the first and second antigens are both from influenza virus but the first antigen is from a different strain of influenza virus to the second antigen,
the first and second antigen is hemagglutinin or an immunogenic fragment or variant thereof and
the first antigen is from a seasonal influenza virus (e.g. a H1N1, H3N2, B/Victoria or B/Yamagata strain) and the second antigen is from a pandemic influenza virus strain.

In a further embodiment, an immunogenic composition of the invention comprises (i) a first self-replicating RNA molecule encoding a polypeptide comprising a first antigen (ii) a second self-replicating RNA molecule encoding a polypeptide comprising a second antigen, and (iii) a third self-replicating RNA molecule encoding a polypeptide comprising a third antigen, wherein:
the first, second and third antigens are both from influenza virus but the first, second and third antigens are all from different strains of influenza virus,
the first, second and third antigens are hemagglutinin or an immunogenic fragment or variant thereof and
the first, second and third antigens are all from influenza viruses in the same hemagglutinin subtype but from viruses with a different geographical origin and/or year of isolation.

In a further embodiment, an immunogenic composition of the invention comprises (i) a first self-replicating RNA molecule encoding a polypeptide comprising a first antigen (ii) a second self-replicating RNA molecule encoding a polypeptide comprising a second antigen, and (iii) a third self-replicating RNA molecule encoding a polypeptide comprising a third antigen, wherein:
the first, second and third antigens are both from influenza virus but the first, second and third antigens are all from different strains of influenza virus,
the first, second and third antigens are hemagglutinin or an immunogenic fragment or variant thereof and
the first and second antigens are from past or present seasonal influenza viruses and the third antigen is from a pandemic influenza virus.

In one embodiment, the immunogenic composition may comprise multiple self-replicating RNA molecules, where each self-replicating RNA molecule encodes a polypeptide comprising an HA antigen from influenza virus H3. For example, the composition may comprise 2, 3, 4, 5, 6, 7, 8, 9 or 10 self-replicating RNA molecules encoding a polypeptide comprising an HA antigen from influenza virus H3.

In some such embodiments, the HA antigens are selected from strains of the influenza H3N2 subtype. For example, in one embodiment, HA antigens are selected from potential pandemic and seasonal strains of the influenza H3N2 subtypes. These strains of the H3N2 subtype may be selected on the basis of the following criteria: 1) major glycosylation sites, 2) important seasonal and pandemic vaccine strains, and 3) cell adapted strains only.

In one embodiment, the immunogenic composition comprises multiple self-replicating RNA molecules, where each self-replicating RNA molecule encodes a polypeptide comprising an HA antigen from a different strain of the H3N2 influenza virus.

In one embodiment, the immunogenic composition comprises three self-replicating RNA molecules, wherein: (i) a first self-replicating RNA molecule encodes a polypeptide comprising a first antigen from A/Bilthoven/16398/1968 (EPI362379), (ii) a second self-replicating RNA molecule encodes a polypeptide comprising a second antigen from A/Bangkok/1/79 (EPI367158), (iii) a third self-replicating RNA molecule encodes a polypeptide comprising a third antigen from A/Fujian/411/2002 (EPI362915).

In another embodiment, the immunogenic composition comprises three self-replicating RNA molecules, wherein: (i) a first self-replicating RNA molecule encodes a polypeptide comprising a first antigen from A/Beijing/32/92 (EPI365898), (ii) a second self-replicating RNA molecule encodes a polypeptide comprising a second antigen from A/Brisbane/10/2007 (EPI362338), and (iii) a third self-replicating RNA molecule encodes a polypeptide comprising a third antigen from A/Texas/50/2012 (EPI398417).

In one preferred embodiment, the immunogenic composition comprises six self-replicating RNA molecules, wherein: (i) a first self-replicating RNA molecule encodes a polypeptide comprising a first antigen from A/Bilthoven/16398/1968 (EPI362379), (ii) a second self-replicating RNA molecule encodes a polypeptide comprising a second antigen from A/Bangkok/1/79 (EPI367158), (iii) a third self-replicating RNA molecule encodes a polypeptide comprising a third antigen from A/Beijing/32/92 (EPI365898), (iv) a fourth self-replicating RNA molecule encodes a polypeptide comprising a fourth antigen from A/Fujian/411/2002 (EPI362915), (v) a fifth self-replicating RNA molecule encodes a polypeptide comprising a fifth antigen from A/Brisbane/10/2007 (EPI362338), and (vi) a sixth self-replicating RNA molecule encodes a polypeptide comprising a sixth antigen from A/Texas/50/2012 (EPI398417). Sequence information for full length genes encoding for influenza HA proteins was obtained from the Global Initiative on Sharing All Influenza Data Epiflu database (www.gisaid.org).

The immunogenic composition of all of these embodiments may also include one or more further self-replicating RNA molecules, where one or more further self-replicating RNA molecules encode a polypeptide comprising an HA antigen from influenza virus H1, H5 and/or H7. In such embodiments, the self-replicating RNA molecules may encode polypeptides comprising an HA antigen from two different influenza H1 viruses. Additionally, the self-replicating RNA molecules may encode a polypeptide comprising an HA antigen from influenena subtype H5. The self-replicating RNA molecules may also encode a polypeptide comprising an HA antigen from influenena subtype H7.

In one embodiment, the immunogenic composition comprises ten self-replicating RNA molecules, wherein: (i) a first self-replicating RNA molecule encodes a polypeptide comprising a first antigen from A/Bilthoven/16398/1968 (EPI362379), (ii) a second self-replicating RNA molecule encodes a polypeptide comprising a second antigen from A/Bangkok/1/79 (EPI367158), (iii) a third self-replicating RNA molecule encodes a polypeptide comprising a third antigen from A/Beijing/32/92 (EPI365898), (iv) a fourth self-replicating RNA molecule encodes a polypeptide comprising a fourth antigen from A/Fujian/411/2002 (EPI362915), (v) a fifth self-replicating RNA molecule encodes a polypeptide comprising a fifth antigen from A/Brisbane/10/2007 (EPI362338), and (vi) a sixth self-replicating RNA molecule encodes a polypeptide comprising a sixth antigen from A/Texas/50/2012 (EPI398417), (vii) a seventh self-replicating RNA molecule encodes a polypeptide comprising a seventh antigen from A/California/07/2009 (H1N1), (viii) an eighth self-replicating RNA molecule encodes a polypeptide comprising a eighth antigen from A/PR/8/1934 (H1N1), (ix) a ninth self-replicating RNA molecule encodes a polypeptide comprising a ninth antigen from A/turkey/Turkey/5/2005 (H5N1), and (x) a tenth self-replicating RNA molecule encodes a polypeptide comprising a tenth antigen from A/Shanghai/1/3013 (H7N9).

The immunogenic composition may comprise a viral or a non-viral delivery system. The delivery system (also referred to herein as a delivery vehicle) may have adjuvant effects which enhance the immunogenicity of the encoded antigen from influenza virus. For example, the self-replicating RNA molecules may be encapsulated in liposomes, non-toxic biodegradable polymeric microparticles or viral replicon particles (VRPs), or complexed with particles of a cationic oil-in-water emulsion. In some embodiments, the nucleic acid-based vaccine comprises a cationic nano-emulsion (CNE) delivery system or a lipid nanoparticle (LNP) delivery system. In some embodiments, the nucleic acid-based vaccine comprises a non-viral delivery system, i.e., the nucleic acid-based vaccine is substantially free of viral capsid. Alternatively, the nucleic acid-based vaccine may comprise viral replicon particles. In other embodiments, the nucleic acid-based vaccine may comprise a naked nucleic acid, such as naked RNA (e.g. mRNA), but delivery via CNEs or LNPs is preferred.

In certain embodiments, the nucleic acid-based vaccine comprises a cationic nano-emulsion (CNE) delivery system. CNE delivery systems and methods for their preparation are described in the following reference: WO2012/006380. In a CNE delivery system, the nucleic acid molecule (e.g. RNA) which encodes the antigen is complexed with a particle of a cationic oil-in-water emulsion. Cationic oil-in-water emulsions can be used to deliver negatively charged molecules, such as an RNA molecule to cells. The emulsion particles comprise an oil core and a cationic lipid. The cationic lipid can interact with the negatively charged molecule thereby anchoring the molecule to the emulsion particles. Further details of useful CNEs can be found in the following references: WO2012/006380; WO2013/006834; and WO2013/006837 (the contents of each of which are incorporated herein in their entirety).

Thus, in certain embodiments, in immunogenic compositions according to the invention, the self-replicating RNA molecules encoding a polypeptide comprising an antigen from influenza virus are complexed with a particle of a cationic oil-in-water emulsion. The particles typically comprise an oil core (e.g. a plant oil or squalene) that is in liquid phase at 25° C., a cationic lipid (e.g. phospholipid) and, optionally, a surfactant (e.g. sorbitan trioleate, polysorbate 80); polyethylene glycol can also be included. In some embodiments, the CNE comprises squalene and a cationic lipid, such as 1,2-dioleoyloxy-3-(trimethylammonio)propane (DOTAP). In some preferred embodiments, the delivery system is a non-viral delivery system, such as CNE. This may be particularly effective in eliciting humoral and cellular immune responses. Advantages also include the absence of a limiting anti-vector immune response and a lack of risk of genomic integration.

In some embodiments, self-replicating RNA molecules according to the invention may be complexed with a submicron cationic oil-in-water emulsion. In some embodiments the cationic oil-in-water emulsion is characterized by an average particle size of from about 80 nm to 180 nm in diameter (or alternatively from about 80 to about 150 nm; from about 80 to 130 nm; or from about 100 nm). In some embodiments, the concentration of DOTAP in said emulsion, before RNA complexation, is at least about 2.5 mM, or from about 2.5 mM to about 8 mM. In a particular embodiment, the concentration of DOTAP in said emulsion is about 4 mg/ml (5.73 mM). The oil can be squalene or squalane.

In some embodiments, in immunogenic compositions of the invention self-relicating RNA molecules are complexed to a cationic oil-in-water emulsion comprising DOTAP, squalene, sorbitan trioleate and polysorbate 80 in citrate buffer. Cationic oil-in-water emulsions suitable for delivery of an RNA molecule encoding a polypeptide comprising an antigen from influenza virus may contain about 2 mg/ml to 7 mg/ml DOTAP; about 3 mg/ml to 6 mg/ml Span 85; about 3 mg/ml to 6 mg/ml Tween 80; and about 30 mg/ml to 50 mg/ml squalene. In certain embodiments, the cationic oil-in-water emulsion, before complexing with RNA, contains about 4.3% w/v squalene, 0.5% Tween 80, 0.5% SPAN85, and 4 mg/mL DOTAP.

Hence, in one embodiment, an immunogenic composition of the invention comprises (i) a first self-replicating RNA molecule encoding a polypeptide comprising a first antigen and (ii) a second self-replicating RNA molecule encoding a polypeptide comprising a second antigen, wherein:
the first and second antigens are both from influenza virus but the first antigen is from a different strain of influenza virus to the second antigen,
the first and second antigens are hemagglutinin or an immunogenic fragment or variant thereof,
the self-replicating RNA molecules are formulated in lipid nanoparticles (LNP).

Hence, in one embodiment, an immunogenic composition of the invention comprises (i) a first self-replicating RNA molecule encoding a polypeptide comprising a first antigen and (ii) a second self-replicating RNA molecule encoding a polypeptide comprising a second antigen, wherein:
the first and second antigens are both from influenza virus but the first antigen is from a different strain of influenza virus to the second antigen,
the first and second antigens are hemagglutinin or an immunogenic fragment or variant thereof,
the self-replicating RNA molecules are formulated in a cationic nanoemulsion (CNE).

Hence, in one embodiment, an immunogenic composition of the invention comprises (i) a first self-replicating RNA molecule encoding a polypeptide comprising a first antigen and (ii) a second self-replicating RNA molecule encoding a polypeptide comprising a second antigen, wherein:
the first and second antigens are both from influenza virus but the first antigen is from a different strain of influenza virus to the second antigen,
the first and second antigens are hemagglutinin or an immunogenic fragment or variant thereof,
the self-replicating RNA molecules are formulated in lipid nanoparticles (LNP) or a cationic nanoemulsion (CNE) and
as well as encoding a polypeptide comprising an antigen from influenza virus, each self-replicating RNA molecule encodes a RNA-dependent RNA polymerase which can transcribe RNA from the self-replicating RNA molecule.

In a further embodiment, an immunogenic composition of the invention comprises (i) a first self-replicating RNA molecule encoding a polypeptide comprising a first antigen (ii) a second self-replicating RNA molecule encoding a polypeptide comprising a second antigen, and (iii) a third self-replicating RNA molecule encoding a polypeptide comprising a third antigen, wherein:
the first, second and third antigens are both from influenza virus but the first, second and third antigens are all from different strains of influenza virus,
the first, second and third antigens are hemagglutinin or an immunogenic fragment or variant thereof and
the self-replicating RNA molecules are formulated in lipid nanoparticles (LNP) or a cationic nanoemulsion (CNE).

In a further embodiment, an immunogenic composition of the invention comprises (i) a first self-replicating RNA molecule encoding a polypeptide comprising a first antigen (ii) a second self-replicating RNA molecule encoding a polypeptide comprising a second antigen, and (iii) a third self-replicating RNA molecule encoding a polypeptide comprising a third antigen, wherein:
the first, second and third antigens are both from influenza virus but the first, second and third antigens are all from different strains of influenza virus,
the first, second and third antigens are hemagglutinin or an immunogenic fragment or variant thereof,
the self-replicating RNA molecules are formulated in lipid nanoparticles (LNP) or a cationic nanoemulsion (CNE) and
as well as encoding a polypeptide comprising an antigen from influenza virus, each self-replicating RNA molecule encodes a RNA-dependent RNA polymerase which can transcribe RNA from the self-replicating RNA molecule.

Also provided is a method of preparing an immunogenic composition according to the invention wherein the self-replicating RNA molecules are complexed to a cationic oil-in-water emulsion, the method comprising: (i) providing an oil-in-water emulsion as described herein; (ii) providing an aqueous solution comprising the self-replicating RNA molecules; and (iii) combining the aqueous solution of (ii) and the oil-in-water emulsion of (i), thereby preparing the immunogenic composition. If desired, the aqueous solution comprising the RNA molecule may be a buffer. The buffer may comprise one or more salt, buffer, saccharide, or polymer. In an preferred embodiment, the buffer comprises 560 mM sucrose, 20 mM NaCl, and 10 mM citrate, which can be mixed with a cationic oil in water emulsion described herein to produce a final aqueous phase that comprises 280 mM sucrose, 10 mM NaCl and 10 mM citrate.

Also provided is a method of preparing an immunogenic composition according to the invention wherein the self-replicating RNA molecules are encapsulated in a lipid nanoparticle (LNP), the method comprising: (i) providing at least one lipid which forms nanoparticles; (ii) providing an aqueous solution comprising the self-replicating RNA molecules; and (iii) combining the aqueous solution of (ii) and the at least one lipid of (i), thereby preparing the composition.

LNP delivery systems and non-toxic biodegradable polymeric microparticles, and methods for their preparation are described in the following references: WO2012/006376 (LNP and microparticle delivery systems); Geall et al. (2012) PNAS USA. September 4; 109(36): 14604-9 (LNP delivery system); and WO2012/006359 (microparticle delivery systems). LNPs are non-virion liposome particles in which a nucleic acid molecule (e.g. RNA) can be encapsulated. The particles can include some external RNA (e.g. on the surface of the particles), but at least half of the RNA (and ideally all of it) is encapsulated. Liposomal particles can, for example, be formed of a mixture of zwitterionic, cationic and anionic lipids which can be saturated or unsaturated, for example; DSPC (zwitterionic, saturated), DlinDMA (cationic, unsaturated), and/or DMG (anionic, saturated). In some embodiments, the LNP comprises neutral lipids, cationic lipids, cholesterol and polyethylene glycol (PEG) and forms nanoparticles that encompass the self-amplifying RNA. Preferred LNPs for use with the invention include an amphiphilic lipid (helper lipid(s)) which can form liposomes, optionally in combination with at least one cationic lipid (such as DOTAP, DSDMA, DODMA, DLinDMA, DLenDMA, etc.). Useful helper lipids include zwitterionic lipids, such as DPPC, DOPC, DSPC, dodecylphosphocholine, 1,2-dioleoyl-sn-glycero-3-phosphatidylethanolamine (DOPE), and 1,2-diphytanoyl-sn-glycero-3-phosphoethanolamine (DPyPE); sterols, such as cholesterol; and PEGylated lipids, such as PEG-DMPE (PEG-conjugated 1,2-dimyristoyl-Sn-glycero-3-phosphoethanolamine-N-[methoxy (polyethylene glycol)]) or PEG-DMG (PEG-conjugated 1,2-Dimyristoyl-sn-glycerol, methoxypolyethylene Glycol). In some embodiments, useful PEGylated lipids may be PEG2K-DMPE (PEG-conjugated 1,2-dimyristoyl-Sn-glycero-3-phosphoethanolamine-N-[methoxy (polyethylene glycol)-2000]) or PEG2K-DMG (PEG-conjugated 1,2-Dimyristoyl-sn-glycerol, methoxypolyethylene Glycol-2000). A mixture of DSPC, DlinDMA, PEG-DMG and cholesterol is particularly effective. Other useful LNPs are described in the following references: WO2012/006376; WO2012/030901; WO2012/031046; WO2012/031043; WO2012/006378; WO2011/076807; WO2013/033563; WO2013/006825; WO2014/136086; WO2015/095340; WO2015/095346; WO2016/037053. In some embodiments, the LNPs are RV01 liposomes, see the following references: WO2012/006376 and Geall et al. (2012) PNAS USA. September 4; 109(36): 14604-9. In an embodiment, the LNPs are RV01 liposomes wherein the cationic lipid is DLin-DMA.

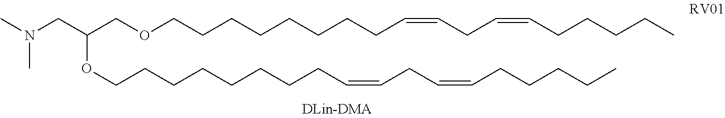 [SMILES: CCCCC/C=C\C/C=C\CCCCCCCCOCC(CN(C)C)OCCCCCCCC/C=C\C/C=C\CCCCC]

In some embodiments, the cationic lipids herein comprise the structure of Formula I:

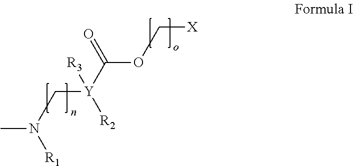

wherein n=an integer from 1 to 3 and
(i) R₁ is CH₃, R₂ and R₃ are both H, and Y is C; or
(ii) R₁ and R₂ are collectively CH₂—CH₂ and together with the nitrogen form a five-, six-, or seven-membered heterocycloalkyl, R₃ is CH₃, and Y is C; or
(iii) R₁ is CH₃, R₂ and R₃ are both absent, and Y is O;
wherein o is 0 or 1;
wherein X is:
(i)

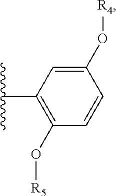

wherein R₄ and R₅ are independently a C₁₀₋₂₀ hydrocarbon chain having one or two cis alkene groups at either or both of the omega 6 and 9 positions; or
(ii) —CH(—R₆)—R₇, wherein
   (1) R₆ is —(CH₂)ₚ—O—C(O)—R₈ or —Cₚ—R₈;
   (2) R₇ is —(CH₂)ₚ—O—C(O)—R₈′ or —Cₚ—R₈′,
   (3) p and p′ are independently 0, 1, 2, 3 or 4; and
   (4) R₈ and R₈′ are independently a
      (A) —C₈₋₂₀ hydrocarbon chain having one or two cis alkene groups at either or both of the omega 6 and 9 positions;
      (B) —C₁₋₃—C(—O—C₆₋₁₂)—O—C₆₋₁₂ saturated or unsaturated hydrocarbon chain;
      (C) —C₆₋₁₆ saturated hydrocarbon chain;
      (D) —C(—C₆₋₁₆)—C₆₋₁₆ saturated or unsaturated hydrocarbon chain;
      (E) —C[—C—O—C(O)—C₄₋₁₂]—C—O—C(O)—C₄₋₁₂ saturated or unsaturated hydrocarbon chain; and
      (F) —C₆₋₁₆ saturated or unsaturated hydrocarbon chain.

In an embodiment, R₁ is CH₃, R₂ and R₃ are both H, and Y is C. In some embodiments, R₁ and R₂ are collectively CH₂—CH₂ and together with the nitrogen form a five-, six-, or seven-membered heterocycloalkyl, R₃ is CH₃, and Y is C. In some embodiments, R₁ is CH₃, R₂ and R₃ are both absent, and Y is O.

In an embodiment, X is

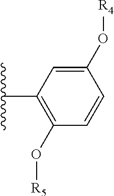

wherein R₄ and R₅ are independently a C₁₀₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is a —C₆₋₁₆ saturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ —Cₚ—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is Cₚ—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is a —C₆₋₁₆ saturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —(CH₂)ₚ—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —Cₚ—R₈, R₇ —Cₚ—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —C_{p′}—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ —Cₚ—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ —Cₚ—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ —Cₚ—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ —Cₚ—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ —Cₚ—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In an embodiment, an exemplary cationic lipid is RV28 having the following structure:

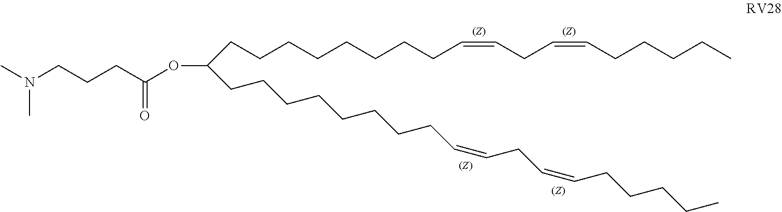 [SMILES: CCCCC/C=C\C/C=C\CCCCCCCCC(CCCCCCCC/C=C\C/C=C\CCCCC)OC(=O)CCCN(C)C]

In an embodiment, an exemplary cationic lipid is RV31 having the following structure:

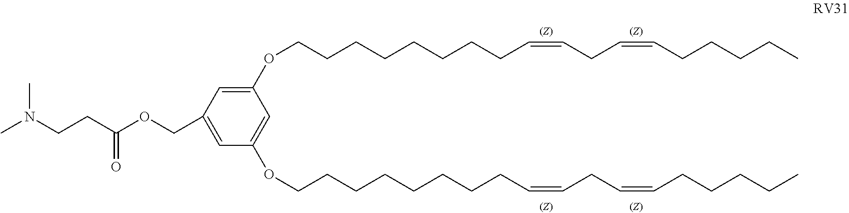 [SMILES: CCCCC/C=C\C/C=C\CCCCCCCCOc1cc(COC(=O)CCN(C)C)cc(OCCCCCCCC/C=C\C/C=C\CCCCC)c1]

In an embodiment, an exemplary cationic lipid is RV33 having the following structure:

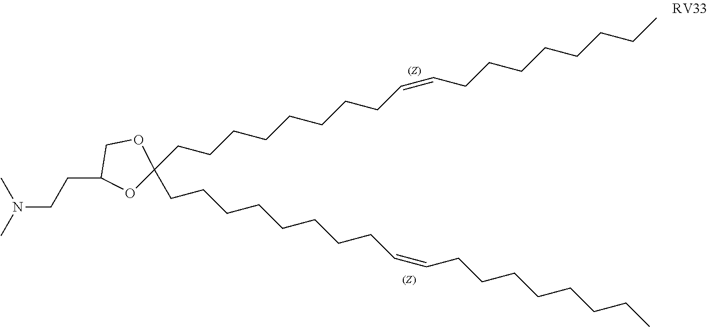 [SMILES: CCCCCCCC/C=C\CCCCCCCCC1(CCCCCCCC/C=C\CCCCCCCC)OCC(CCN(C)C)O1]

In an embodiment, an exemplary cationic lipid is RV37 having the following structure:

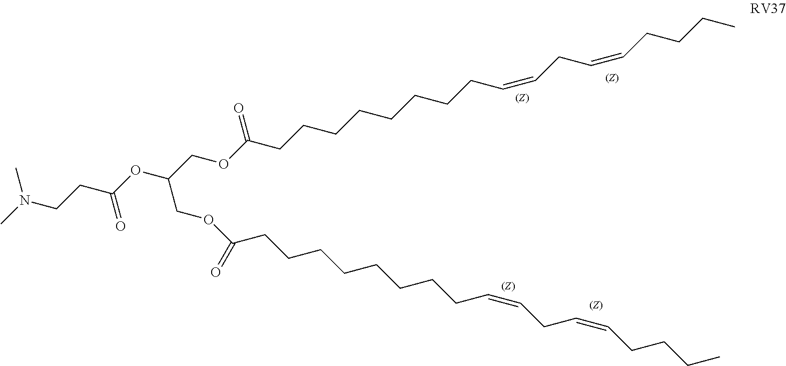 [SMILES: CCCC/C=C\C/C=C\CCCCCCCCC(=O)OCC(COC(=O)CCCCCCCC/C=C\C/C=C\CCCC)OC(=O)CCN(C)C]

In an embodiment, the LNP comprises the cationic lipid RV39, i.e., 2,5-bis((9Z,12Z)-octadeca-9,12-dien-1-yloxy)benzyl 4-(dimethylamino)butanoate):

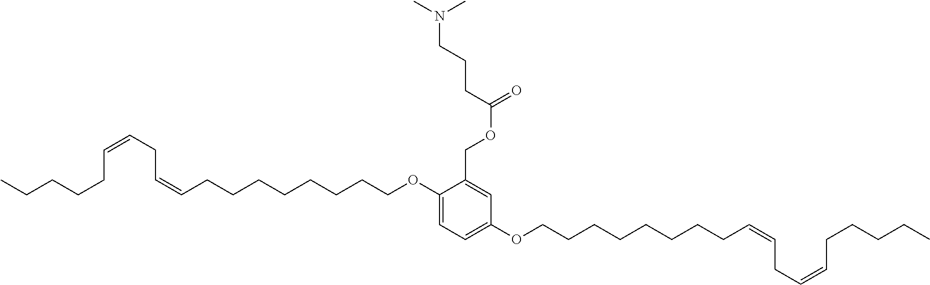 [SMILES: CCCCC/C=C\C/C=C\CCCCCCCCOc1ccc(OCCCCCCCC/C=C\C/C=C\CCCCC)c(COC(=O)CCCN(C)C)c1]

In an embodiment, an exemplary cationic lipid is RV42 having the following structure:

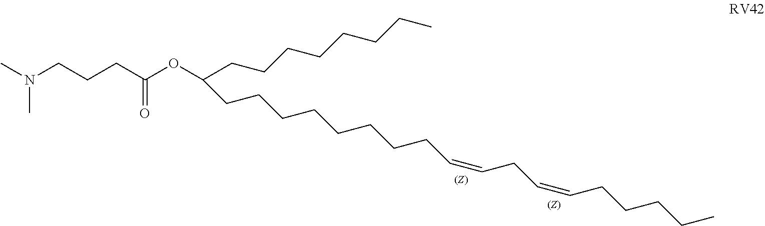 [SMILES: CCCCC/C=C\C/C=C\CCCCCCCCC(CCCCCCCC)OC(=O)CCCN(C)C]

In an embodiment, an exemplary cationic lipid is RV44 having the following structure:

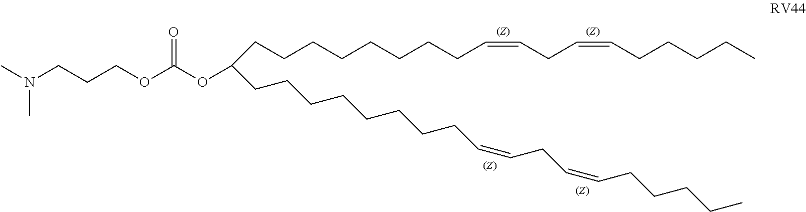 [SMILES: CCCCC/C=C\C/C=C\CCCCCCCCC(CCCCCCCC/C=C\C/C=C\CCCCC)OC(=O)OCCCN(C)C]

In an embodiment, an exemplary cationic lipid is RV73 having the following structure:

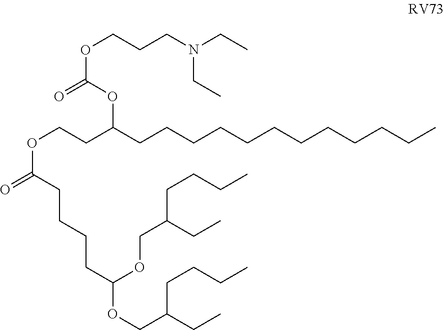 [SMILES: CCCCCCCCCCCCC(CCOC(=O)CCCCC(OCC(CC)CCCC)OCC(CC)CCCC)OC(=O)OCCCN(CC)CC]

In an embodiment, an exemplary cationic lipid is RV75 having the following structure:

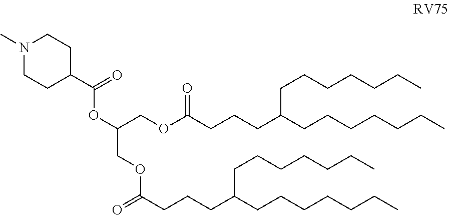 [SMILES: CCCCCCCC(CCCCCCC)CCCC(=O)OCC(COC(=O)CCCC(CCCCCCC)CCCCCCC)OC(=O)C1CCN(C)CC1]

In an embodiment, an exemplary cationic lipid is RV81 having the following structure:

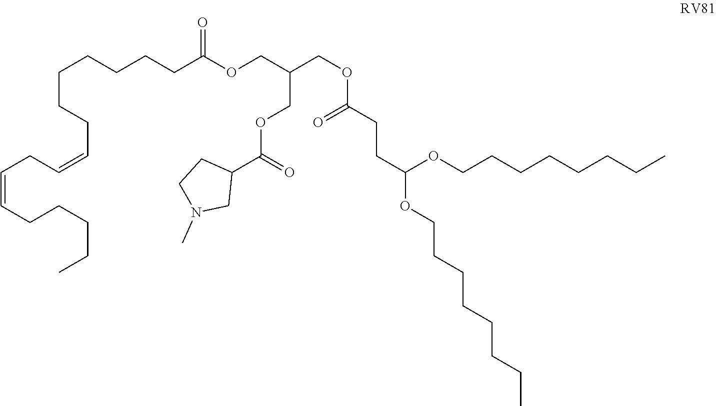 [SMILES: CCCCC/C=C\C/C=C\CCCCCCCC(=O)OCC(COC(=O)CCC(OCCCCCCCC)OCCCCCCCC)COC(=O)C1CCN(C)C1]

In an embodiment, an exemplary cationic lipid is RV84 having the following structure:

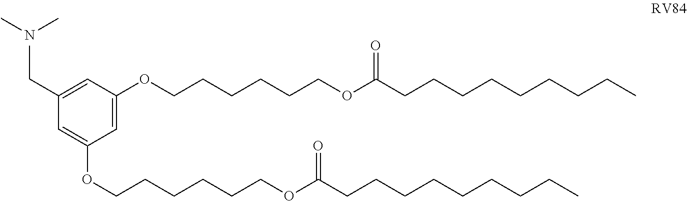

In an embodiment, an exemplary cationic lipid is RV85 having the following structure:

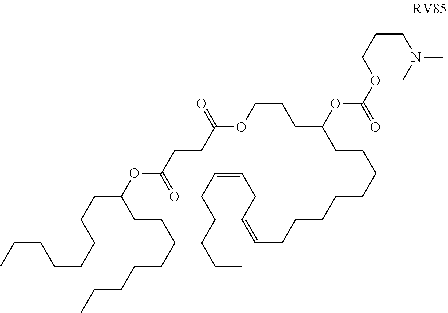 [SMILES: CCCCC/C=C\C/C=C\CCCCCCCCC(CCCOC(=O)CCC(=O)OC(CCCCCCCC)CCCCCCCC)OC(=O)OCCCN(C)C]

In an embodiment, an exemplary cationic lipid is RV86 having the following structure:

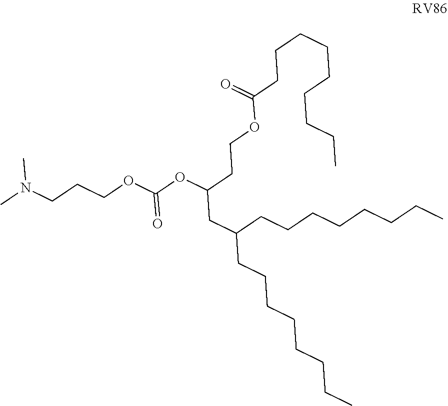 [SMILES: CCCCCCCCCC(=O)OCCC(CC(CCCCCCCC)CCCCCCCC)OC(=O)OCCCN(C)C]

In an embodiment, an exemplary cationic lipid is RV88 having the following structure:

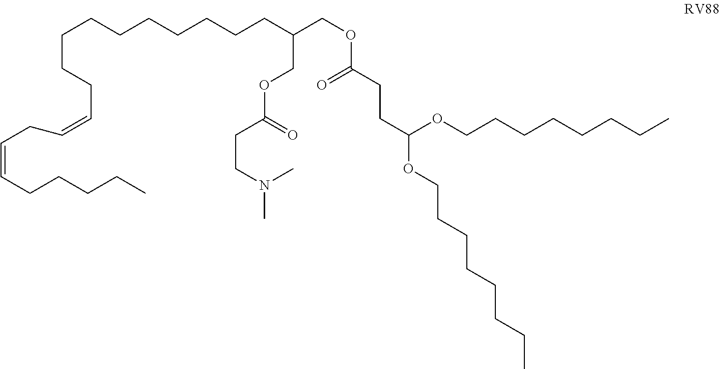 [SMILES: CCCCC/C=C\C/C=C\CCCCCCCCCCC(COC(=O)CCC(OCCCCCCCC)OCCCCCCCC)COC(=O)CCN(C)C]

In an embodiment, an exemplary cationic lipid is RV91 having the following structure:

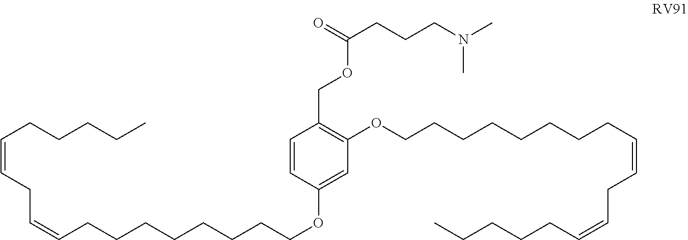 [SMILES: CCCCC/C=C\C/C=C\CCCCCCCCOc1ccc(COC(=O)CCCN(C)C)c(OCCCCCCCC/C=C\C/C=C\CCCCC)c1]

In an embodiment, an exemplary cationic lipid is RV92 having the following structure:

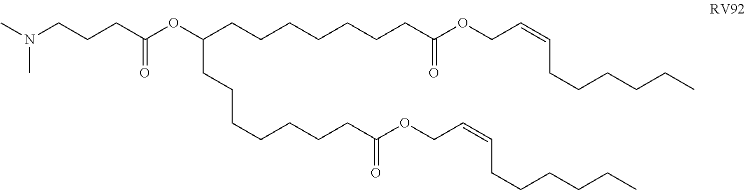 [SMILES: CCCCCC/C=C\COC(=O)CCCCCCCC(CCCCCCCC(=O)OC/C=C\CCCCCC)OC(=O)CCCN(C)C]

In an embodiment, an exemplary cationic lipid is RV93 having the following structure:

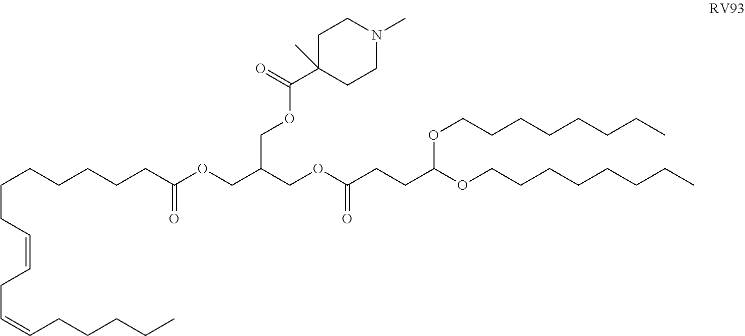 [SMILES: CCCCC/C=C\C/C=C\CCCCCCCC(=O)OCC(COC(=O)CCC(OCCCCCCCC)OCCCCCCCC)COC(=O)C1(C)CCN(C)CC1]

In an embodiment, an exemplary cationic lipid is 2-(5-((4-((1,4-dimethylpiperidine-4-carbonyl)oxy)hexadecyl)oxy)-5-oxopentyl)propane-1,3-diyl dioctanoate (RV94), having the following structure:

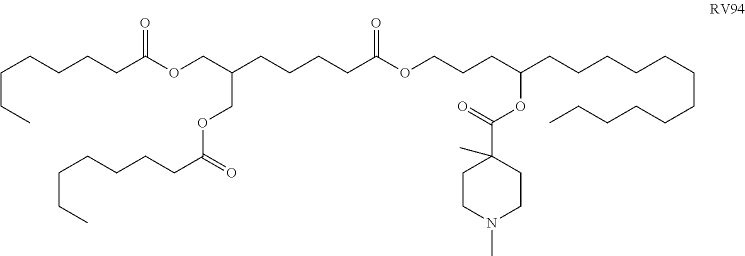 [SMILES: CCCCCCCCCCCCC(CCCOC(=O)CCCCC(COC(=O)CCCCCCC)COC(=O)CCCCCCC)OC(=O)C1(C)CCN(C)CC1]

In an embodiment, an exemplary cationic lipid is RV95 having the following structure:

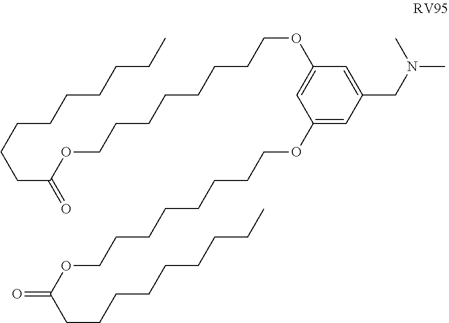 [SMILES: CCCCCCCCCC(=O)OCCCCCCCCOc1cc(CN(C)C)cc(OCCCCCCCCOC(=O)CCCCCCCCC)c1]

In an embodiment, an exemplary cationic lipid is RV96 having the following structure:

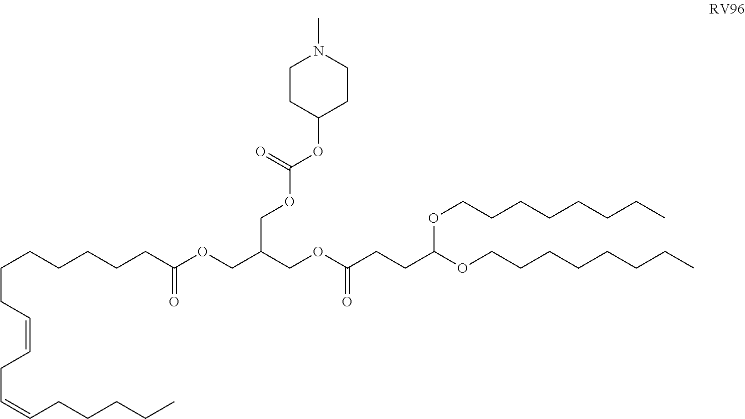 [SMILES: CCCCC/C=C\C/C=C\CCCCCCCC(=O)OCC(COC(=O)CCC(OCCCCCCCC)OCCCCCCCC)COC(=O)OC1CCN(C)CC1]

In an embodiment, an exemplary cationic lipid is RV97 having the following structure:

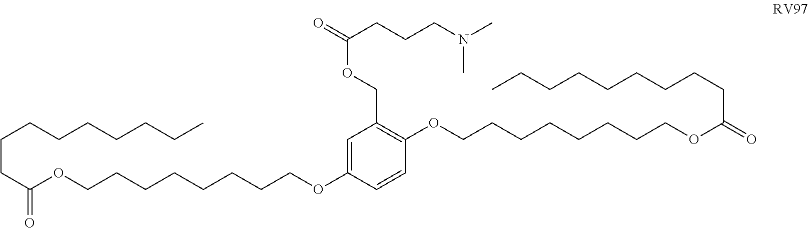 [SMILES: CCCCCCCCCC(=O)OCCCCCCCCOc1ccc(OCCCCCCCCOC(=O)CCCCCCCCC)c(COC(=O)CCCN(C)C)c1]

In an embodiment, an exemplary cationic lipid is RV99 having the following structure:

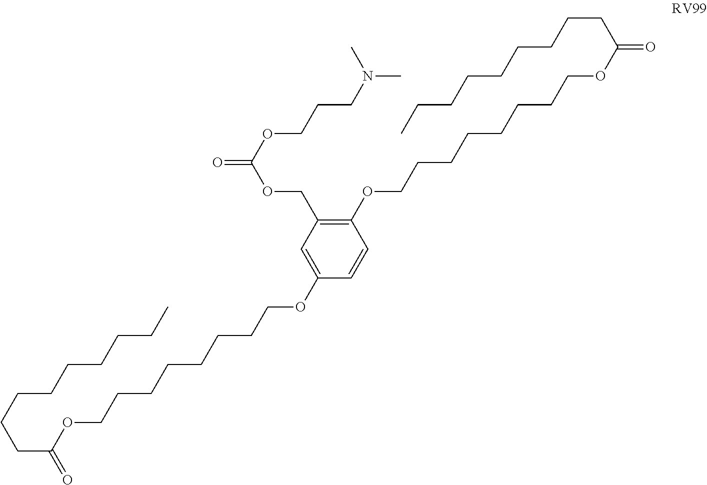 [SMILES: CCCCCCCCCC(=O)OCCCCCCCCOc1ccc(OCCCCCCCCOC(=O)CCCCCCCCC)c(COC(=O)OCCCN(C)C)c1]

In an embodiment, an exemplary cationic lipid is RV101 having the following structure:

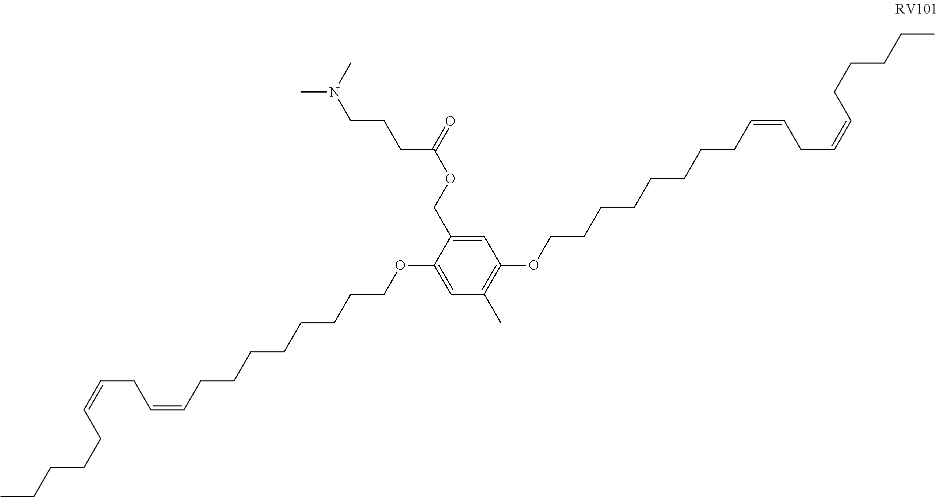 [SMILES: CCCCC/C=C\C/C=C\CCCCCCCCOc1cc(COC(=O)CCCN(C)C)c(OCCCCCCCC/C=C\C/C=C\CCCCC)cc1C]

In an embodiment, the cationic lipid is selected from the group consisting of: RV28, RV31, RV33, RV37, RV39, RV42, RV44, RV73, RV75, RV81, RV84, RV85, RV86, RV88, RV91, RV92, RV93, RV94, RV95, RV96, RV97, RV99, and RV101. In an embodiment, the cationic lipid is selected from the group consisting of: RV39, RV88, and RV94.

Compositions and methods for the synthesis of compounds having Formula I and RV28, RV31, RV33, RV37, RV39, RV42, RV44, RV73, RV75, RV81, RV84, RV85, RV86, RV88, RV91, RV92, RV93, RV94, RV95, RV96, RV97, RV99, and RV101 can be found in PCT/US2014/070882 (publication number WO/2015/095340) and PCT/US2014/070891 (publication number WO/2015/095346), filed 17 Dec. 2014; as well as PCT/US2015/048535 (publication number WO/2016/037053), filed 4 Sep. 2015.

In some embodiments, the methods of manufacturing a non-viral delivery system comprising a liposome encapsulating an RNA comprise the following steps: (a) mixing (i) a first solution comprising a solvent, an ionizable cationic lipid, a zwitterionic lipid, a sterol, and a PEGylated lipid selected; and (ii) a second solution comprising water and the RNA; and (b) removing the solvent. The mixing may be carried out in a T-junction device, a microfluidic device, or the like, as described in WO2012031046 and/or PCT/IB2018/053850.

The immunogenic composition according to the invention may be a pharmaceutical composition e.g. a vaccine composition. Accordingly, the composition may also comprise a pharmaceutically acceptable carrier.

A “pharmaceutically acceptable carrier” includes any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The compositions may also contain a pharmaceutically acceptable diluent, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier.

Pharmaceutical compositions may include the immunogenic compositions, nucleic acid sequences, and/or polypeptide sequences described elsewhere herein in plain water (e.g. “w.f.i.”) or in a buffer e.g. a phosphate buffer, a Tris buffer, a borate buffer, a succinate buffer, a histidine buffer, or a citrate buffer. Buffer salts will typically be included in the 5-20 mM range. Pharmaceutical compositions may have a pH between 5.0 and 9.5 e.g. between 6.0 and 8.0. Compositions may include sodium salts (e.g. sodium chloride) to give tonicity. A concentration of 10±2 mg/mL NaCl is typical, e.g. about 9 mg/mL. Compositions may include metal ion chelators. These can prolong RNA stability by removing ions which can accelerate phosphodiester hydrolysis. Thus a composition may include one or more of EDTA, EGTA, BAPTA, pentetic acid, etc. Such chelators are typically present at between 10-500 μM e.g. 0.1 mM. A citrate salt, such as sodium citrate, can also act as a chelator, while advantageously also providing buffering activity. Pharmaceutical compositions may have an osmolality of between 200 mOsm/kg and 400 mOsm/kg, e.g. between 240-360 mOsm/kg, or between 290-310 mOsm/kg. Pharmaceutical compositions may include one or more preservatives, such as thiomersal or 2-phenoxyethanol. Mercury-free compositions are preferred, and preservative-free vaccines can be prepared. Pharmaceutical compositions may be aseptic or sterile. Pharmaceutical compositions may be non-pyrogenic e.g. containing <1 EU (endotoxin unit, a standard measure) per dose, and preferably <0.1 EU per dose. Pharmaceutical compositions may be gluten free. Pharmaceutical compositions may be prepared in unit dose form. In some embodiments a unit dose may have a volume of between 0.1-1.0 mL e.g. about 0.5 mL.

In some embodiments, the compositions disclosed herein are immunogenic composition that, when administered to a subject, induce a humoral and/or cellular antigen-specific immune response (i.e. an immune response which specifically recognizes a naturally occurring influenza virus antigen polypeptide). For example, an immunogenic composition may induce a memory T and/or B cell population relative to an untreated subject following influenza virus infection. In some embodiments, the subject is a vertebrate, such as a mammal e.g. a human or a veterinary mammal.

The immunogenic compositions of the invention can be formulated as vaccine compositions. The vaccine will comprise an immunologically effective amount of antigen. By “an immunologically effective amount” is intended that the administration of that amount to a subject, either in a single dose or as part of a series, is effective for inducing a measurable immune response against influenza virus in the subject. This amount varies depending upon the health and physical condition of the individual to be treated, age, the taxonomic group of individual to be treated (e.g. human, non-human primate, etc.), the capacity of the individual's immune system to synthesize antibodies, the degree of protection desired, the formulation of the composition or vaccine, the treating doctor's assessment of the medical situation, the severity of the disease, the potency of the compound administered, the mode of administration, and other relevant factors. It is expected that the amount will fall in a relatively broad range that can be determined through routine trials. Vaccines as disclosed herein may either be prophylactic (i.e. to prevent infection) or therapeutic (i.e. to treat infection), but will typically be prophylactic. In some embodiments, the vaccine compositions disclosed herein may induce an effective immune response against an influenza virus infection, i.e., a response sufficient for treatment or prevention of a influenza virus infection.

In some embodiments, the immunogenic composition of the invention further comprises an additional antigen. In some embodiments, the immunogenic composition is administered to a subject in combination with a further composition which comprises an additional antigen.

In a specific embodiment is provided a pharmaceutical composition (such as a vaccine composition) which comprises or consists of (i) from 3 to 10 self-replicating RNA molecules wherein each self-replicating RNA molecule encodes a polypeptide comprising an antigen from influenza virus, wherein each antigen is from a different strain of influenza virus to the other antigens and wherein the self-replicating RNA molecules are formulated in lipid nanoparticles (LNP) (iii) a pharmaceutical carrier, diluent and/or buffer and optionally (iii) an adjuvant.

For example, a vaccine composition is provided which comprises or consists of (i) from 3 to 10 self-replicating RNA molecules formulated in lipid nanoparticles (LNP) and (ii) a pharmaceutical carrier, diluent and/or buffer, wherein:
each self-replicating RNA molecule encodes a polypeptide comprising an antigen from influenza virus,
each antigen is from a different strain of influenza virus to the other antigens and
the LNP comprises a neutral lipid, a cationic lipid, cholesterol and polyethylene glycol (PEG) which form nanoparticles that encompass the self-replicating RNA. In certain embodiments, the neutral lipid is DSPC and the cationic lipid is DLinDMA.

An immunogenic composition of the present invention may also comprise, or be administered in conjunction with, one or more adjuvants (e.g. vaccine adjuvants). By adjuvant is intended that it is capable of increasing an immune response against an antigen compared to administration of said antigen alone. In some aspects, adjuvant compositions as disclosed herein further comprise one or more immunostimulants, for example, a saponin such as QS21.

Adjuvants which may be used in compositions of the invention include, but are not limited to: (A) Mineral-containing compositions, for example aluminum and calcium salts, such as aluminum phosphates. (B) Oil emulsions, for example squalene-in-water emulsions, such as MF59 or AS03. Complete Freund's adjuvant (CFA) and incomplete Freund's adjuvant (IF A) may also be used. (C) Saponin formulations. (D) Virosomes and virus-like particles (VLPs). (E) Bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof. (F) Human immunomodulators, for example cytokines, such as interleukins, interferons, macrophage colony stimulating factor, and tumor necrosis factor. (G) Bioadhesives and mucoadhesives, such as esterified hyaluronic acid microspheres, cross-linked derivatives of poly(acrylic acid), polyvinyl alcohol, polyvinyl pyrollidone, polysaccharides and carboxymethylcellulose. (H) Microparticles, for example particles of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(a-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB). (I) Liposomes. (J) Polyoxyethylene ether and polyoxyethylene ester formulations. (K) Polyphosphazene (PCPP). (L) Muramyl peptides. (M) Imidazoquinolone compounds, for example Imiquamod and its homologues.

Combinations of one or more of the adjuvants identified above may also be used with the invention.

In a specific embodiment is provided a vaccine composition according to the invention

### Administration and Uses

### Methods of Use/Uses

In some embodiments are provided methods for inducing an immune response against influenza virus infection in a subject in need thereof comprising a step of administering an immunologically effective amount of the immunogenic or pharmaceutical compositions as disclosed herein.

In some embodiments are provided the use of the compositions disclosed herein for inducing an immune response to an influenza virus antigen in a subject in need thereof. In some embodiments are provided the use of the compositions disclosed herein for inducing an immune response against an influenza virus infection in a subject. In some embodiments are provided use of the compositions as disclosed herein in the manufacture of a medicament that induces an immune response to a influenza virus infection in a subject.

By “subject” is intended a vertebrate, such as a mammal e.g. a human or a veterinary mammal. In some embodiments the subject is human. By “immune response” is intended a humoral and/or cellular antigen-specific immunological response (i.e. an immune response which specifically recognizes an antigen polypeptide) that can be demonstrated to neutralize influenza virus in vitro or control/reduce/eliminate infection virus infection in vivo. In some embodiments, the immune response is characterized by immunological memory against the influenza virus and/or an effective influenza virus-responsive memory T cell population.

In some embodiments, the compositions disclosed herein are for use in therapy or medicine. In a preferred embodiment, the therapy is a vaccine therapy. Preferably the therapy is a vaccine to prevent influenza virus infection. In some embodiments a composition disclosed herein is for use in preventing or treating influenza or for use in preventing or treating influenza virus infection in a subject in need thereof. In some embodiments, a composition disclosed herein is for use in inducing an immune response against a influenza virus infection in a subject in need thereof.

A composition described herein may be for use in preventing influenza virus infection by multiple different strains of influenza virus, or for inducing an immune response to an infection by any one of multiple different strains of influenza virus. For example, a composition may be for use in preventing or shortening influenza virus infection against two or more H1 and/or two or more H3 type strains of influenza virus. The composition may be for preventing or shortening influenza virus infection against both seasonal and pandemic strains of influenza virus. In some embodiments, the composition described herein may be for use in preventing influenza virus infection against homologous and/or heterologous strains of influenza virus. In one embodiment, the composition may be for use in preventing influenza virus infection against intrasubtypic and/or heterosubtypic strains of influenza virus.

Hence, in certain embodiments is provided an immunogenic or pharmaceutical composition as disclosed herein for use in preventing influenza virus infection against intrasubtypic strains of influenza virus.

In a specific embodiment, is provided a vaccine composition for use in preventing influenza virus infection against homologous and/or heterologous strains of influenza virus wherein the vaccine composition comprises (i) a first self-replicating RNA molecule encoding a polypeptide comprising a first antigen and (ii) a second self-replicating RNA molecule encoding a polypeptide comprising a second antigen, wherein:
the first and second antigens are both from influenza virus but the first antigen is from a different strain of influenza virus to the second antigen,
the first and second antigens are hemagglutinin or an immunogenic fragment or variant thereof and
the self-replicating RNA molecules are formulated in lipid nanoparticles (LNP) or a cationic nanoemulsion (CNE).

In a specific embodiment is provided a vaccine composition for use in preventing influenza virus infection against homologous and/or heterologous strains of influenza virus wherein the vaccine composition comprises (i) a first self-replicating RNA molecule encoding a polypeptide comprising a first antigen and (ii) a second self-replicating RNA molecule encoding a polypeptide comprising a second antigen, wherein:
the first and second antigens are both from influenza virus but the first antigen is from a different strain of influenza virus to the second antigen,
the first and second antigens are hemagglutinin or an immunogenic fragment or variant thereof,
the self-replicating RNA molecules are formulated in lipid nanoparticles (LNP) or a cationic nanoemulsion (CNE) and
as well as encoding a polypeptide comprising an antigen from influenza virus, each self-replicating RNA molecule encodes a RNA-dependent RNA polymerase which can transcribe RNA from the self-replicating RNA molecule.

In a further specific embodiment is provided a vaccine composition for use in preventing influenza virus infection against homologous and/or heterologous intrasubtypic strains of influenza virus wherein the vaccine composition comprises (i) a first self-replicating RNA molecule encoding a polypeptide comprising a first antigen and (ii) a second self-replicating RNA molecule encoding a polypeptide comprising a second antigen, wherein:
the first and second antigens are both from influenza virus but the first antigen is from a different strain of influenza virus to the second antigen,
the first and second antigens are hemagglutinin or an immunogenic fragment or variant thereof and
as well as encoding a polypeptide comprising an antigen from influenza virus, each self-replicating RNA molecule encodes a RNA-dependent RNA polymerase which can transcribe RNA from the self-replicating RNA molecule.

In a further specific embodiment is provided a vaccine composition for use in preventing influenza virus infection against homologous and/or heterologous intrasubtypic strains of influenza virus wherein the vaccine composition comprises (i) a first self-replicating RNA molecule encoding a polypeptide comprising a first antigen (ii) a second self-replicating RNA molecule encoding a polypeptide comprising a second antigen, and (iii) a third self-replicating RNA molecule encoding a polypeptide comprising a third antigen, wherein:
the first, second and third antigens are both from influenza virus but the first, second and third antigens are all from different strains of influenza virus,
the first, second and third antigens are hemagglutinin or an immunogenic fragment or variant thereof and
the self-replicating RNA molecules are formulated in lipid nanoparticles (LNP) or a cationic nanoemulsion (CNE).

In a further specific embodiment is provided a vaccine composition for use in preventing influenza virus infection against homologous and/or heterologous intrasubtypic strains of influenza virus wherein the vaccine composition comprises (i) a first self-replicating RNA molecule encoding a polypeptide comprising a first antigen (ii) a second self-replicating RNA molecule encoding a polypeptide comprising a second antigen, and (iii) a third self-replicating RNA molecule encoding a polypeptide comprising a third antigen, wherein:
the first, second and third antigens are both from influenza virus but the first, second and third antigens are all from different strains of influenza virus,
the first, second and third antigens are hemagglutinin or an immunogenic fragment or variant thereof, and
as well as encoding a polypeptide comprising an antigen from influenza virus, each self-replicating RNA molecule encodes a RNA-dependent RNA polymerase which can transcribe RNA from the self-replicating RNA molecule.

In a further specific embodiment is provided a vaccine composition for use in preventing influenza virus infection against homologous and/or heterologous intrasubtypic strains of influenza virus wherein the vaccine composition comprises (i) a first self-replicating RNA molecule encoding a polypeptide comprising a first antigen (ii) a second self-replicating RNA molecule encoding a polypeptide comprising a second antigen, and (iii) a third self-replicating RNA molecule encoding a polypeptide comprising a third antigen, wherein:
the first, second and third antigens are both from influenza virus but the first, second and third antigens are all from different strains of influenza virus,
the first, second and third antigens are hemagglutinin or an immunogenic fragment or variant thereof,
the self-replicating RNA molecules are formulated in lipid nanoparticles (LNP) or a cationic nanoemulsion (CNE) and
as well as encoding a polypeptide comprising an antigen from influenza virus, each self-replicating RNA molecule encodes a RNA-dependent RNA polymerase which can transcribe RNA from the self-replicating RNA molecule.

In some embodiments, methods are provided for preventing or shortening influenza virus infection and/or reducing or preventing the clinical symptoms upon influenza virus infection in a subject in need thereof, which comprises administering to said subject an immunologically effective amount of an immunogenic composition as provided herein.

In some embodiments, methods are provided for preventing or shortening influenza virus infection against multiple different strains of influenza virus. For example, the methods may be for preventing or shortening influenza virus infection against two or more H1 and/or two or more H3 type strains of influenza virus and optionally against two or more strains of influenza B virus. The methods may be for preventing or shortening influenza virus infection against both seasonal and pandemic strains of influenza virus. In some embodiments, the methods described herein may be for use in preventing influenza virus infection against homologous and/or heterologous strains of influenza virus.

In some embodiments is provided use of a composition disclosed herein in the manufacture of an immunogenic composition for preventing or shortening influenza virus infection in a subject and/or reducing or prevent the clinical symptoms upon influenza virus infection in a subject.

In some embodiments, the subject is a human subject. In specific embodiments, the human subject has been exposed, or is at risk of being exposed, to an influenza virus infection.

In some embodiments, multiple compositions comprising one or more self-replicating RNA molecules that encode a polypeptide comprising an antigen from influenza virus may be used. Hence, there is provided is a method of prevention and/or treatment against influenza disease, comprising (i) the administration of a first immunogenic composition comprising a first self-replicating RNA molecule and pharmaceutically acceptable carrier and (ii) simultaneous or sequential administration of a second immunogenic composition comprising a second self-replicating RNA molecule and pharmaceutically acceptable carrier, wherein the first and second self-replicating RNA molecules each encode a polypeptide encoding an antigen from influenza virus but the first self-replicating RNA molecule encodes an antigen from a different strain of influenza to that encoded by the second self-replicating RNA molecule.

The compositions may be administered sequentially, for example at substantially the same time such as at an interval of less than 10 hours, from 1 second to 10 hours or from 1 second to 1 hour, or at larger intervals of from 10 hours to 6 months, from 10 hours to 1 month, from 10 hours to 2 weeks, from 10 hours to 1 week or from 10 hours to 1 day. Preferably, the sequential administration is at an interval of from 1 second to 10 hours. The first or second immunogenic composition may comprise one or more additional (e.g. a 3rd, 4th, 5th, 6th, 7th, 8th, 9th and/or 10th) self-replicating RNA molecules each encoding a polypeptide comprising an antigen from influenza virus, but wherein the antigen in each self-replicating RNA molecule is from a different strain of influenza virus to the other antigens from influenza virus.

Hence, in a specific embodiment is provided is a method of prevention and/or treatment against influenza disease, comprising (i) the administration of a first immunogenic composition comprising a first self-replicating RNA molecule and pharmaceutically acceptable carrier and (ii) simultaneous or sequential administration of a second immunogenic composition comprising a second self-replicating RNA molecule and pharmaceutically acceptable carrier, wherein:
the first and second self-replicating RNA molecules each encode a polypeptide encoding an antigen from influenza virus but the first self-replicating RNA molecule encodes an antigen from a different strain of influenza to that encoded by the second self-replicating RNA molecule
the antigen from influenza virus is hemagglutinin or an immunogenic fragment or variant thereof and
the second immunogenic composition is administered from 1 day to 6 months after the first immunogenic composition.

Also provided is a first immunogenic composition comprising a first self-replicating RNA molecule and a pharmaceutically acceptable carrier for use in a method of preventing influenza disease, said method comprising administration to a subject in need the first immunogenic composition followed by administration of a second immunogenic composition comprising a self-replicating RNA molecule and a pharmaceutically acceptable carrier, wherein the first and second self-replicating RNA molecules each encode a polypeptide comprising an antigen, wherein the antigen is from influenza virus but the antigen encoded by the first self-replicating RNA molecule is from a different strain of influenza virus to that encoded by the second self-replicating RNA molecule.

The compositions may be administered at an interval of less than 10 hours, from 1 second to 10 hours or from 1 second to 1 hour, or at larger intervals of from 10 hours to 6 months, from 10 hours to 1 month, from 10 hours to 2 weeks, from 10 hours to 1 week or from 10 hours to 1 day. The first/second immunogenic composition may comprise one or more additional (e.g. a 3rd, 4th, 5th, 6th, 7th, 8th, 9th and/or 10th) self-replicating RNA molecules each encoding a polypeptide comprising an antigen from influenza virus, but wherein the antigen in each self-replicating RNA molecule is from a different strain of influenza virus to the other antigens from influenza virus.

In a specific embodiment is provided a a first immunogenic composition comprising a first self-replicating RNA molecule and a pharmaceutically acceptable carrier for use in a method of preventing influenza disease, said method comprising administration to a subject in need the first immunogenic composition followed by administration of a second immunogenic composition comprising a self-replicating RNA molecule and a pharmaceutically acceptable carrier, wherein:
the first and second self-replicating RNA molecules each encode a polypeptide comprising an antigen, wherein the antigen is from influenza virus but the antigen encoded by the first self-replicating RNA molecule is from a different strain of influenza virus to that encoded by the second self-replicating RNA molecule and
the antigen from influenza virus is hemagglutinin or an immunogenic fragment or variant thereof and
the second immunogenic composition is administered from 1 day to 6 months after the first immunogenic composition

### Routes of Administration/Dosages

Compositions disclosed herein will generally be administered directly to a subject. Direct delivery may be accomplished by parenteral injection (e.g. subcutaneously, intraperitoneally, intravenously, intramuscularly, intradermally, or to the interstitial space of a tissue). Alternative delivery routes include rectal, oral (e.g. tablet, spray), buccal, sublingual, vaginal, topical, transdermal or transcutaneous, intranasal, ocular, aural, pulmonary or other mucosal administration. Intradermal and intramuscular administration are two preferred routes. Injection may be via a needle (e.g. a hypodermic needle), but needle-free injection may alternatively be used. The dose volume may be from 0.25 ml to 1 ml, in particular 0.5 ml or 0.7 ml. Slight adaptation of the dose volume will be made routinely depending on the RNA concentration in the original bulk sample and depending also on the delivery route, with smaller doses being given by the intranasal or interdermal route. A typical human intramuscular dose volume is 0.5 ml.

A dose of a self-replicating RNA vaccine may have about 50 μg to about 100 μg nucleic acid. In one embodiment, a vaccine dose contains 50, 55, 60, 65, 70, 75, 80, 85, 90, 95 or 100 μg self-replicating RNA. In other embodiments, a dose of a composition according to the invention may have <10 μg self-replicating RNA; e.g. from 1-10 μg, such as about 1 μg, 2.5 μg, 5 μg, 7.5 μg or 10 μg, but expression can be seen at much lower levels; e.g. using <1 μg/dose, <100 ng/dose, <10 ng/dose, <1 ng/dose, etc.

In preferred embodiments, a composition disclosed herein is administered to a subject at an effective dose, meaning a dose sufficient to achieve a desired immune response, such as induction of neutralizing antibodies to influenza virus and/or protection against influenza virus infection.

In some embodiments, a composition described herein (such as a vaccine composition) has an effective dose that is less than or equal to 50%, 40%, 30%, 20% or 10% of the effective dose of a DNA vaccine or vaccine composition encoding the same antigen. In some embodiments, a vaccine described herein has an effective dose that is one third or less of the effective dose of a DNA vaccine or vaccine composition encoding the same antigen.

### Processes of Manufacture/Formulation

Processes for the manufacture of self-replicating RNA are provided herein. In some embodiments, the process of manufacturing a self-replicating RNA comprises a step of in vitro transcription (IVT) as described elsewhere herein. In some embodiments, the process of manufacturing a self-replicating RNA comprises a step of IVT to produce a RNA, and further comprises a step of combining the RNA with a non-viral delivery system as described elsewhere herein. In some embodiments, the process of manufacturing a self-replicating RNA comprises a step of IVT to produce a RNA, and further comprises a step of combining the RNA with a CNE or LNP delivery system as described elsewhere herein.

### Sequence Identity

Identity or homology with respect to an amino acid sequence is defined herein as the percentage of amino acid residues in the candidate sequence that are identical with the reference amino acid sequence after aligning the sequences and introducing gaps, if necessary, to achieve the maximum percent sequence identity, and not considering any conservative substitutions as part of the sequence identity. Identity or homology with respect to a nucleic acid sequence is defined herein as the percentage of nucleotides in the candidate sequence that are identical with the reference nucleic acid sequence after aligning the sequences and introducing gaps, if necessary, to achieve the maximum percent sequence identity.

Sequence identity can be determined by standard methods that are commonly used to compare the similarity in position of the amino acids of two polypeptides. Using a computer program such as BLAST, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the shorter sequences in order to align the two sequences. The same methods used to compare polypeptides can also be used to calculate the percent identity of two polynucleotide sequences.

Where the present disclosure refers to a sequence by reference to a UniProt or Genbank accession code, the sequence referred to is the current version at the filing date of the present application.

### General

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “plurality” refers to two or more. Additionally, numerical limitations given with respect to concentrations or levels of a substance, such as solution component concentrations or ratios thereof, and reaction conditions such as temperatures, pressures and cycle times are intended to be approximate. The term “about” used herein is intended to mean the amount ±10%.

The term “comprises” means “includes.” Thus, unless the context requires otherwise, the word “comprises,” and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or groups thereof. Embodiments described as comprising certain components are intended to include embodiments consisting of the indicated components.

The abbreviation, “e.g.” is derived from the Latin exempli gratia, and is used herein to indicate a non-limiting example. Thus, the abbreviation “e.g.” is synonymous with the term “for example.”

The invention will be further described by reference to the following, non-limiting, figures and examples.

### EXAMPLES

### Example 1—Materials and Methods

### Selection of Influenza H3N2 Vaccine Strains

Potential pandemic and seasonal strains of the influenza H3N2 subtypes were selected on the basis of three major criteria 1) major glycosylation sites 2) important seasonal and pandemic vaccine strains 3) cell adapted strains only. Sequence information for full length genes encoding for influenza HA proteins was obtained from the Global Initiative on Sharing All Influenza Data Epiflu database (www.gisaid.org). All together 6 H3N2 HA gene segments from A/Bilthoven/16398/1968 (EPI362379), A/Bangkok/1/79 (EPI367158), A/Beijing/32/92 (EPI365898), A/Fujian/411/2002 (EPI362915), A/Brisbane/10/2007 (EPI362338) and A/Texas/50/2012 (EPI398417) were selected.

| Strains | EPI-Segment-ID* |
|---|---|
| A/California/07/2009 (H1N1) | EPI516528 |
| A/PR/8/34 (H1N1) | EPI131282 |
| A/turkey/Turkey/1/2005 (H5N1) | EPI102703 |
| A/Bilthoven/16398/1968 (H3N2) | EPI362379 |
| A/Bangkok/1/79 (H3N2) | EPI367158 |
| A/Beijing/32/92 (H3N2) | EPI365898 |
| A/Fujain/411/2002 (H3N2) | EPI362915 |
| A/Brisbane/10/2007 (H3N2) | EPI362338 |
| A/Texas/50/2012 (H3N2) | EPI398417 |
| A/Anhui/1/2013 (H7N9) | EPI539507 |
| A/Memphis/1/1971 (H3N2) | EPI137302 |
| A/Moscow/10/1999 (H3N2) | EPI103359 |
| A/Japan/305/1957 (H2N2) | EPI240974 |
| A/duck/Hongkong/562/1979 (H10N9) | EPI42118 |

*Source: Global Initiative on Sharing All Influenza Data Epiflu database (www.gisaid.org)

### SAM Replicons and RNA Synthesis

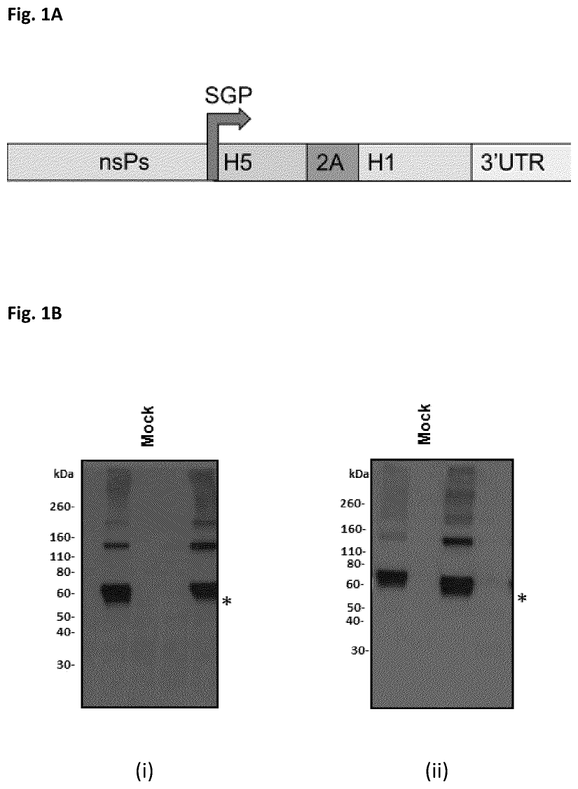

Monocistronic SAM(HA) vaccine constructs, SAM (H1-Cal), SAM (H1-PR8), SAM (H5-turkey), SAM (H3-Biltho), SAM (H3-Bang), SAM (H3-Beij), SAM (H3-Fuj), SAM (H3-Bris), SAM (H3-Tex) and SAM (H7-Shan), were generated after cloning full length HA gene segment of A/California/07/2009 (H1N1), A/PR/8/1934 (H1N1), A/turkey/Turkey/5/2005 (H5N1), A/Bilthoven/16398/1968 (H3N2), A/Bangkok/1/79 (H3N2), A/Beijing/32/92 (H3N2), A/Fujian/411/2002 (H3N2), A/Brisbane/10/2007 (H3N2), A/Texas/50/2012 (H3N2) and A/Shanghai/1/2013 (H7N9), respectively in an alphavirus based SAM vector TC83. Similarly, a bicistronic SAM construct (FIG. 1a) harboring two HA genes, H5 and H1, was produced by cloning the second HA gene downstream of the full length 2A-driven sequence using a splicing by overlap extension (SOEing) method with monomeric HA forms as primary PCR templates. The primers used for the SOEIng PCRs are shown below:

catagtctagtcgacgccaccatggagaaaatagtgcttcttcttgc
(SB63)
gtcgaagttcagggtctgcttcacgggggccacgatcttctgcttgtgcc
gggcctcccgcttggcccgaatgcaaattctgcattgtaacgatc
(SB74)
gtgaagcagaccctgaacttcgacctgctgaagctggccggcgacgtgga
gagcaaccccggccccatgaaggcaatactagtagttctgc (SB76)
ggcgtagcggcggccgcttatcaaatacatattctacactgtagagaccc
a (SB66)

Self-amplifying mRNAs were prepared as previously described (Hekele et al Emerg Microbes Infect 2013; 2 doi:ARTN e52DOI 10.1038/emi.2013.54) Briefly, full length HA genes were either chemically synthesized (GeneArt, Thermo Fischer, USA) or amplified from the reverse-transcribed RNA genome of influenza H1N1 A/California/07/2009 using forward primer 5′-ATT CCC GTC GAC GCC ACC ATG AAG GCA ATA CTA GTA GTT CT-3′ and reverse primer 5′-ATT TAC GCC TAG GTT ATC AAA TAC ATA TTC TAC ACT GTA GAG AC-3D. The full-length H5 HA gene (H5) from A/turkey/Turkey/01/2005 (H5N1) virus strain was also amplified. Further, H7 HA gene from A/Shanghai/2/2013 was amplified from a DNA fragment, assembled based on a previously described enzymatic isothermal assembly method with error correction (Dormitzer et al, Sci Transl Med 2013, 15; 5(185):185ra68), using forward primer 5′-AAT TAA GTC GAC GCC ACC ATG AAC ACT CAA ATC CTG GTA TTC G-3′ and reverse primer 5′-AAT TAA TCT AGA TTA TCA TAT ACA AAT AGT GCA CCG CAT G-3′. Amplicons were cloned into the A836 TC83 vector shown as SEQ ID NO: [INSERT no. for TC83 sequence].

Plasmids were amplified in Escherichia coli and purified using Qiagen maxi Kits (Qiagen, Valencia, CA, USA). DNA was linearized immediately following the 3′ end of the self-amplifying RNA sequence by restriction digestion with PmeI Linearized DNA plasmids were transcribed in to RNA using MEGAscript T7 Kit (Life Technologies) and purified by LiCl precipitation. RNA was then capped using the ScriptCap m⁷G capping system (Cell Script) and purified by 2.8 M LiCl precipitation before formulation. Protein expression was confirmed by Western blot analysis of transfected baby hamster kidney (BHK) cell lysate.

### RNA Amplification and Protein Expression Analysis

RNA amplification efficiency was carried out, as previously reported (Magini et al, PLoS One 2016; 11(8):e0161193). Briefly, BHK cells were electroporated with 200 ng of RNA and incubated for overnight (15-17) hours at 37° C. and 5% CO₂. Next day, cells were collected and stained with Live/Dead Aqua (Invitrogen), APC-conjugated anti-double stranded (ds) RNA antibody (J2 monoclonal mouse antibody IgG2a kappa chain, Bioclass). Frequencies of dsRNA+ cells were analyzed by FACS CANTO II flow cytometer (BD Biosciences).

To confirm expression of HA from replicon RNA, 10⁶ BHK cells were transfected with 3 μg of each RNA using Lipofectamine 2000™ (LifeTechnologies, CA, USA). Cells were harvested 16 hours after transfection and lysed in 100 μL of 1× radio-immunoprecipitation assay (RIPA) buffer (Cell Signaling Technology, MA, USA) with complete protease inhibitor cocktail (Roche, Madison, WI, USA). Lysates were subjected to SDS-PAGE and blotted on to PVDF membrane. Protein expression was detected with HA-specific monoclonal or polyclonal antibodies.

### LNP/RNA Formulation

Equal amount of RNAs were mixed prior to encapsulation in LNPs. Formulations were characterized for particle size, RNA concentration, encapsulation efficiency and RNA integrity (using gel electrophoresis) as previously described (Hekele et al, Emerg Microbes Infect 2013). Encapsulation of RNA within LNP was carried out as described previously (Geall et al Proc Natl Acad Sci USA 2012; 109:14604-9). DLinDMA was synthesized as previously described (Heyes et al, J Control Release 107:276-287). The 1,2-Diastearoyl-sn-glycero-3-phosphocholine (DSPC) was purchased from Genzyme. Cholesterol was obtained from Sigma-Aldrich. 1,2-dimyristoyl-sn-glycero-3-phosphoethanolamine-N-[methoxy(polyethylene glycol)-2000](ammonium salt) (PEG DMG 2000) was obtained from Avanti Polar Lipids. An ethanol dilution process was used to produce the LNP formulation with the following molar ratios of lipid components: DSPC:cholesterol:PEG-DMG2000:DLinDMA 10:48:2:40 molar percent. An 8:1 N:P molar ratio (nitrogen on DlinDMA to phosphate on RNA) and 100 mM citrate buffer (pH 6) were used for the formulations. In the first step of the in-line mixing, equal volumes of lipid (in ethanol) and RNA in buffer were mixed, through a T-junction via a KDS-220 syringe pump (kdScientific), and a third syringe with equal volume of buffer was added simultaneously to the lipid/RNA mixture. After 1 h equilibration at room temperature, the mixture was further diluted with 1:1 vol/vol citrate buffer. Next, the LNPs obtained (“RV01” LNPs) were concentrated and dialyzed against 1×PBS using tangential flow filtration (TFF) (Spectrum Labs) with polyethersulfone (PES) hollow fiber membranes with a 100-kDa pore size cutoff and 20 cm² surface area. For in vitro and in vivo experiments, formulations were diluted to the required RNA concentration with 1×PBS (Teknova).

### In Vivo Models

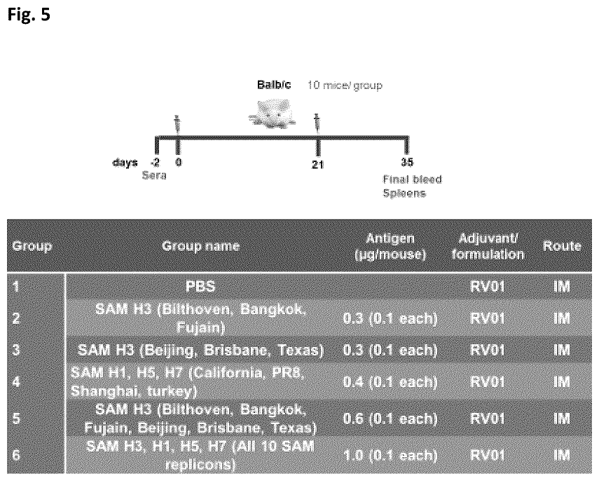

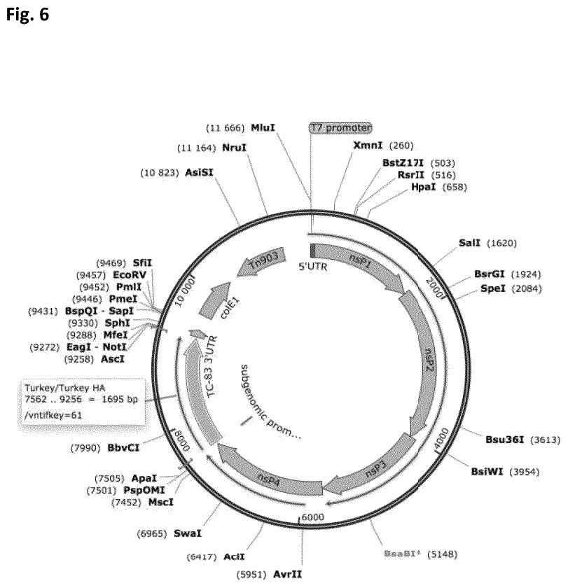

Mice were housed in the GlaxoSmithKline (GSK) Vaccines Animal Facilities, in compliance with ARRIVE guidelines and with the GSK Animal Welfare Policy and Standards. Female BALB/c mice, aged 6-8 weeks, were obtained from Charles River Laboratories, Italy. To access breadth of immune responses, groups of 10 mice were immunized intramuscularly (i.m.) with each 0.1 μg of each LNP encapsulated monocistronic or bicistronic SAM RNA separately: SAM (H1-Cal); SAM (H5-turkey); SAM (H5-H1) or in combination of groups: SAM(H1)+SAM(H5); SAM(H3-Biltho)+SAM(H3-Bang)+SAM(H3-Fuj) [group 2]; SAM(H3-Beij)+SAM(H3-Bris)+SAM(H3-Tex) [group 3]; SAM(H1-Cal)+SAM(H1-PR8)+SAM(H5-turkey)+SAM (H7-Shan) [group 4]; SAM(H3-Biltho)+SAM(H3-Bang)+SAM(H3-Fuj)+SAM(H3-Beij)+SAM(H3-Bris)+SAM(H3-Tex) [group 5] and SAM(H3-Biltho)+SAM(H3-Bang)+SAM(H3-Fuj)+SAM(H3-Beij)+SAM(H3-Bris)+SAM(H3-Tex)+SAM(H1-Cal)+SAM(H1-PR8)+SAM(H5-turkey)+SAM (H7-Shan) [group 6] twice at 3 week intervals (see FIG. 5). As control PBS was used [group 1]. Blood samples were collected 2 weeks after the second immunization for HA-specific humoral responses assessment. Spleens from 6 animals were collected form each group for evaluating CD4⁺ and CD8⁺ T cell responses.

### Hemagglutination Inhibition Assay

Serum Ab titers measured by hemagglutination inhibition (HI) assays were performed according to standard procedure using a 0.5% suspension of adult turkey erythrocytes. To inactivate nonspecific inhibitors, all serum samples were pre-treated with receptor-destroying enzymes (DENKA, Tokyo, Japan) according to manufacturer's instructions. Duplicate of individual sera were serially 2-fold diluted in V-bottom microtiter plates to achieve serum final dilutions of 1:10. Diluted sera samples were incubated with an equal volume of strain-specific influenza antigen for 60 min at room temperature followed by 60 min incubation with 0.5% turkey red blood cell suspension. The outcomes were analyzed by visual inspection and HI titres calculated as the reciprocal of the last serum dilution at which the last complete agglutination occurred.

### Intracellular Cytokine Staining

To characterize antigen-specific T-cell responses, single cell suspension of 1.5×10⁶ splenocytes were cultured with H1-Cal or H5-turkey (JPT, Berlin, Germany) or H1-PR8 (Department of Biochemistry, University of Lausanne, Switzerland) peptide pools or CD4 restricted ALNNRFQIKGVELKS (for A/Memphis/1/1971, H3N2)(Fitzmaurice et al, Vaccine 1996; 14:553-60) peptides at 2.5 μg/ml and recombinant HA proteins at 5 μg/ml concentration (Sino Biologicals Inc.) in complete RPMI media containing brefeldin A in the presence of CD107a FITC (BD Biosciences, USA). For flow cytometry analysis, cells were then stained with Live/Dead Near InfraRed (Invitrogen, USA), anti-CD62L (BD Pharmingen), fixed and permeabilized with Cytofix/Cytoperm (BD Biosciences), and further incubated with anti-CD16/CD32 Fc-block (BD Biosciences). T-cells were stained with anti-CD3-PerCp-Cy5.5, anti-CD4-BV510, anti-CD8-PE-Texas Red, anti-CD44-240 V450, anti-IFN-γ Bv785, anti-IL-2-PEcy5, anti-TNF-βV605 (All from eBiosciences). Samples were then acquired on a LSRII special order flow cytometer (BD Biosciences) and data were analyzed using FlowJo software version 9.7.4 (TreeStar). Frequencies of antigen-specific T-cells were determined after subtracting the background measured in the corresponding negative control for each cytokine.

### Statistical Analyses

All statistical analyses were performed using GraphPad Prism 5 software (GraphPad Software, La Jolla, USA). Mann-Whitney U test was used to analyze HI titers and T-cell frequencies. P value of less than 0.05 was considered significant.

### Example 2—Generation and Characterisation of SAM Vaccines Expressing One or More Influenza HA Antigens

Firstly, full length HA gene segments from H1N1 (A/California/07/2009) and H5N1 (A/turkey/Turkey/5/2005) were cloned into the TC83 alphavirus vector comprised of VEE/SINV (Venezuelan equine encephalitis-Sindbis virus) chimeric replicon containing T7 DNA polymerase promoter (Perri et al.; Journal of Virology October 2003, p 10394-10403) as monocistronic SAM (H1-Cal), SAM (H5-turkey) and bicistronic SAM (H5-H1) replicons. In the next step, 8 more monocistronic SAM (HA) replicons SAM (H1-PR8), SAM (H3-Biltho), SAM (H3-Bang), SAM (H3-Beij), SAM (H3-Fuj), SAM (H3-Bris), SAM (H3-Tex) and SAM (H7-Shan) were generated. In-vitro synthesis of RNAs from all the SAM replicons was carried out by enzymatic transcription reaction from linearized DNA. Self-amplification efficiency of RNA was measured by quantitative detection of the intracellular dsRNA in BHK cells, followed by flow cytometric analysis.

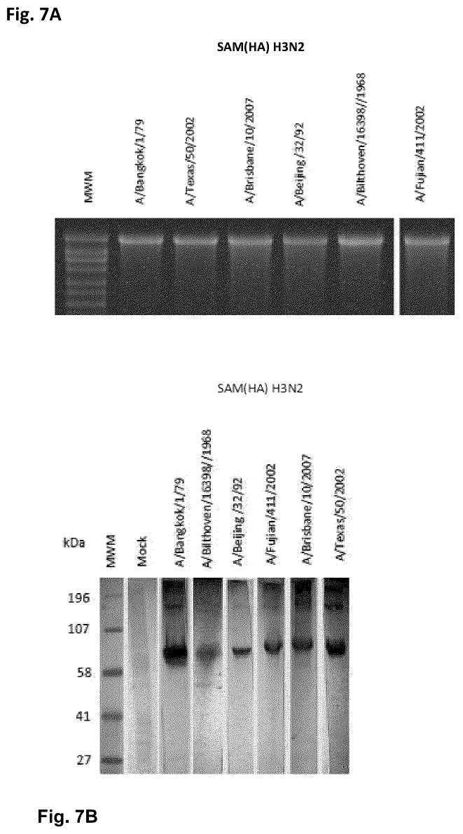

BHK cells positive for dsRNA after transfection with monocistronic and bicistronic SAM (HA) replicons were comparable with RNA of known potency. Protein expression ability of SAM replicons was tested by transfecting the BHK cells and then subjected to western blot analysis. Expression of influenza HA from H1, H5 (FIG. 1), H3 (FIG. 7), and H7 from monocistronic and bicistronic SAM (FIG. 1) replicons showed protein expression using HA-specific antibodies. Before mice immunization, SAM replicons were encapsulated in the LNP delivery system as described in Example 1. Mean particle size and polydispersity was measured by dynamic light scattering for all eleven LNP/RNA formulations. The Z average diameter of LNPs ranged from 137 to 163 nm with polydispersity index 0.01 to 0.14. Further SAM/LNP complexes were also tested for encapsulation efficiency and showed that LNPs were able to encapsulate approximately 95% of mRNA. LNP/RNA particle size and encapsulation efficiency data suggest that LNP are excellent delivery vehicles for nucleic acid delivery. Agarose gel electrophoresis showed that RNA integrity was maintained during formulation.

### Example 3—Humoral Immune Responses Following Monocistronic SAM(H1-Cal), SAM(H5-Turkey) or Bicistronic SAM (H5-H1) Vaccines

As previously mentioned, groups of 10 Balb/c mice vaccinated i.m. twice, 3 weeks apart, with 0.1 μg of SAM (H1-Cal), SAM (H5-turkey), a mixture of both SAM(H1)+SAM(H5) or SAM (H5-H1) and formulated with LNP. Serum samples were collected about 3 weeks after the first immunization and 2 weeks after the second immunization. However, only final serum samples were analyzed for antibody responses because previous results suggested that SAM vaccines can induce immune responses already at 3 weeks after a first immunization (Hekele et al, Emerg Microbes Infect, 2013; 2, e52).

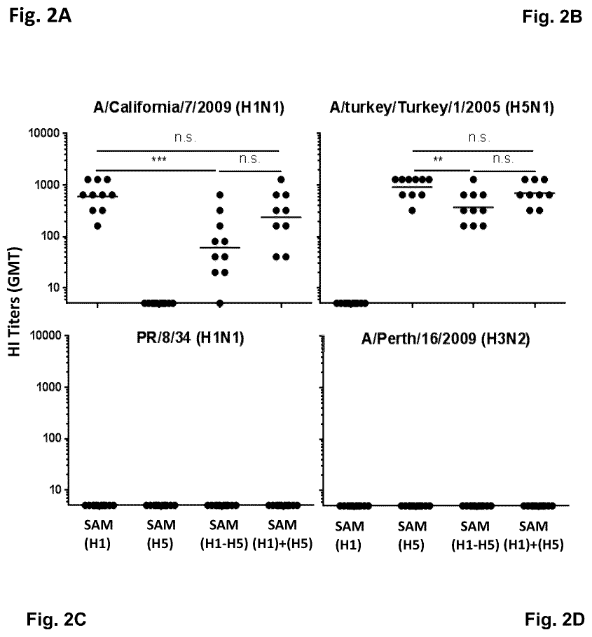

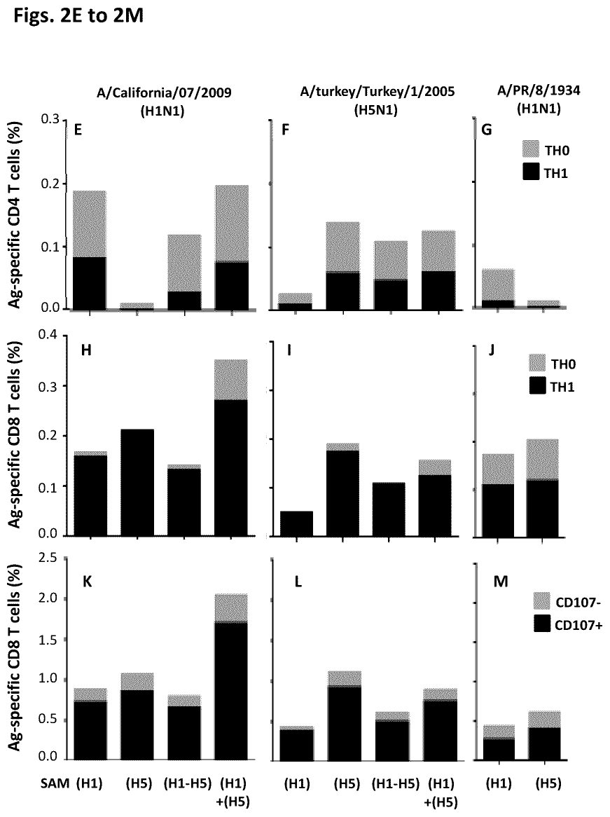

Serum samples from vaccinated Balb/c mice were tested for the presence of HA-specific functional antibodies by HI assays. Animals that received monocistronic SAM(H1-Cal)/LNP or SAM(H5-turkey)/LNP vaccine candidates developed geometric mean titer (GMT) of 597 and 905, respectively against homologous A/California/07/2009 (H1N1) and A/turkey/Turkey/5/2005 (H5N1)(FIG. 2 a & b). Interestingly, HI GMTs of bicistronic of SAM(H5-H1)/LNP appeared more than two fold lower than monocistronic vaccines against A/California/07/2009 (H1N1) and A/turkey/Turkey/5/2005 (H5N1) viruses suggesting that combinations of two different influenza antigens in a single SAM vector is not effective in boosting functional antibody responses (FIGS. 2 a & b). Moreover, the combination of SAM(H1)+SAM(H5)/LNP candidate vaccines induced comparable responses to monocistronic SAM(H1-Cal) or SAM(H5-turkey) and the difference was not statistically significant (FIGS. 2 a & b). Next, we tested whether the combination of two different antigens can induce cross-reactive functional antibodies or not. Serum from vaccinated mice subjected for HI analysis with antigenically different A/PR/8/1934 (H1N1) and A/Perth/16/2009 (H3N2) virus strains. Cross-reactivity was not observed in any of the vaccinated groups (FIGS. 2 c & d).

### Discussion

A bicistronic SAM (HA) vector expressing HA from two different influenza subtypes (H1N1 and H5N1) was developed and the ability to induce cross-reactive immune responses tested. Existing reports suggest that both subtypes induced cross-protective immunity in humans and animals (Brazzoli et al, J Virol 2015; 90:332-44, Wrammert et al, J Exp Med 2011; 208:181-93, Sridhar et al, Front Immunol 2016; 7, Florek et al, J Virol 2014; 88:13418-28). Cross-reactive antibodies against antigenically distant heterologous strains A/PR/8/1934 (H1N1) and A/Perth/16/2009), were not detected after 2 doses of bicistronic SAM(H5-H1) or monocistronic SAM(H1-Cal), SAM(H5-turkey) or combinations of SAM(H1)+SAM(H5) vaccines. Absence of antibody mediated cross-reactivity was not surprising and is consistent with the obligation for an annual update of seasonal influenza vaccines. However, the strength of the antibody responses induced by bicistronic SAM (H5-H1) was lower than SAM(H1-Cal) or SAM(H5-turkey) (<p=0.001), suggesting that individual SAM (HA) replicons expressing influenza antigens are more effective. Interestingly, cross-reactive intrasubtypic and heterosubtypic CD4⁺ and CD8⁺ were detected. The results suggest that in the absence of cross reactive neutralizing antibodies, T cells may provide protection against antigenically different viruses.

### Example 4—Cellular Immune Responses Following Monocistronic SAM(H1-Cal), SAM(H5-Turkey) or Bicistronic SAM (H5-H1) Vaccines

To characterize functional T-cell responses induced by the vaccine candidates, Balb/c mice were immunized with 0.1 μg of SAM/LNP and splenocytes were collected 2 weeks after the second vaccination and stimulated in-vitro with peptide pools covering full length HA sequence from H1-Cal and H5-turkey. The frequencies of antigen-specific CD4⁺ and CD8⁺ cytokine producing (IFN-γ, TNFα and IL-2) T-cells were analyzed by flow cytometry. All vaccine groups elicited HA-specific CD4⁺ T-cell responses with T helper (Th) cell profile Th0/Th1 phenotype (dominated by secretion of IFN-γ and combinations IFN-γ/TNF and IFN-γ/TNF/IL-2) (FIGS. 2 e, f & g). Influenza HA-specific CD8⁺ T-cells were also investigated and mostly HA-specific T-cells found positive for IFN-γ and TNF cytokines (FIGS. 2 h, l & j). Further, expression of cell surface marker CD107a was also observed, specific for degranulation associated with cytotoxic activity. A significant number of CD8⁺ T-cells were found positive for CD107a (FIGS. 2 k, l & m). Splenocytes from Balb/c mice vaccinated with SAM (H1-Cal) and SAM (H5-turkey) were stimulated with peptide pools spanning full length HA from H1/PR/8 influenza virus for determination of heterologous T-cell responses. Stimulated CD4⁺ and CD8⁺ T-cells reacted with mismatched H1-PR8 peptide pools (FIGS. 2 g, j & m) suggesting the presence of shared T-cell epitopes in HA antigens from heterologous virus strains. Cross-reactive T-cell responses against bicistronic vaccine candidates were not tested because of limitations in the amount of splenocytes.

### Example 5—Vaccination with a Cocktail of SAM Vectors Expressing HA of H3, H1, H5 and H7 Influenza Subtypes

### Induction of Functional Antibodies

In the present study, the immunogenicity of multivalent vaccines comprised of cocktails of 10 SAM replicons expressing HAs from four different influenza subtypes (H1N1, H3N2, H5N1 and H7N9) have been assessed. Cocktails of 3, 4, 6 or 10 different mRNAs (each 0.1 μg) were encapsulated in lipid nanoparticles (LNP). Groups of 10 Balb/c mice were immunized i.m. with multivalent SAM (HA) vaccine or PBS. Sera samples were collected 2 weeks after second immunization (day 35) and functional antibodies were studied by HI assays.

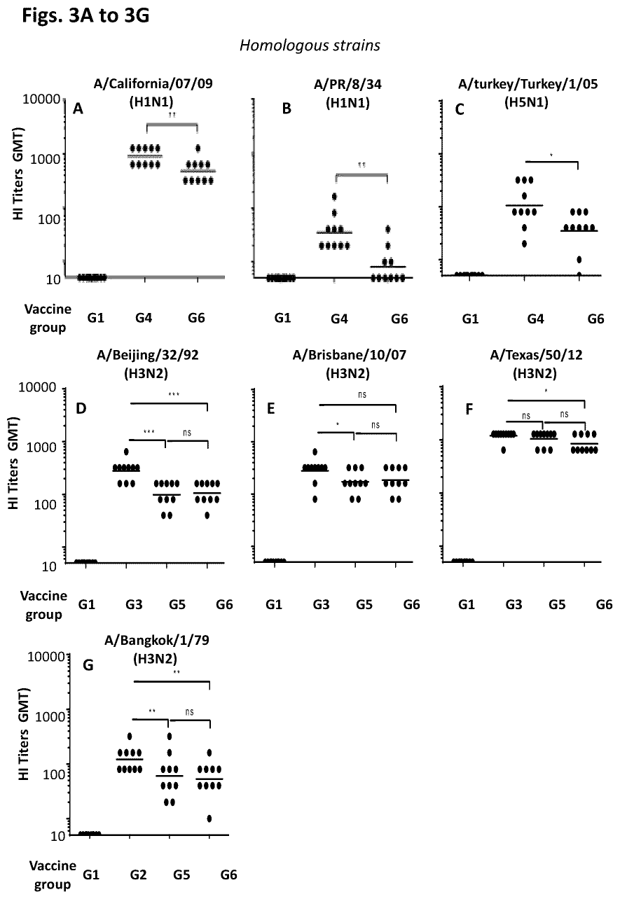

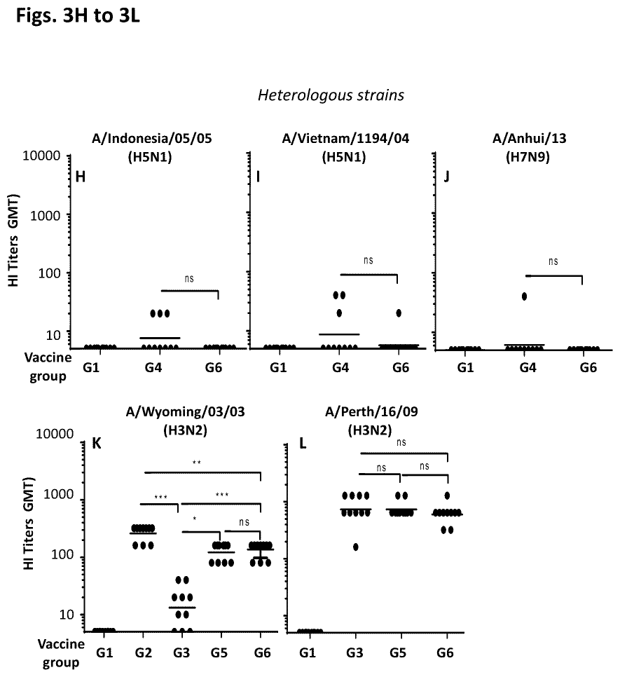

First, sera from vaccinated mice were analyzed with influenza HA viral antigens that were identical to those contained in the SAM (HA) cocktail vaccines (homologous responses) (FIG. 3 a to g). All vaccinated mice were shown to induce functional antibody responses. Functional antibody responses against the vaccine cocktail groups containing 3 SAM(HAs) (group 2 and 3) showed stronger responses than cocktail group 6 (containing 10 SAM(HAs).

To evaluate whether the multivalent vaccine could have role in protection against mismatched influenza virus, sera samples were analyzed with heterologous influenza virus antigens. Cross-reactive functional antibodies were found against all heterologous virus strains except H7N9 (A/Anhui/2013) (FIG. 3) Greater responses were observed among H3N2 subtypes. The magnitude of the HI responses against different vaccine cocktails was also analyzed. Compared to LNPs with 6 different RNAs (group 5) of the same subtypes, LNPs with 3 SAM RNAs (groups 2 and 3) boosted 1.1 to 2.7 times the antibody responses (P<0.3006) (FIG. 3 d to g). While in the presence of SAM replicons from other subtypes (group 2 and 3 Vs 6) 3 SAM replicons boosted 1.3 to 2.6 time the responses (P<0.0252) (FIGS. 3 d to g). No increase was found in LNP with 6 SAM RNAs (group 5) compared to 10 SAM RNAs (group 6). Between group 4 and 6 an increase of 1.7 to 4 times was detected (P<0.0094) (FIGS. 3a & b).

### Induction CD4⁺ Mediated Immune Responses

To determine whether multivalent SAM(HA) vaccine-induced CD4⁺ T-cell responses provide protection against mismatched influenza virus, groups of mice were vaccinated with multivalent SAM (HA) vaccine twice, 3 weeks apart. CD4⁺ T cells responses were analyzed by in-vitro stimulation of splenocytes from vaccinated Balb/c mice with full length recombinant HA proteins and peptide pools.

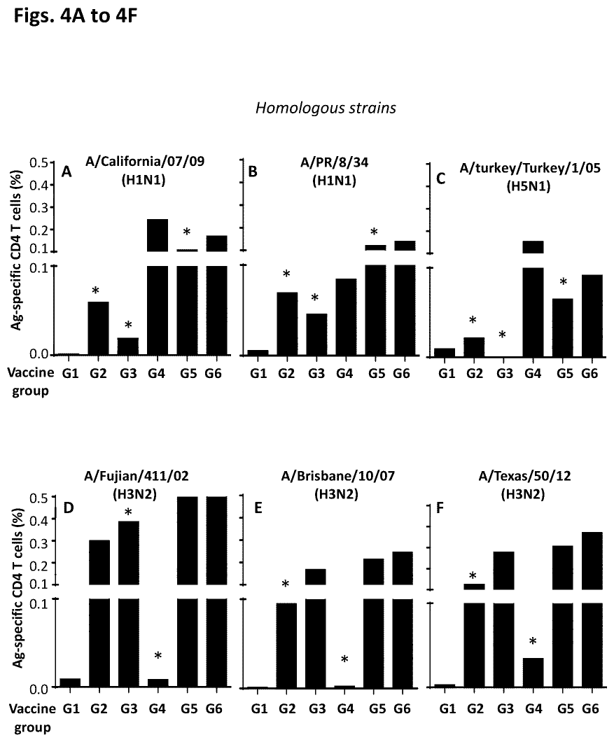

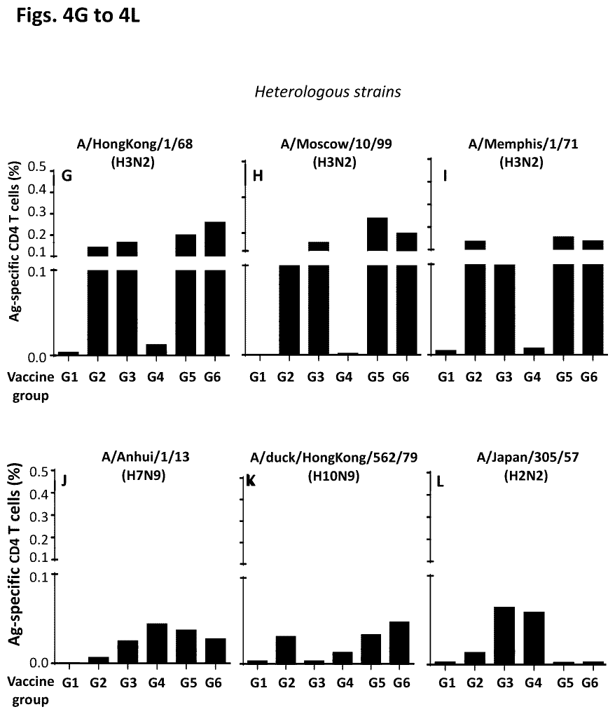

Firstly, CD4⁺ T-cell responses against HA recombinant proteins or peptides from homologous influenza viruses were tested: A/Fujian/411/2002, A/Brisbane/10/2007, A/Texas/50/2012 (H3N2), A/turkey/Turkey/1/2005 (H5N1) and A/California/07/2009, A/PR/8/1934 (H1N1). Results demonstrated CD4+ T cell responses against all homologous influenza HAs (FIG. 4 a to f). Interestingly, stimulated CD4+ T cells with peptide pools from A/California/07/2009, A/PR/8/1934 (H1N1) and A/turkey/Turkey/1/2005 (H5N1) also induced responses in other groups where antigens from those strains were not included in the SAM multivalent vaccine (FIGS. 4 a to c). To determine the cross-protection conferred by the multivalent SAM vaccines, we analyzed mice splenocytes with intrasubtypic and heterosubtypic virus strains. All SAM (HA) vaccine groups containing H3 (groups 2, 3, 5 and 6) HA induced cross reactive CD4+ T cell responses against distantly or closely related H3N2 influenza virus strains. These results suggest that intra-subtypic strains of the virus are sharing sufficient number of T cell epitopes. Furthermore, CD4+ T cell responses were detected against other subtypes from which HA antigens were not included in the SAM (HA) multivalent vaccines (FIGS. 4 k & l). However, cell frequencies of cytokine expressing CD4⁺ T cells were only <0.1%. CD4 T cell responses in group 6 with 10 SAM (HA) expressing HA antigens exhibit comparable responses to those with fewer SAM (HA) antigens suggesting the utility of a SAM vaccine platform for development of a multivalent influenza vaccine.

### Discussion

Glycosylation pattern directly contributes to virulence and is partially responsible for distinct antigenicity of influenza viruses. Glycosylation sites present on the globular head domain of HA can tolerate substitutions without compromising functionality and antibodies generated are generally strain-specific with limited breadth of cross reactivity (Gomez Lorenzo et al, Chest 2013; 143:502-10). In contrast, the HA stem domain is highly conserved across several virus strains and in many studies antibodies directed against the stem are found with cross neutralizing properties (Sun et al, J Virol, 2013; 87:8756-66 and Nabel et al, Nat Med 2010; 16:1389-91). Therefore, HA is an attractive candidate for a multivalent influenza vaccine. In the present Examples, there is a focus on a multivalent vaccine containing H3N2 subtypes because of a higher variability in glycosylation in HA antigen. Mostly strains from past and present seasonal vaccines and also pandemic strains were selected. In particular, H3 strains were selected from 1968 to 2012 to try and capture the variability seen in glycosylation sites in the head.

The experiments described herein use a cocktail strategy by combining 3, 4, 6, or 10 SAM (HA) from H1, H3, H5 and H7 subtypes for exploring cross-protective B and T-cell immune responses in a Balb/c mice model. Sequences for particular strains are shown in Table 2. Multivalent SAM (HA) vaccines were tested for the induction of functional antibodies directed against homologous and heterologous influenza virus by HI assays. Homologous and intrasubtypic antibody responses were observed (FIGS. 3 h, i, j and l) because of high amino acid homology (see Table 1 below).

TABLE 1
Amino-acid similarity (%) of HA gene segments among influenza subtypes

| | H1- | H1- | H5 | H3- | H3- | H3- | H3- | H3- | H3- | H7- | H3- | H3- |
|---|---|---|---|---|---|---|---|---|---|---|---|---|
| Strains | Cal | PR8 | turkey | Biltho | Bang | Beij | Fuj | Bris | Tex | Anh | Mem | Mos |
| H1-Cal | 100 | 82 | 62 | 43 | 43 | 44 | 42 | 43 | 42 | 41 | 43 | 42 |
| H1-PR8 | 82 | 100 | 65 | 42 | 42 | 42 | 41 | 41 | 41 | 41 | 43 | 41 |
| H5-turkey | 62 | 63 | 100 | 41 | 40 | 40 | 39 | 39 | 40 | 41 | 40 | 40 |
| H3-Biltho | 43 | 42 | 41 | 100 | 92 | 90 | 87 | 86 | 85 | 48 | 98 | 89 |
| H3-Bang | 43 | 42 | 40 | 92 | 100 | 95 | 90 | 90 | 89 | 47 | 93 | 92 |
| H3-Beij | 44 | 42 | 40 | 90 | 95 | 100 | 93 | 92 | 91 | 47 | 90 | 95 |
| H3-Fuj | 42 | 41 | 39 | 87 | 90 | 93 | 100 | 96 | 95 | 46 | 87 | 95 |
| H3-Bris | 43 | 41 | 39 | 86 | 90 | 92 | 96 | 100 | 97 | 46 | 87 | 93 |
| H3-Tex | 42 | 42 | 40 | 85 | 89 | 91 | 95 | 97 | 100 | 46 | 86 | 92 |
| H7-Anh | 41 | 41 | 41 | 48 | 47 | 47 | 46 | 46 | 46 | 100 | 47 | 47 |
| H3-Mem | 43 | 43 | 40 | 98 | 93 | 90 | 87 | 87 | 86 | 47 | 100 | 47 |
| H3-Mos | 42 | 41 | 40 | 89 | 92 | 95 | 95 | 93 | 92 | 47 | 89 | 100 |
| H2-Jap | 63 | 66 | 76 | 42 | 40 | 41 | 40 | 40 | 40 | 40 | 42 | 40 |
| H10-Ho | 43 | 43 | 42 | 52 | 51 | 51 | 49 | 50 | 49 | 65 | 52 | 50 |

ClustalW (EMBL-EBI) tool was used for amino acid similarity search for full length HA gene sequences of H1-Cal (A/California/07/2009); H1-PR8 (A/PR/8/1934); H5-turkey (A/turkey/Turkey/5/2005); H3-Biltho (A/Bilthoven/16398/1968); H3-Bang (A/Bangkok/1/79); H3-Beij (A/Beijing/32/92); H3-Fuj (A/Fujian/411/2002); H3-Bris (A/Brisbane/10/2007); H3-Tex (A/Texas/50/2012); H7-Anh (A/Anhui/1/2013); H-3-Mem (A/Memphis/1/1971); H-3-Mos (A/Moscow/10/1999); H-2-Jap (A/Japan/305/1957); H10-Ho (A/duck/Hongkong/562/1979).

TABLE 2

| Strains | EPI-Segment-ID* |
|---|---|
| A/California/07/2009 (H1N1) | EPI516528 |
| A/PR/8/34 (H1N1) | EPI131282 |
| A/turkey/Turkey/1/2005 (H5N1) | EPI102703 |
| A/Bilthoven/16398/1968 (H3N2) | EPI362379 |
| A/Bangkok/1/79 (H3N2) | EPI367158 |
| A/Beijing/32/92 (H3N2) | EPI365898 |
| A/Fujain/411/2002 (H3N2) | EPI362915 |
| A/Brisbane/10/2007 (H3N2) | EPI362338 |
| A/Texas/50/2012 (H3N2) | EPI398417 |
| A/Anhui/1/2013 (H7N9) | EPI539507 |
| A/Memphis/1/1971 (H3N2) | EPI137302 |
| A/Moscow/10/1999 (H3N2) | EPI103359 |
| A/Japan/305/1957 (H2N2) | EPI240974 |
| A/duck/Hongkong/562/1979 (H10N9) | EPI42118 |

sequences obtained from Global Initiative on Sharing All Influenza Data Epiflu database (www.gisaid.org)

Despite the absence of cross-reactive functional antibodies against heterotypic virus strains, vaccination with SAM (HA) H3N2 multivalent vaccines showed cross protective CD4⁺ T-cells against A/PR/8/1934 (H1N1), A/California/07/2009 (H1N1) and A/turkey/turkey/2005 (H5N1) (FIG. 4 a to c). Heterotypic CD4⁺ T cell responses were also detected against H7N9, H10N9 and H2N2 but to a lesser extent.

In this study, an alternative vaccine platform technology, based on SAM technology, is used to deliver multiple influenza antigens simultaneously and induce protective immune responses. This vaccine platform technology can induce a broad spectrum of immune responses and deliver multiple influenza antigens simultaneously without compromising antiginicity. This technology might be beneficial for the development of a universal influenza vaccine.

### Clauses

Clause 1. An immunogenic composition comprising: (i) a first self-replicating RNA molecule encoding a polypeptide comprising a first antigen and (ii) a second self-replicating RNA molecule encoding a polypeptide comprising a second antigen, wherein the first and second antigens are both from influenza virus but the first antigen is from a different strain of influenza virus to the second antigen.

Clause 2. The immunogenic composition of clause 1 wherein the first and second antigens are hemagglutinin (HA).

Clause 3. The immunogenic composition of clause 1 wherein the first and second antigens are an immunogenic fragment or variant of hemagglutinin (HA).

Clause 4. The immunogenic composition of any preceding clause wherein the first antigen is from a different subtype of influenza virus to the second antigen.

Clause 5. The immunogenic composition of any preceding clause wherein the first and second antigens are the only antigens derived from influenza virus in the self-replicating RNA molecules.

Clause 6. The immunogenic composition of any preceding clause further comprising: (iii) a third self-replicating RNA molecule encoding a polypeptide comprising a third antigen, wherein the third antigen is from influenza virus but is from a different strain of influenza virus to both the first and second antigens.

Clause 7. The immunogenic composition of clause 6 wherein the first, second and third antigens are the only antigens derived from influenza virus in the self-replicating RNA molecules.

Clause 8. The immunogenic composition of clause 6 or 7 further comprising: (iii) a fourth self-replicating RNA molecule encoding a polypeptide comprising a fourth antigen, wherein the fourth antigen is from influenza virus but is from a different strain of influenza virus to both the first, second and third antigens.

Clause 9. The immunogenic composition of any preceding clause wherein the first antigen is from influenza A subtype H1 and the second antigen is from a different H1 strain to the first antigen.

Clause 10. The immunogenic composition of any one of clauses 1 to 8 wherein the first and second antigens are from influenza A subtype H3, and wherein both antigens are derived from different strains of H3 influenza virus.

Clause 11. The immunogenic composition of clause 8 wherein the first and second antigens are from influenza A subtype H1 and the third and fourth antigens are from influenza A subtype H3, and wherein the first and second antigens are derived from different strains of H1 virus and the third and fourth antigens are from different strains of H3 influenza virus.

Clause 12. The immunogenic composition of clauses 9 or 11 wherein the antigens are from influenza subtype H1N1.

Clause 13. The immunogenic composition of clauses 10 or 11 wherein the antigens are from influenza subtype H3N2.

Clause 14. The immunogenic composition of any of clauses 8 to 13 further comprising one or more further self-replicating RNA molecules, wherein each further self-replicating RNA molecules encodes a polypeptide comprising a further antigen, wherein each further antigen is from influenza virus but is from a different strain of influenza virus to any of the other antigens in the composition.

Clause 15. The immunogenic composition of clause 14 wherein the composition comprises 5, 6, 7, 8, 9 or 10 further self-replicating RNA molecules.

Clause 16. The immunogenic composition of any preceding clause wherein the composition comprises six self-replicating RNA molecules, wherein each self-replicating RNA molecules encodes a polypeptide comprising an antigen from a different strain of influenza subtype H3N2.

Clause 17. The immunogenic composition of any preceding clause further comprising an adjuvant.

Clause 18. The immunogenic composition of any preceding clause wherein the self-replicating RNA molecule is a derived from an alphavirus.

Clause 19. The immunogenic composition of clause 18 wherein the alphavirus is selected from the group consisting of: Sindbis (SIN), Venezuelan equine encephalitis (VEE), Semliki Forest virus (SFV) and combinations thereof.

Clause 20. The immunogenic composition of any preceding clause wherein the self-replicating RNA molecules are monocistronic.

Clause 21. A pharmaceutical composition comprising an immunogenic composition according to any one of the preceding clauses and a pharmaceutically acceptable carrier.

Clause 22. The pharmaceutical composition of clause 21 further comprising a cationic lipid, a liposome, a microparticle, viral replicon particles (VRPs), an oil-in-water emulsion or a cationic nanoemulsion.

Clause 23. The pharmaceutical composition of clause 23 wherein the self-replicating RNA molecules are encapsulated in, bound to or adsorbed on a cationic lipid, a liposome, a microparticle, viral replicon particles (VRPs), an oil-in-water emulsion or a cationic nanoemulsion.

Clause 24. The immunogenic composition of any one of clauses 1 to 20 or the pharmaceutical composition of any one of clauses 21 to 23 wherein the self-replicating RNA molecules are formulated in lipid nanoparticles (LNP) or in a cationic nanoemulsion (CNE).

Clause 25. The immunogenic composition of any one of clauses 1 to 20 or the pharmaceutical composition of any one of clauses 21 to 23 for use as a vaccine.

Clause 26. The immunogenic composition of any one of clauses 1 to 20 or the pharmaceutical composition of any one of clauses 21 to 23 for use in the prevention of influenza.

Clause 27. A method of prevention and/or treatment against influenza disease, comprising the administration of an immunogenic composition according to any one of clauses 1 to 20 or the pharmaceutical composition of any one of clauses 21 to 23 to a person in need thereof.

Clause 28. A method for inducing an immune response in a subject comprising administering to the subject an effective amount of a pharmaceutical composition according to any one of clauses 21 to 23 or the immunogenic composition of any one of clauses 1 to 20.

Clause 29. A method of prevention and/or treatment against influenza disease, comprising (i) the administration of a first immunogenic composition comprising a first self-replicating RNA molecule and pharmaceutically acceptable carrier and (ii) simultaneous or sequential administration of a second immunogenic composition comprising a second self-replicating RNA molecule and pharmaceutically acceptable carrier, wherein the first and second self-replicating RNA molecules encode a polypeptide encoding an antigen from influenza virus but the first self-replicating RNA molecule encodes an antigen from a different strain of influenza to that encoded by the second self-replicating RNA molecule.

Clause 30. A first immunogenic composition comprising a first self-replicating RNA molecule and a pharmaceutically acceptable carrier for use in a method of preventing influenza disease, said method comprising administration to a subject in need the first immunogenic composition followed by administration of a second immunogenic composition comprising a self-replicating RNA molecule and a pharmaceutically acceptable carrier, wherein the first and second self-replicating RNA molecules each encode a polypeptide comprising an antigen, wherein the antigen is from influenza virus but the antigen encoded by the first self-replicating RNA molecule is from a different strain of influenza virus to that encoded by the second self-replicating RNA molecule.

Clause 31. The immunogenic composition for use according to clause 30 wherein the first and second immunogenic compositions are administered simultaneously, at substantially the same time or sequentially.

Clause 32. The immunogenic composition for use according to clause 30 wherein the first and second immunogenic compositions are administered sequentially with an interval of less than 10 hours, from 1 second to 10 hours or from 1 second to 1 hour.

Clause 33. A method of preparing an immunogenic composition according to any one of clauses 1 to 20 or 24, the method comprising: (i) providing an oil-in-water emulsion; (ii) providing an aqueous solution comprising the self-replicating RNA molecules; and (iii) combining the aqueous solution of (ii) and the oil-in-water emulsion of (i), thereby preparing the composition.

Clause 34. A method of preparing an immunogenic composition according to any one of clauses 1 to 20 or 24, the method comprising: (i) providing at least one lipid which forms nanoparticles; (ii) providing an aqueous solution comprising the self-replicating RNA molecules; and (iii) combining the aqueous solution of (ii) and the at least one lipid of (i), thereby preparing the composition.

SEQUENCE LISTINGS

SEQ ID NO: 1: Nucleotide cDNA sequence of A836 TC83 VEE/SINV (Venezuelan equine encephalitis-
Sindbis virus) chimeric replicon containing T7 DNA polymerase promoter
ataggcggcgcatgagagaagcccagaccaattacctacccaaaatggagaaagttcacgttgacatcgaggaagacagcccattcctcag
agctttgcagcggagcttcccgcagtttgaggtagaagccaagcaggtcactgataatgaccatgctaatgccagagcgttttcgcatctggctt
caaaactgatcgaaacggaggtggacccatccgacacgatccttgacattggaagtgcgcccgcccgcagaatgtattctaagcacaagtatc
attgtatctgtccgatgagatgtgcggaagatccggacagattgtataagtatgcaactaagctgaagaaaaactgtaaggaaataactgata
aggaattggacaagaaaatgaaggagctcgccgccgtcatgagcgaccctgacctggaaactgagactatgtgcctccacgacgacgagtc
gtgtcgctacgaagggcaagtcgctgtttaccaggatgtatacgcggttgacggaccgacaagtctctatcaccaagccaataagggagttag
agtcgcctactggataggctttgacaccaccccttttatgtttaagaacttggctggagcatatccatcatactctaccaactgggccgacgaaac
cgtgttaacggctcgtaacataggcctatgcagctctgacgttatggagcggtcacgtagagggatgtccattcttagaaagaagtatttgaaa
ccatccaacaatgttctattctctgttggctcgaccatctaccacgagaagagggacttactgaggagctggcacctgccgtctgtatttcacttac
gtggcaagcaaaattacacatgtcggtgtgagactatagttagttgcgacgggtacgtcgttaaaagaatagctatcagtccaggcctgtatgg
gaagccttcaggctatgctgctacgatgcaccgcgagggattcttgtgctgcaaagtgacagacacattgaacggggagagggtctcttttccc
gtgtgcacgtatgtgccagctacattgtgtgaccaaatgactggcatactggcaacagatgtcagtgcggacgacgcgcaaaaactgctggtt
gggctcaaccagcgtatagtcgtcaacggtcgcacccagagaaacaccaataccatgaaaaattaccttttgcccgtagtggcccaggcattt
gctaggtgggcaaaggaatataaggaagatcaagaagatgaaaggccactaggactacgagatagacagttagtcatggggtgttgttgg
gcttttagaaggcacaagataacatctatttataagcgcccggatacccaaaccatcatcaaagtgaacagcgatttccactcattcgtgctgcc
caggataggcagtaacacattggagatcgggctgagaacaagaatcaggaaaatgttagaggagcacaaggagccgtcacctctcattacc
gccgaggacgtacaagaagctaagtgcgcagccgatgaggctaaggaggtgcgtgaagccgaggagttgcgcgcagctctaccacctttg
gcagctgatgttgaggagcccactctggaagccgatgtcgacttgatgttacaagaggctggggccggctcagtggagacacctcgtggcttg
ataaaggttaccagctacgatggcgaggacaagatcggctcttacgctgtgctttctccgcaggctgtactcaagagtgaaaaattatcttgcat
ccaccctctcgctgaacaagtcatagtgataacacactctggccgaaaagggcgttatgccgtggaaccataccatggtaaagtagtggtgcc
agagggacatgcaatacccgtccaggactttcaagctctgagtgaaagtgccaccattgtgtacaacgaacgtgagttcgtaaacaggtacct
gcaccatattgccacacatggaggagcgctgaacactgatgaagaatattacaaaactgtcaagcccagcgagcacgacggcgaatacctgt
acgacatcgacaggaaacagtgcgtcaagaaagaactagtcactgggctagggctcacaggcgagctggtggatcctcccttccatgaattc
gcctacgagagtctgagaacacgaccagccgctccttaccaagtaccaaccataggggtgtatggcgtgccaggatcaggcaagtctggcat
cattaaaagcgcagtcaccaaaaaagatctagtggtgagcgccaagaaagaaaactgtgcagaaattataagggacgtcaagaaaatgaa
agggctggacgtcaatgccagaactgtggactcagtgctcttgaatggatgcaaacaccccgtagagaccctgtatattgacgaagcttttgct
tgtcatgcaggtactctcagagcgctcatagccattataagacctaaaaaggcagtgctctgcggggatcccaaacagtgcggtttttttaacat
gatgtgcctgaaagtgcattttaaccacgagatttgcacacaagtcttccacaaaagcatctctcgccgttgcactaaatctgtgacttcggtcgt
ctcaaccttgttttacgacaaaaaaatgagaacgacgaatccgaaagagactaagattgtgattgacactaccggcagtaccaaacctaagca
ggacgatctcattctcacttgtttcagagggtgggtgaagcagttgcaaatagattacaaaggcaacgaaataatgacggcagctgcctctca
agggctgacccgtaaaggtgtgtatgccgttcggtacaaggtgaatgaaaatcctctgtacgcacccacctcagaacatgtgaacgtcctactg
acccgcacggaggaccgcatcgtgtggaaaacactagccggcgacccatggataaaaacactgactgccaagtaccctgggaatttcactgc
cacgatagaggagtggcaagcagagcatgatgccatcatgaggcacatcttggagagaccggaccctaccgacgtcttccagaataaggca
aacgtgtgttgggccaaggctttagtgccggtgctgaagaccgctggcatagacatgaccactgaacaatggaacactgtggattattttgaaa
cggacaaagctcactcagcagagatagtattgaaccaactatgcgtgaggttctttggactcgatctggactccggtctattttctgcacccactg
ttccgttatccattaggaataatcactgggataactccccgtcgcctaacatgtacgggctgaataaagaagtggtccgtcagctctctcgcagg
tacccacaactgcctcgggcagttgccactggaagagtctatgacatgaacactggtacactgcgcaattatgatccgcgcataaacctagtac
ctgtaaacagaagactgcctcatgctttagtcctccaccataatgaacacccacagagtgacttttcttcattcgtcagcaaattgaagggcaga
actgtcctggtggtcggggaaaagttgtccgtcccaggcaaaatggttgactggttgtcagaccggcctgaggctaccttcagagctcggctg
gatttaggcatcccaggtgatgtgcccaaatatgacataatatttgttaatgtgaggaccccatataaataccatcactatcagcagtgtgaaga
ccatgccattaagcttagcatgttgaccaagaaagcttgtctgcatctgaatcccggcggaacctgtgtcagcataggttatggttacgctgaca
gggccagcgaaagcatcattggtgctatagcgcggcagttcaagttttcccgggtatgcaaaccgaaatcctcacttgaagagacggaagttc
tgtttgtattcattgggtacgatcgcaaggcccgtacgcacaatccttacaagctttcatcaaccttgaccaacatttatacaggttccagactcca
cgaagccggatgtgcaccctcatatcatgtggtgcgaggggatattgccacggccaccgaaggagtgattataaatgctgctaacagcaaag
gacaacctggcggaggggtgtgcggagcgctgtataagaaattcccggaaagcttcgatttacagccgatcgaagtaggaaaagcgcgact
ggtcaaaggtgcagctaaacatatcattcatgccgtaggaccaaacttcaacaaagtttcggaggttgaaggtgacaaacagttggcagagg
cttatgagtccatcgctaagattgtcaacgataacaattacaagtcagtagcgattccactgttgtccaccggcatcttttccgggaacaaagatc
gactaacccaatcattgaaccatttgctgacagctttagacaccactgatgcagatgtagccatatactgcagggacaagaaatgggaaatga
ctctcaaggaagcagtggctaggagagaagcagtggaggagatatgcatatccgacgactcttcagtgacagaacctgatgcagagctggt
gagggtgcatccgaagagttctttggctggaaggaagggctacagcacaagcgatggcaaaactttctcatatttggaagggaccaagtttca
ccaggcggccaaggatatagcagaaattaatgccatgtggcccgttgcaacggaggccaatgagcaggtatgcatgtatatcctcggagaaa
gcatgagcagtattaggtcgaaatgccccgtcgaagagtcggaagcctccacaccacctagcacgctgccttgcttgtgcatccatgccatgac
tccagaaagagtacagcgcctaaaagcctcacgtccagaacaaattactgtgtgctcatcctttccattgccgaagtatagaatcactggtgtgc
agaagatccaatgctcccagcctatattgttctcaccgaaagtgcctgcgtatattcatccaaggaagtatctcgtggaaacaccaccggtagac
gagactccggagccatcggcagagaaccaatccacagaggggacacctgaacaaccaccacttataaccgaggatgagaccaggactaga
acgcctgagccgatcatcatcgaagaggaagaagaggatagcataagtttgctgtcagatggcccgacccaccaggtgctgcaagtcgagg
cagacattcacgggccgccctctgtatctagctcatcctggtccattcctcatgcatccgactttgatgtggacagtttatccatacttgacaccctg
gagggagctagcgtgaccagcggggcaacgtcagccgagactaactcttacttcgcaaagagtatggagtttctggcgcgaccggtgcctgc
gcctcgaacagtattcaggaaccctccacatcccgctccgcgcacaagaacaccgtcacttgcacccagcagggcctgctcgagaaccagcct
agtttccaccccgccaggcgtgaatagggtgatcactagagaggagctcgaggcgcttaccccgtcacgcactcctagcaggtcggtctcgag
aaccagcctggtctccaacccgccaggcgtaaatagggtgattacaagagaggagtttgaggcgttcgtagcacaacaacaatgacggtttg
atgcgggtgcatacatcttttcctccgacaccggtcaagggcatttacaacaaaaatcagtaaggcaaacggtgctatccgaagtggtgttgga
gaggaccgaattggagatttcgtatgccccgcgcctcgaccaagaaaaagaagaattactacgcaagaaattacagttaaatcccacacctg
ctaacagaagcagataccagtccaggaaggtggagaacatgaaagccataacagctagacgtattctgcaaggcctagggcattatttgaa
ggcagaaggaaaagtggagtgctaccgaaccctgcatcctgttcctttgtattcatctagtgtgaaccgtgccttttcaagccccaaggtcgcag
tggaagcctgtaacgccatgttgaaagagaactttccgactgtggcttcttactgtattattccagagtacgatgcctatttggacatggttgacg
gagcttcatgctgcttagacactgccagtttttgccctgcaaagctgcgcagctttccaaagaaacactcctatttggaacccacaatacgatcg
gcagtgccttcagcgatccagaacacgctccagaacgtcctggcagctgccacaaaaagaaattgcaatgtcacgcaaatgagagaattgcc
cgtattggattcggcggcctttaatgtggaatgcttcaagaaatatgcgtgtaataatgaatattgggaaacgtttaaagaaaaccccatcagg
cttactgaagaaaacgtggtaaattacattaccaaattaaaaggaccaaaagctgctgctctttttgcgaagacacataatttgaatatgttgca
ggacataccaatggacaggtttgtaatggacttaaagagagacgtgaaagtgactccaggaacaaaacatactgaagaacggcccaaggta
caggtgatccaggctgccgatccgctagcaacagcgtatctgtgcggaatccaccgagagctggttaggagattaaatgcggtcctgcttccg
aacattcatacactgtttgatatgtcggctgaagactttgacgctattatagccgagcacttccagcctggggattgtgttctggaaactgacatc
gcgtcgtttgataaaagtgaggacgacgccatggctctgaccgcgttaatgattctggaagacttaggtgtggacgcagagctgttgacgctg
attgaggcggctttcggcgaaatttcatcaatacatttgcccactaaaactaaatttaaattcggagccatgatgaaatctggaatgttcctcaca
ctgtttgtgaacacagtcattaacattgtaatcgcaagcagagtgttgagagaacggctaaccggatcaccatgtgcagcattcattggagatg
acaatatcgtgaaaggagtcaaatcggacaaattaatggcagacaggtgcgccacctggttgaatatggaagtcaagattatagatgctgtg
gtgggcgagaaagcgccttatttctgtggagggtttattttgtgtgactccgtgaccggcacagcgtgccgtgtggcagaccccctaaaaaggc
tgtttaagcttggcaaacctctggcagcagacgatgaacatgatgatgacaggagaagggcattgcatgaagagtcaacacgctggaaccg
agtgggtattctttcagagctgtgcaaggcagtagaatcaaggtatgaaaccgtaggaacttccatcatagttatggccatgactactctagcta
gcagtgttaaatcattcagctacctgagaggggcccctataactctctacggctaacctgaatggactacgacatagtctagtccgccaagatg
gagaaaatagtgcttcttcttgcaatagtcagccttgttaaaagtgatcagatttgcattggttaccatgcaaacaactcgacagagcaggttga
cacaataatggaaaagaacgtcactgttacacacgcccaagacatactggaaaagacacacaacgggaaactctgcgatctagatggagtg
aagcctctaattttaagagattgtagtgtagctggatggctcctcgggaacccaatgtgtgacgaattcctcaatgtgccggaatggtcttacata
gtggagaagatcaatccagccaatgacctctgttacccagggaatttcaacgactatgaagaactgaaacacctattgagcagaataaaccat
tttgagaaaattcagatcatccccaaaagttcttggtcagatcatgaagcctcagcaggggtgagctcagcatgtccataccagggaaggtcct
ccttttttagaaatgtggtatggcttatcaaaaaggacaatgcatacccaacaataaagagaagttacaataataccaaccaagaagatcttttg
gtattgtgggggattcaccatccaaatgatgcggcagagcagacaaggctctatcaaaacccaactacctatatttccgttgggacatcaacac
taaaccagagattggtaccaaaaatagccactagatctaaggtaaacgggcaaagtggaaggatggagttcttttggacaattttaaaaccga
atgatgcaataaactttgagagtaatggaaatttcattgctccagaaaatgcatacaaaattgtcaagaaaggggactcaacaattatgaaaa
gtgagttggaatatggtaactgcaacaccaagtgtcaaactccaataggggcgataaactctagtatgccattccacaacatccaccctctcac
catcggggaatgccccaaatatgtgaaatcaagcagattagtccttgctactgggctcagaaatagccctcaacgagagacacgaggactatt
tggagctatagcaggttttatagagggaggatggcagggaatggtagatggttggtatgggtaccaccatagcaacgagcaggggagtggg
tacgctgcagacaaagaatccactcaaaaggcaatagatggagtcaccaataaggtcaactcgatcattgacaaaatgaacactcagtttga
ggctgttggaagggaatttaataacttagaaaggagaatagaaaatttaaacaagaagatggaagacggattcctagatgtctggacttata
atgctgaacttctggttctcatggaaaatgagagaactctagactttcatgactcaaatgtcaagaacctttacgacaaggtccgactacagctt
agggataatgcaaaggagcttggtaacggttgtttcgagttctatcacagatgtgataatgaatgtatggaaagtgtaagaaacggaacgtat
gactacccgcagtattcagaagaagcaagattaaaaagagaggaaataagtggagtaaaattggaatcaataggaacttaccaaatactgt
caatttattcaacagtggcgagctccctagcactggcaatcatggtggctggtctatctttatggatgtgctccaatggatcgttacaatgcagaa
tttgcatttaaggcgcgcccacccagcggccgcatacagcagcaattggcaagctgcttacatagaactcgcggcgattggcatgccgccttaa
aatttttattttatttttcttttcttttccgaatcggattttgtttttaatatttcaaaaaaaaaaaaaaaaaaaaaaaaaaaaaaaaaaaaaaaaga
agagcgtttaaacacgtgatatctggcctcatgggccttcctttcactgcccgctttccagtcgggaaacctgtcgtgccagctgcattaacatgg
tcatagctgtttccttgcgtattgggcgctctccgcttcctcgctcactgactcgctgcgctcggtcgttcgggtaaagcctggggtgcctaatgag
caaaaggccagcaaaaggccaggaaccgtaaaaaggccgcgttgctggcgtttttccataggctccgcccccctgacgagcatcacaaaaat
cgacgctcaagtcagaggtggcgaaacccgacaggactataaagataccaggcgtttccccctggaagctccctcgtgcgctctcctgttccg
accctgccgcttaccggatacctgtccgcctttctcccttcgggaagcgtggcgctttctcatagctcacgctgtaggtatctcagttcggtgtagg
tcgttcgctccaagctgggctgtgtgcacgaaccccccgttcagcccgaccgctgcgccttatccggtaactatcgtcttgagtccaacccggta
agacacgacttatcgccactggcagcagccactggtaacaggattagcagagcgaggtatgtaggcggtgctacagagttcttgaagtggtg
gcctaactacggctacactagaagaacagtatttggtatctgcgctctgctgaagccagttaccttcggaaaaagagttggtagctcttgatccg
gcaaacaaaccaccgctggtagcggtggtttttttgtttgcaagcagcagattacgcgcagaaaaaaaggatctcaagaagatcctttgatcttt
tctacggggtctgacgctcagtggaacgaaaactcacgttaagggattttggtcatgaatacacggtgcctgactgcgttagcaatttaactgtg
ataaactaccgcattaaagcttatcgatgataagctgtcaaacatgagaattcttagaaaaactcatcgagcatcaaatgaaactgcaatttatt
catatcaggattatcaataccatatttttgaaaaagccgtttctgtaatgaaggagaaaactcaccgaggcagttccataggatggcaagatcct
ggtatcggtctgcgattccgactcgtccaacatcaatacaacctattaatttcccctcgtcaaaaataaggttatcaagtgagaaatcaccatgag
tgacgactgaatccggtgagaatggcaaaagcttatgcatttctttccagacttgttcaacaggccagccattacgctcgtcatcaaaatcactc
gcatcaaccaaaccgttattcattcgtgattgcgcctgagcgagacgaaatacgcgatcgctgttaaaaggacaattacaaacaggaatcgaa
tgcaaccggcgcaggaacactgccagcgcatcaacaatattttcacctgaatcaggatattcttctaatacctggaatgctgttttcccggggatc
gcagtggtgagtaaccatgcatcatcaggagtacggataaaatgcttgatggtcggaagaggcataaattccgtcagccagtttagtctgacc
atctcatctgtaacatcattggcaacgctacctttgccatgtttcagaaacaactctggcgcatcgggcttcccatacaatcgatagattgtcgcac
ctgattgcccgacattatcgcgagcccatttatacccatataaatcagcatccatgttggaatttaatcgcggcctcgagcaagacgtttcccgtt
gaatatggctcataacaccccttgtattactgtttatgtaagcagacagttttattgttcatgagcggatacatatttgaatgtatttagaaaaataa
acaaataggggttccgcgcacatttccccgaaaagtgccacctaaattgtaagcgttaatattttgttaaaattcgcgttaaatttttgttaaatca
gctcattttttaaccaataggccgaaatcggcaaaatcccttataaatcaaaagaatagaccgagatagggttgagtggccgctacagggcgc
tcccattcgccattcaggctgcgcaactgttgggaagggcgtttcggtgcgggcctcttcgctattacgccagctggcgaaagggggatgtgct
gcaaggcgattaagttgggtaacgccagggttttcccagtcacacgcgtaatacgactcactatag
SEQ ID NO: 2: Nucleotide cDNA sequence of A836 TC83 VEE/SINV chimeric replicon without insert
ataggcggcgcatgagagaagcccagaccaattacctacccaaaatggagaaagttcacgttgacatcgaggaagacagcccattcctcag
agctttgcagcggagcttcccgcagtttgaggtagaagccaagcaggtcactgataatgaccatgctaatgccagagcgttttcgcatctggctt
caaaactgatcgaaacggaggtggacccatccgacacgatccttgacattggaagtgcgcccgcccgcagaatgtattctaagcacaagtatc
attgtatctgtccgatgagatgtgcggaagatccggacagattgtataagtatgcaactaagctgaagaaaaactgtaaggaaataactgata
aggaattggacaagaaaatgaaggagctcgccgccgtcatgagcgaccctgacctggaaactgagactatgtgcctccacgacgacgagtc
gtgtcgctacgaagggcaagtcgctgtttaccaggatgtatacgcggttgacggaccgacaagtctctatcaccaagccaataagggagttag
agtcgcctactggataggctttgacaccaccccttttatgtttaagaacttggctggagcatatccatcatactctaccaactgggccgacgaaac
cgtgttaacggctcgtaacataggcctatgcagctctgacgttatggagcggtcacgtagagggatgtccattcttagaaagaagtatttgaaa
ccatccaacaatgttctattctctgttggctcgaccatctaccacgagaagagggacttactgaggagctggcacctgccgtctgtatttcacttac
gtggcaagcaaaattacacatgtcggtgtgagactatagttagttgcgacgggtacgtcgttaaaagaatagctatcagtccaggcctgtatgg
gaagccttcaggctatgctgctacgatgcaccgcgagggattcttgtgctgcaaagtgacagacacattgaacggggagagggtctcttttccc
gtgtgcacgtatgtgccagctacattgtgtgaccaaatgactggcatactggcaacagatgtcagtgcggacgacgcgcaaaaactgctggtt
gggctcaaccagcgtatagtcgtcaacggtcgcacccagagaaacaccaataccatgaaaaattaccttttgcccgtagtggcccaggcattt
gctaggtgggcaaaggaatataaggaagatcaagaagatgaaaggccactaggactacgagatagacagttagtcatggggtgttgttgg
gcttttagaaggcacaagataacatctatttataagcgcccggatacccaaaccatcatcaaagtgaacagcgatttccactcattcgtgctgcc
caggataggcagtaacacattggagatcgggctgagaacaagaatcaggaaaatgttagaggagcacaaggagccgtcacctctcattacc
gccgaggacgtacaagaagctaagtgcgcagccgatgaggctaaggaggtgcgtgaagccgaggagttgcgcgcagctctaccacctttg
gcagctgatgttgaggagcccactctggaagccgatgtcgacttgatgttacaagaggctggggccggctcagtggagacacctcgtggcttg
ataaaggttaccagctacgatggcgaggacaagatcggctcttacgctgtgctttctccgcaggctgtactcaagagtgaaaaattatcttgcat
ccaccctctcgctgaacaagtcatagtgataacacactctggccgaaaagggcgttatgccgtggaaccataccatggtaaagtagtggtgcc
agagggacatgcaatacccgtccaggactttcaagctctgagtgaaagtgccaccattgtgtacaacgaacgtgagttcgtaaacaggtacct
gcaccatattgccacacatggaggagcgctgaacactgatgaagaatattacaaaactgtcaagcccagcgagcacgacggcgaatacctgt
acgacatcgacaggaaacagtgcgtcaagaaagaactagtcactgggctagggctcacaggcgagctggtggatcctcccttccatgaattc
gcctacgagagtctgagaacacgaccagccgctccttaccaagtaccaaccataggggtgtatggcgtgccaggatcaggcaagtctggcat
cattaaaagcgcagtcaccaaaaaagatctagtggtgagcgccaagaaagaaaactgtgcagaaattataagggacgtcaagaaaatgaa
agggctggacgtcaatgccagaactgtggactcagtgctcttgaatggatgcaaacaccccgtagagaccctgtatattgacgaagcttttgct
tgtcatgcaggtactctcagagcgctcatagccattataagacctaaaaaggcagtgctctgcggggatcccaaacagtgcggtttttttaacat
gatgtgcctgaaagtgcattttaaccacgagatttgcacacaagtcttccacaaaagcatctctcgccgttgcactaaatctgtgacttcggtcgt
ctcaaccttgttttacgacaaaaaaatgagaacgacgaatccgaaagagactaagattgtgattgacactaccggcagtaccaaacctaagca
ggacgatctcattctcacttgtttcagagggtgggtgaagcagttgcaaatagattacaaaggcaacgaaataatgacggcagctgcctctca
agggctgacccgtaaaggtgtgtatgccgttcggtacaaggtgaatgaaaatcctctgtacgcacccacctcagaacatgtgaacgtcctactg
acccgcacggaggaccgcatcgtgtggaaaacactagccggcgacccatggataaaaacactgactgccaagtaccctgggaatttcactgc
cacgatagaggagtggcaagcagagcatgatgccatcatgaggcacatcttggagagaccggaccctaccgacgtcttccagaataaggca
aacgtgtgttgggccaaggctttagtgccggtgctgaagaccgctggcatagacatgaccactgaacaatggaacactgtggattattttgaaa
cggacaaagctcactcagcagagatagtattgaaccaactatgcgtgaggttctttggactcgatctggactccggtctattttctgcacccactg
ttccgttatccattaggaataatcactgggataactccccgtcgcctaacatgtacgggctgaataaagaagtggtccgtcagctctctcgcagg
tacccacaactgcctcgggcagttgccactggaagagtctatgacatgaacactggtacactgcgcaattatgatccgcgcataaacctagtac
ctgtaaacagaagactgcctcatgctttagtcctccaccataatgaacacccacagagtgacttttcttcattcgtcagcaaattgaagggcaga
actgtcctggtggtcggggaaaagttgtccgtcccaggcaaaatggttgactggttgtcagaccggcctgaggctaccttcagagctcggctg
gatttaggcatcccaggtgatgtgcccaaatatgacataatatttgttaatgtgaggaccccatataaataccatcactatcagcagtgtgaaga
ccatgccattaagcttagcatgttgaccaagaaagcttgtctgcatctgaatcccggcggaacctgtgtcagcataggttatggttacgctgaca
gggccagcgaaagcatcattggtgctatagcgcggcagttcaagttttcccgggtatgcaaaccgaaatcctcacttgaagagacggaagttc
tgtttgtattcattgggtacgatcgcaaggcccgtacgcacaatccttacaagctttcatcaaccttgaccaacatttatacaggttccagactcca
cgaagccggatgtgcaccctcatatcatgtggtgcgaggggatattgccacggccaccgaaggagtgattataaatgctgctaacagcaaag
gacaacctggcggaggggtgtgcggagcgctgtataagaaattcccggaaagcttcgatttacagccgatcgaagtaggaaaagcgcgact
ggtcaaaggtgcagctaaacatatcattcatgccgtaggaccaaacttcaacaaagtttcggaggttgaaggtgacaaacagttggcagagg
cttatgagtccatcgctaagattgtcaacgataacaattacaagtcagtagcgattccactgttgtccaccggcatcttttccgggaacaaagatc
gactaacccaatcattgaaccatttgctgacagctttagacaccactgatgcagatgtagccatatactgcagggacaagaaatgggaaatga
ctctcaaggaagcagtggctaggagagaagcagtggaggagatatgcatatccgacgactcttcagtgacagaacctgatgcagagctggt
gagggtgcatccgaagagttctttggctggaaggaagggctacagcacaagcgatggcaaaactttctcatatttggaagggaccaagtttca
ccaggcggccaaggatatagcagaaattaatgccatgtggcccgttgcaacggaggccaatgagcaggtatgcatgtatatcctcggagaaa
gcatgagcagtattaggtcgaaatgccccgtcgaagagtcggaagcctccacaccacctagcacgctgccttgcttgtgcatccatgccatgac
tccagaaagagtacagcgcctaaaagcctcacgtccagaacaaattactgtgtgctcatcctttccattgccgaagtatagaatcactggtgtgc
agaagatccaatgctcccagcctatattgttctcaccgaaagtgcctgcgtatattcatccaaggaagtatctcgtggaaacaccaccggtagac
gagactccggagccatcggcagagaaccaatccacagaggggacacctgaacaaccaccacttataaccgaggatgagaccaggactaga
acgcctgagccgatcatcatcgaagaggaagaagaggatagcataagtttgctgtcagatggcccgacccaccaggtgctgcaagtcgagg
cagacattcacgggccgccctctgtatctagctcatcctggtccattcctcatgcatccgactttgatgtggacagtttatccatacttgacaccctg
gagggagctagcgtgaccagcggggcaacgtcagccgagactaactcttacttcgcaaagagtatggagtttctggcgcgaccggtgcctgc
gcctcgaacagtattcaggaaccctccacatcccgctccgcgcacaagaacaccgtcacttgcacccagcagggcctgctcgagaaccagcct
agtttccaccccgccaggcgtgaatagggtgatcactagagaggagctcgaggcgcttaccccgtcacgcactcctagcaggtcggtctcgag
aaccagcctggtctccaacccgccaggcgtaaatagggtgattacaagagaggagtttgaggcgttcgtagcacaacaacaatgacggtttg
atgcgggtgcatacatcttttcctccgacaccggtcaagggcatttacaacaaaaatcagtaaggcaaacggtgctatccgaagtggtgttgga
gaggaccgaattggagatttcgtatgccccgcgcctcgaccaagaaaaagaagaattactacgcaagaaattacagttaaatcccacacctg
ctaacagaagcagataccagtccaggaaggtggagaacatgaaagccataacagctagacgtattctgcaaggcctagggcattatttgaa
ggcagaaggaaaagtggagtgctaccgaaccctgcatcctgttcctttgtattcatctagtgtgaaccgtgccttttcaagccccaaggtcgcag
tggaagcctgtaacgccatgttgaaagagaactttccgactgtggcttcttactgtattattccagagtacgatgcctatttggacatggttgacg
gagcttcatgctgcttagacactgccagtttttgccctgcaaagctgcgcagctttccaaagaaacactcctatttggaacccacaatacgatcg
gcagtgccttcagcgatccagaacacgctccagaacgtcctggcagctgccacaaaaagaaattgcaatgtcacgcaaatgagagaattgcc
cgtattggattcggcggcctttaatgtggaatgcttcaagaaatatgcgtgtaataatgaatattgggaaacgtttaaagaaaaccccatcagg
cttactgaagaaaacgtggtaaattacattaccaaattaaaaggaccaaaagctgctgctctttttgcgaagacacataatttgaatatgttgca
ggacataccaatggacaggtttgtaatggacttaaagagagacgtgaaagtgactccaggaacaaaacatactgaagaacggcccaaggta
caggtgatccaggctgccgatccgctagcaacagcgtatctgtgcggaatccaccgagagctggttaggagattaaatgcggtcctgcttccg
aacattcatacactgtttgatatgtcggctgaagactttgacgctattatagccgagcacttccagcctggggattgtgttctggaaactgacatc
gcgtcgtttgataaaagtgaggacgacgccatggctctgaccgcgttaatgattctggaagacttaggtgtggacgcagagctgttgacgctg
attgaggcggctttcggcgaaatttcatcaatacatttgcccactaaaactaaatttaaattcggagccatgatgaaatctggaatgttcctcaca
ctgtttgtgaacacagtcattaacattgtaatcgcaagcagagtgttgagagaacggctaaccggatcaccatgtgcagcattcattggagatg
acaatatcgtgaaaggagtcaaatcggacaaattaatggcagacaggtgcgccacctggttgaatatggaagtcaagattatagatgctgtg
gtgggcgagaaagcgccttatttctgtggagggtttattttgtgtgactccgtgaccggcacagcgtgccgtgtggcagaccccctaaaaaggc
tgtttaagcttggcaaacctctggcagcagacgatgaacatgatgatgacaggagaagggcattgcatgaagagtcaacacgctggaaccg
agtgggtattctttcagagctgtgcaaggcagtagaatcaaggtatgaaaccgtaggaacttccatcatagttatggccatgactactctagcta
gcagtgttaaatcattcagctacctgagaggggcccctataactctctacggctaacctgaatggactacgacatagtctagtccgccaagggc
gcgcccacccagcggccgcatacagcagcaattggcaagctgcttacatagaactcgcggcgattggcatgccgccttaaaatttttattttattt
ttcttttcttttccgaatcggattttgtttttaatatttcaaaaaaaaaaaaaaaaaaaaaaaaaaaaaaaaaaaaaaaagaagagcgtttaaac
acgtgatatctggcctcatgggccttcctttcactgcccgctttccagtcgggaaacctgtcgtgccagctgcattaacatggtcatagctgtttcct
tgcgtattgggcgctctccgcttcctcgctcactgactcgctgcgctcggtcgttcgggtaaagcctggggtgcctaatgagcaaaaggccagc
aaaaggccaggaaccgtaaaaaggccgcgttgctggcgtttttccataggctccgcccccctgacgagcatcacaaaaatcgacgctcaagtc
agaggtggcgaaacccgacaggactataaagataccaggcgtttccccctggaagctccctcgtgcgctctcctgttccgaccctgccgcttac
cggatacctgtccgcctttctcccttcgggaagcgtggcgctttctcatagctcacgctgtaggtatctcagttcggtgtaggtcgttcgctccaag
ctgggctgtgtgcacgaaccccccgttcagcccgaccgctgcgccttatccggtaactatcgtcttgagtccaacccggtaagacacgacttatc
gccactggcagcagccactggtaacaggattagcagagcgaggtatgtaggcggtgctacagagttcttgaagtggtggcctaactacggct
acactagaagaacagtatttggtatctgcgctctgctgaagccagttaccttcggaaaaagagttggtagctcttgatccggcaaacaaaccac
cgctggtagcggtggtttttttgtttgcaagcagcagattacgcgcagaaaaaaaggatctcaagaagatcctttgatcttttctacggggtctga
cgctcagtggaacgaaaactcacgttaagggattttggtcatgaatacacggtgcctgactgcgttagcaatttaactgtgataaactaccgcat
taaagcttatcgatgataagctgtcaaacatgagaattcttagaaaaactcatcgagcatcaaatgaaactgcaatttattcatatcaggattatc
aataccatatttttgaaaaagccgtttctgtaatgaaggagaaaactcaccgaggcagttccataggatggcaagatcctggtatcggtctgcg
attccgactcgtccaacatcaatacaacctattaatttcccctcgtcaaaaataaggttatcaagtgagaaatcaccatgagtgacgactgaatc
cggtgagaatggcaaaagcttatgcatttctttccagacttgttcaacaggccagccattacgctcgtcatcaaaatcactcgcatcaaccaaac
cgttattcattcgtgattgcgcctgagcgagacgaaatacgcgatcgctgttaaaaggacaattacaaacaggaatcgaatgcaaccggcgca
ggaacactgccagcgcatcaacaatattttcacctgaatcaggatattcttctaatacctggaatgctgttttcccggggatcgcagtggtgagta
accatgcatcatcaggagtacggataaaatgcttgatggtcggaagaggcataaattccgtcagccagtttagtctgaccatctcatctgtaaca
tcattggcaacgctacctttgccatgtttcagaaacaactctggcgcatcgggcttcccatacaatcgatagattgtcgcacctgattgcccgaca
ttatcgcgagcccatttatacccatataaatcagcatccatgttggaatttaatcgcggcctcgagcaagacgtttcccgttgaatatggctcataa
caccccttgtattactgtttatgtaagcagacagttttattgttcatgagcggatacatatttgaatgtatttagaaaaataaacaaataggggttc
cgcgcacatttccccgaaaagtgccacctaaattgtaagcgttaatattttgttaaaattcgcgttaaatttttgttaaatcagctcattttttaacca
ataggccgaaatcggcaaaatcccttataaatcaaaagaatagaccgagatagggttgagtggccgctacagggcgctcccattcgccattca
ggctgcgcaactgttgggaagggcgtttcggtgcgggcctcttcgctattacgccagctggcgaaagggggatgtgctgcaaggcgattaagt
tgggtaacgccagggttttcccagtcacacgcgtaatacgactcactatag
SEQ ID NO: 3: nucleotide cDNA sequence of hemagglutinin (HA) from influenza virus
A/California/07/2009 (H1N1)
atgaaggcaatactagtagttctgctatatacatttgcaaccgcaaatgcagacacattatgtataggttatcatgcgaacaattcaacagacac
tgtagacacagtactagaaaagaatgtaacagtaacacactctgttaaccttctagaagacaagcataacgggaaactatgcaaactaagag
gggtagccccattgcatttgggtaaatgtaacattgctggctggatcctgggaaatccagagtgtgaatcactctccacagcaagctcatggtcc
tacattgtggaaacacctagttcagacaatggaacgtgttacccaggagatttcatcgattatgaggagctaagagagcaattgagctcagtgt
catcatttgaaaggtttgagatattccccaagacaagttcatggcccaatcatgactcgaacaaaggtgtaacggcagcatgtcctcatgctgg
agcaaaaagcttctacaaaaatttaatatggctagttaaaaaaggaaattcatacccaaagctcagcaaatcctacattaatgataaagggaa
agaagtcctcgtgctatggggcattcaccatccatctactagtgctgaccaacaaagtctctatcagaatgcagatgcatatgtttttgtggggtc
atcaagatacagcaagacgttcaagccggaaatagcaataagacccaaagtgagggatcgagaagggagaatgaactattactggacact
agtagagccgggagacaaaataacattcgaagcaactggaaatctagtggtaccgagatatgcattcgcaatggaaagaaatgctggatct
ggtattatcatttcagatacaccagtccacgattgcaatacaacttgtcaaacacccaagggtgctataaacaccagcctcccatttcagaatata
catccgatcacaattggaaaatgtccaaaatatgtaaaaagcacaaaattgagactggccacaggattgaggaatatcccgtctattcaatcta
gaggcctatttggggccattgccggtttcattgaaggggggtggacagggatggtagatggatggtacggttatcaccatcaaaatgagcag
gggtcaggatatgcagccgacctgaagagcacacagaatgccattgacgagattactaacaaagtaaattctgttattgaaaagatgaatac
acagttcacagcagtaggtaaagagttcaaccacctggaaaaaagaatagagaatttaaataaaaaagttgatgatggtttcctggacatttg
gacttacaatgccgaactgttggttctattggaaaatgaaagaactttggactaccacgattcaaatgtgaagaacttatatgaaaaggtaaga
agccagctaaaaaacaatgccaaggaaattggaaacggctgctttgaattttaccacaaatgcgataacacgtgcatggaaagtgtcaaaaa
tgggacttatgactacccaaaatactcagaggaagcaaaattaaacagagaagaaatagatggggtaaagctggaatcaacaaggatttac
cagattttggcgatctattcaactgtcgccagttcattggtactggtagtctccctgggggcaatcagtttctggatgtgctctaatgggtctctaca
gtgtagaatatgtatttgataa
SEQ ID NO: 4: amino acid sequence of HA from influenza virus A/California/07/2009 (H1N1)
MKAILVVLLYTFATANADTLCIGYHANNSTDTVDTVLEKNVTVTHSVNLLEDKHNGKLCKLRGVAPLHLGKCNIA
GWILGNPECESLSTASSWSYIVETPSSDNGTCYPGDFIDYEELREQLSSVSSFERFEIFPKTSSWPNHDSNKGVT
AACPHAGAKSFYKNLIWLVKKGNSYPKLSKSYINDKGKEVLVLWGIHHPSTSADQQSLYQNADAYVFVGSSRYS
KTFKPEIAIRPKVRDREGRMNYYWTLVEPGDKITFEATGNLVVPRYAFAMERNAGSGIIISDTPVHDCNTTCQTP
KGAINTSLPFQNIHPITIGKCPKYVKSTKLRLATGLRNIPSIQSRGLFGAIAGFIEGGWTGMVDGWYGYHHQNEQ
GSGYAADLKSTQNAIDEITNKVNSVIEKMNTQFTAVGKEFNHLEKRIENLNKKVDDGFLDIWTYNAELLVLLENE
RTLDYHDSNVKNLYEKVRSQLKNNAKEIGNGCFEFYHKCDNTCMESVKNGTYDYPKYSEEAKLNREEIDGVKLE
STRIYQILAIYSTVASSLVLVVSLGAISFWMCSNGSLQCRICI
SEQ ID NO: 5: nucleotide DNA sequence of HA from influenza virus A/PR/8/34 (H1N1)
ATGAAGGCAAACCTACTGGTCCTGTTATGTGCACTTGCAGCTGCAGATGCAGACACAATATGTATAGGCTAC
CATACGAACAATTCAACCGACACTGTTGACACAGTACTCGAGAAGAATGTGACAGTGACACACTCTGTTAACC
TGCTCGAAGACAGCCACAACGGAAAACTATGTAGATTAAAAGGAATAGCCCCACTACAATTGGGGAAATGTA
ACATCGCCGGATGGCTCTTGGGAAACCCAGAATGCGACCCACTGCTTCCAGTGAGATCATGGTCCTACATTG
TAGAAACACCAAACTCTGAGAATGGAATATGTTATCCAGGAGATTTCATCGACTATGAGGAGCTGAGGGAGC
AATTGAGCTCAGTGTCATCATTCGAAAGATTCGAAATATTTCCCAAAGAAAGCTCATGGCCCAACCACAACAC
AAACGGAGTAACGGCAGCATGCTCCCATGAGGGGAAAAGCAGTTTTTACAGAAATTTGCTATGGCTGACGGA
GAAGGAGGGCTCATACCCAAAGCTGAAAAATTCTTATGTGAACAAAAAAGGGAAAGAAGTCCTTGTACTGTG
GGGTATTCATCACCCGCCTAACAGTAAGGAACAACAGAATCTCTATCAGAATGAAAATGCTTATGTCTCTGTA
GTGACTTCAAATTATAACAGGAGATTTACCCCGGAAATAGCAGAAAGACCCAAAGTAAGAGATCAAGCTGGG
AGGATGAACTATTACTGGACCTTGCTAAAACCCGGAGACACAATAATATTTGAGGCAAATGGAAATCTAATAG
CACCAATGTATGCTTTCGCACTGAGTAGAGGCTTTGGGTCCGGCATCATCACCTCAAACGCATCAATGCATG
AGTGTAACACGAAGTGTCAAACACCCCTGGGAGCTATAAACAGCAGTCTCCCTTACCAGAATATACACCCAGT
CACAATAGGAGAGTGCCCAAAATACGTCAGGAGTGCCAAATTGAGGATGGTTACAGGACTAAGGAACATTCC
GTCCATTCAATCCAGAGGTCTATTTGGAGCCATTGCCGGTTTTATTGAAGGGGGATGGACTGGAATGATAGA
TGGATGGTATGGTTATCATCATCAGAATGAACAGGGATCAGGCTATGCAGCGGATCAAAAAAGCACACAAAA
TGCCATTAACGGGATTACAAACAAGGTGAACACTGTTATCGAGAAAATGAACATTCAATTCACAGCTGTGGG
TAAAGAATTCAACAAATTAGAAAAAAGGATGGAAAATTTAAATAAAAAAGTTGATGATGGATTTCTGGACATT
TGGACATATAATGCAGAATTGTTAGTTCTACTGGAAAATGAAAGGACTCTGGAATTCCATGACTCAAATGTGA
AGAATCTGTATGAGAAAGTAAAAAGCCAATTAAAGAATAATGCCAAAGAAATCGGAAATGGATGTTTTGAGT
TCTACCACAAGTGTGACAATGAATGCATGGAAAGTGTAAGAAATGGGACTTATGATTATCCCAAATATTCAGA
AGAGTCAAAGTTGAACAGGGAAAAGGTAGATGGAGTGAAATTGGAATCAATGGGGATCTATCAGATTCTGGC
GATCTACTCAACTGTCGCCAGTTCACTGGTGCTTTTGGTCTCCCTGGGGGCAATCAGTTTCTGGATGTGTTC
TAATGGATCTTTGCAGTGCAGAATATGCATCTGATAA
SEQ ID NO: 6: amino acid sequence of HA from influenza virus A/PR/8/34 (H1N1)
MKANLLVLLCALAAADADTICIGYHTNNSTDTVDTVLEKNVTVTHSVNLLEDSHNGKLCRLKGIAPLQLGKCNIA
GWLLGNPECDPLLPVRSWSYIVETPNSENGICYPGDFIDYEELREQLSSVSSFERFEIFPKESSWPNHNTNGVTA
ACSHEGKSSFYRNLLWLTEKEGSYPKLKNSYVNKKGKEVLVLWGIHHPPNSKEQQNLYQNENAYVSVVTSNYNR
RFTPEIAERPKVRDQAGRMNYYWTLLKPGDTIIFEANGNLIAPMYAFALSRGFGSGIITSNASMHECNTKCQTPL
GAINSSLPYQNIHPVTIGECPKYVRSAKLRMVTGLRNIPSIQSRGLFGAIAGFIEGGWTGMIDGWYGYHHQNEQ
GSGYAADQKSTQNAINGITNKVNTVIEKMNIQFTAVGKEFNKLEKRMENLNKKVDDGFLDIWTYNAELLVLLEN
ERTLEFHDSNVKNLYEKVKSQLKNNAKEIGNGCFEFYHKCDNECMESVRNGTYDYPKYSEESKLNREKVDGVKL
ESMGIYQILAIYSTVASSLVLLVSLGAISFWMCSNGSLQCRICI
SEQ ID NO: 7: nucleotide cDNA sequence of HA from influenza virus A/Bilthoven/16398/1968 (H3N2)
GGGCCCCTATAACTCTCTACGGCTAACCTGAATGGACTACGACATAGTCTAGTCCGCCAAGATGAAGACCAT
CATTGCCCTGAGCTACATCTTCTGCCTGGCCCTGGGACAGGACCTGCCCGGCAACGACAATAGCACCGCCAC
CCTGTGTCTGGGCCACCACGCCGTGCCTAACGGCACCCTGGTGAAAACCATCACCGACGACCAGATCGAAGT
GACCAACGCCACCGAGCTGGTGCAGAGCAGCAGCACCGGCAAGATCTGCAACAACCCCCACCGGATCCTGGA
CGGCATCAACTGCACCCTGATCGACGCCCTGCTGGGCGACCCCCACTGCGACGTGTTCCAGGACGAGACATG
GGACCTGTTCGTGGAAAGAAGCAAGGCCTTCAGCAACTGCTACCCCTACGACGTGCCCGACTACGCCAGCCT
GAGAAGCCTGGTGGCCAGCAGCGGCACACTGGAATTCATCACCGAGGGCTTCACCTGGACCGGCGTGACCC
AGAACGGCGGCAGCAACGCCTGCAAGAGAGGCCCTGGCAGCGGCTTCTTCAGCAGACTGAACTGGCTGACC
AAGAGCGGCAGCACCTACCCCGTGCTGAACGTGACCATGCCCAACAACGACAACTTCGACAAGCTGTACATC
TGGGGCGTGCACCACCCCAGCACCAACCAGGAACAGACCAGCCTGTACGTGCAGGCCAGCGGCAGAGTGAC
CGTGTCCACCAGAAGAAGCCAGCAGACCATCATCCCCAACATCGGCAGCAGACCCTGGGTCCGCGGCCTGAG
CAGCCGGATCAGCATCTACTGGACCATCGTGAAGCCCGGCGACGTGCTGGTGATCAACAGCAACGGCAACCT
GATCGCCCCCAGAGGCTACTTCAAGATGCGGACCGGCAAGAGCAGCATCATGCGGAGCGACGCCCCCATCGA
TACCTGCATCAGCGAGTGCATCACCCCCAACGGCAGCATCCCCAACGACAAGCCCTTCCAGAACGTGAACAA
GATCACCTACGGCGCCTGCCCCAAATACGTGAAGCAGAACACCCTGAAGCTGGCCACCGGCATGCGGAACGT
GCCCGAGAAGCAGACCAGAGGCCTGTTCGGCGCCATTGCCGGCTTCATCGAGAACGGCTGGGAGGGCATGA
TCGACGGTTGGTACGGCTTCCGGCACCAGAACAGCGAGGGCACAGGACAGGCCGCCGACCTGAAGTCTACC
CAGGCCGCCATCGACCAGATCAACGGCAAGCTGAACAGAGTGATCGAAAAGACCAACGAGAAGTTCCACCAG
ATCGAGAAAGAATTCAGCGAGGTGGAAGGCCGGATCCAGGACCTGGAAAAGTACGTGGAAGATACCAAGAT
CGACCTGTGGTCCTACAACGCCGAGCTGCTGGTGGCCCTGGAAAACCAGCACACCATCGACCTGACCGACAG
CGAGATGAACAAGCTGTTCGAGAAAACCAGACGGCAGCTGCGCGAGAACGCCGAGGACATGGGCAACGGCT
GCTTCAAGATCTACCACAAGTGCGACAATGCCTGCATCGAGAGCATCCGGAACGGCACCTACGACCACGACG
TGTACAGGGACGAGGCCCTGAACAACCGGTTCCAGATCAAGGGCGTGGAACTGAAGTCCGGCTACAAGGAC
TGGATCCTGTGGATCAGCTTCGCCATCAGCTGCTTTCTGCTGTGCGTGGTGCTGCTGGGCTTCATCATGTGG
GCCTGCCAGCGGGGCAACATCCGGTGCAACATCTGCATTTAAGGCGCGCCCACCCAGCGGCCGC
SEQ ID NO: 8: amino acid sequence of HA from influenza virus A/Bilthoven/16398/1968 (H3N2)
MKTIIALSYIFCLALGQDLPGNDNSTATLCLGHHAVPNGTLVKTITDDQIEVTNATELVQSSSTGKICNNPHRILD
GINCTLIDALLGDPHCDVFQDETWDLFVERSKAFSNCYPYDVPDYASLRSLVASSGTLEFITEGFTWTGVTQNG
GSNACKRGPGSGFFSRLNWLTKSGSTYPVLNVTMPNNDNFDKLYIWGVHHPSTNQEQTSLYVQASGRVTVSTR
RSQQTIIPNIGSRPWVRGLSSRISIYWTIVKPGDVLVINSNGNLIAPRGYFKMRTGKSSIMRSDAPIDTCISECITP
NGSIPNDKPFQNVNKITYGACPKYVKQNTLKLATGMRNVPEKQTRGLFGAIAGFIENGWEGMIDGWYGFRHQN
SEGTGQAADLKSTQAAIDQINGKLNRVIEKTNEKFHQIEKEFSEVEGRIQDLEKYVEDTKIDLWSYNAELLVALEN
QHTIDLTDSEMNKLFEKTRRQLRENAEDMGNGCFKIYHKCDNACIESIRNGTYDHDVYRDEALNNRFQIKGVEL
KSGYKDWILWISFAISCFLLCVVLLGFIMWACQRGNIRCNICI
SEQ ID NO: 9: nucleotide cDNA sequence of HA from influenza virus A/Bangkok/1/79 (H3N2)
GGGCCCCTATAACTCTCTACGGCTAACCTGAATGGACTACGACATAGTCTAGTCCGCCAAGATGAAGACCAT
CATTGCCCTGAGCTACATCTTCTGCCTGGTGTTCGCCCAGAACCTGCCCGGCAACGACAATAGCACCGCCAC
CCTGTGTCTGGGCCACCACGCCGTGCCTAACGGCACCCTGGTGAAAACCATCACCAACGACCAGATCGAAGT
GACCAACGCCACCGAGCTGGTGCAGAGCAGCAGCACCGGCAGAATCTGCGACAGCCCCCACCGGATCCTGGA
CGGCAAGAACTGCACCCTGATCGACGCCCTGCTGGGCGACCCCCACTGCGACGGCTTCCAGAACGAGAAGTG
GGACCTGTTCGTGGAAAGAAGCAAGGCCTTCAGCAACTGCTACCCCTACGACGTGCCCGACTACGCCAGCCT
GAGAAGCCTGGTGGCCAGCAGCGGCACACTGGAATTCATCAACGAGGGCTTCAACTGGACCGGCGTGACCC
AGAGCGGCGGCAGCTATGCCTGCAAGAGAGGCAGCGACAAGAGCTTCTTCAGCAGACTGAACTGGCTGTAC
GAGAGCGAGAGCAAGTACCCCGTGCTGAACGTGACCATGCCCAACAACGGCAACTTCGACAAGCTGTACATC
TGGGGCGTGCACCACCCCAGCACCGACAAAGAACAGACCAACCTGTACGTGCGGGCCAGCGGCAGAGTGAC
CGTGTCCACCAAGAGAAGCCAGCAGACCATCATCCCCAACATCGGCAGCAGACCCTGGGTCCGCGGCCTGAG
CAGCGGCATCAGCATCTACTGGACCATCGTGAAGCCCGGCGACATCCTGCTGATCAACAGCAACGGCAACCT
GATCGCCCCCAGAGGCTACTTCAAGATCCGGACCGGCAAGAGCAGCATCATGCGGAGCGACGCCCCCATCGG
GACCTGCAGCAGCGAGTGCATCACCCCCAACGGCAGCATCCCCAACGACAAGCCCTTCCAGAACGTGAACAA
GATCACCTACGGCGCCTGCCCCAAATACGTGAAGCAGAACACCCTGAAGCTGGCCACCGGCATGCGGAACGT
GCCCGAGAAGCAGACCAGAGGCATCTTCGGCGCCATTGCCGGCTTCATCGAGAACGGCTGGGAGGGCATGG
TGGACGGTTGGTACGGCTTCCGGCACCAGAACAGCGAGGGCACAGGACAGGCCGCCGACCTGAAGTCTACC
CAGGCCGCCATCGACCAGATCAACGGCAAGCTGAACAGAGTGATCGAAAAGACCAACGAGAAGTTCCACCAG
ATCGAGAAAGAATTCAGCGAGGTGGAAGGCCGGATCCAGGACCTGGAAAAGTACGTGGAAGATACCAAGAT
CGACCTGTGGTCCTACAACGCCGAGCTGCTGGTGGCCCTGGAAAACCAGCACACCATCGACCTGACCGACAG
CGAGATGAACAAGCTGTTCGAGAAAACCAGACGGCAGCTGCGCGAGAACGCCGAGGACATGGGCAACGGCT
GCTTCAAGATCTACCACAAGTGCGACAATGCCTGCATCGGCAGCATCCGGAACGGCACCTACGACCACGACG
TGTACAGGGACGAGGCCCTGAACAACCGGTTCCAGATCAAGGGCGTGGAACTGAAGTCCGGCTACAAGGAC
TGGATCCTGTGGATCAGCTTCGCCATCAGCTGCTTTCTGCTGTGCGTGGTGCTGCTGGGCTTCATCATGTGG
GCCTGCCAGAAAGGCAACATCCGGTGCAACATCTGCATTTAAGGCGCGCCCACCCAGCGGCCGC
SEQ ID NO: 10: amino acid sequence of HA from influenza virus A/Bangkok/1/79 (H3N2)
MKTIIALSYIFCLVFAQNLPGNDNSTATLCLGHHAVPNGTLVKTITNDQIEVTNATELVQSSSTGRICDSPHRILD
GKNCTLIDALLGDPHCDGFQNEKWDLFVERSKAFSNCYPYDVPDYASLRSLVASSGTLEFINEGFNWTGVTQSG
GSYACKRGSDKSFFSRLNWLYESESKYPVLNVTMPNNGNFDKLYIWGVHHPSTDKEQTNLYVRASGRVTVSTK
RSQQTIIPNIGSRPWVRGLSSGISIYWTIVKPGDILLINSNGNLIAPRGYFKIRTGKSSIMRSDAPIGTCSSECITPN
GSIPNDKPFQNVNKITYGACPKYVKQNTLKLATGMRNVPEKQTRGIFGAIAGFIENGWEGMVDGWYGFRHQNS
EGTGQAADLKSTQAAIDQINGKLNRVIEKTNEKFHQIEKEFSEVEGRIQDLEKYVEDTKIDLWSYNAELLVALEN
QHTIDLTDSEMNKLFEKTRRQLRENAEDMGNGCFKIYHKCDNACIGSIRNGTYDHDVYRDEALNNRFQIKGVEL
KSGYKDWILWISFAISCFLLCVVLLGFIMWACQKGNIRCNICI
SEQ ID NO: 11: nucleotide cDNA sequence of HA from influenza virus A/Beijing/32/92 (H3N2)
GGGCCCCTATAACTCTCTACGGCTAACCTGAATGGACTACGACATAGTCTAGTCCGCCAAGATGAAGACCAT
CATTGCCCTGAGCTACATCCTGTGCCTGGTGTTCGCCCAGAAGCTGCCCGGCAACGACAATAGCACCGCCAC
CCTGTGTCTGGGCCACCACGCCGTGCCTAACGGCACCCTGGTGAAAACCATCACCAACGACCAGATCGAAGT
GACCAACGCCACCGAGCTGGTGCAGAGCAGCAGCACCGGCAGAATCTGCGACAGCCCCCACCGGATCCTGGA
CGGCAAGAACTGCACCCTGATCGACGCCCTGCTGGGCGACCCCCACTGCGACGGCTTCCAGAACAAAGAGTG
GGACCTGTTCGTGGAAAGAAGCAAGGCCTACAGCAACTGCTACCCCTACGACGTGCCCGACTACGCCAGCCT
GAGAAGCCTGGTGGCCAGCAGCGGCACACTGGAATTCATCAACGAGGACTTCAACTGGACCGGCGTGGCCC
AGGATGGCGGCAGCTATGCCTGCAAGAGAGGCAGCGTGAACAGCTTCTTCAGCAGACTGAACTGGCTGCACA
AGAGCGAGTACAAGTACCCCGCCCTGAACGTGACCATGCCCAACAACGGCAAGTTCGACAAGCTGTACATCT
GGGGCGTGCACCACCCCAGCACCGACAGGGACCAGACCAGCCTGTACGTGCGGGCCAGCGGCAGAGTGACC
GTGTCCACCAAGAGAAGCCAGCAGACCGTGACCCCCAACATCGGCAGCAGACCCTGGGTCCGCGGCCAGAGC
AGCCGGATCAGCATCTACTGGACCATCGTGAAGCCCGGCGACATCCTGCTGATCAACAGCACAGGCAACCTG
ATCGCCCCCAGAGGCTACTTCAAGATCCGGAACGGCAAGAGCAGCATCATGCGGAGCGACGCCCCCATCGGG
ACCTGCAGCAGCGAGTGCATCACCCCCAACGGCAGCATCCCCAACGACAAGCCCTTCCAGAACGTGAACCGG
ATCACCTACGGCGCCTGCCCCAGATACGTGAAGCAGAACACCCTGAAGCTGGCCACCGGCATGCGGAACGTG
CCCGAGAAGCAGACCAGAGGCATCTTCGGCGCCATTGCCGGCTTCATCGAGAACGGCTGGGAGGGCATGGT
GGACGGTTGGTACGGCTTCCGGCACCAGAACAGCGAGGGCACAGGACAGGCCGCCGACCTGAAGTCTACCC
AGGCCGCCATCGACCAGATCAACGGCAAGCTGAACAGACTGATCGAAAAGACCAACGAGAAGTTCCACCAGA
TCGAGAAAGAATTCAGCGAGGTGGAAGGCCGGATCCAGGACCTGGAAAAGTACGTGGAAGATACCAAGATC
GACCTGTGGTCCTACAACGCCGAGCTGCTGGTGGCCCTGGAAAACCAGCACACCATCGACCTGACCGACAGC
GAGATGAACAAGCTGTTCGAGAAAACCAGAAAGCAGCTGCGCGAGAACGCCGAGGACATGGGCAACGGCTG
CTTCAAGATCTACCACAAGTGCGACAATGCCTGCATCGGCAGCATCCGGAACGGCACCTACGACCACGACGT
GTACAGGGACGAGGCCCTGAACAACCGGTTCCAGATCAAGGGCGTGGAACTGAAGTCCGGCTACAAGGACT
GGATCCTGTGGATCAGCTTCGCCATCAGCTGCTTTCTGCTGTGCGTGGTGCTGCTGGGCTTCATCATGTGGG
CCTGCCAGAAAGGCAACATCCGGTGCAACATCTGCATTTAAGGCGCGCCCACCCAGCGGCCGC
SEQ ID NO: 12: amino acid sequence of HA from influenza virus A/Beijing/32/92 (H3N2)
MKTIIALSYILCLVFAQKLPGNDNSTATLCLGHHAVPNGTLVKTITNDQIEVTNATELVQSSSTGRICDSPHRILD
GKNCTLIDALLGDPHCDGFQNKEWDLFVERSKAYSNCYPYDVPDYASLRSLVASSGTLEFINEDFNWTGVAQDG
GSYACKRGSVNSFFSRLNWLHKSEYKYPALNVTMPNNGKFDKLYIWGVHHPSTDRDQTSLYVRASGRVTVSTK
RSQQTVTPNIGSRPWVRGQSSRISIYWTIVKPGDILLINSTGNLIAPRGYFKIRNGKSSIMRSDAPIGTCSSECITP
NGSIPNDKPFQNVNRITYGACPRYVKQNTLKLATGMRNVPEKQTRGIFGAIAGFIENGWEGMVDGWYGFRHQN
SEGTGQAADLKSTQAAIDQINGKLNRLIEKTNEKFHQIEKEFSEVEGRIQDLEKYVEDTKIDLWSYNAELLVALEN
QHTIDLTDSEMNKLFEKTRKQLRENAEDMGNGCFKIYHKCDNACIGSIRNGTYDHDVYRDEALNNRFQIKGVEL
KSGYKDWILWISFAISCFLLCVVLLGFIMWACQKGNIRCNICI
SEQ ID NO: 13: nucleotide cDNA sequence of HA from influenza virus A/Fujain/411/2002 (H3N2)
GGGCCCCTATAACTCTCTACGGCTAACCTGAATGGACTACGACATAGTCTAGTCCGCCAAGATGAAGACCAT
CATTGCCCTGAGCTACATCCTGTGCCTGGTGTTCGCCCAGAAGCTGCCCGGCAACGACAATAGCACCGCCAC
CCTGTGTCTGGGCCACCACGCCGTGCCTAACGGCACCATCGTGAAAACCATCACCAACGACCAGATCGAAGT
GACCAACGCCACCGAGCTGGTGCAGAGCAGCAGCACCGGCGGCATCTGCGACAGCCCCCACCAGATCCTGGA
CGGCGAGAACTGCACCCTGATCGACGCCCTGCTGGGCGACCCCCAGTGCGACGGCTTCCAGAACAAGAAATG
GGACCTGTTCGTGGAAAGAAGCAAGGCCTACAGCAACTGCTACCCCTACGACGTGCCCGACTACGCCAGCCT
GAGAAGCCTGGTGGCCAGCAGCGGCACACTGGAATTCAACAACGAGAGCTTCAACTGGACCGGCGTGACCCA
GAACGGCACCAGCAGCGCCTGCAAGAGAAGAAGCAACAAGAGCTTCTTCAGCAGACTGAACTGGCTGACCCA
CCTGAAGTACAAGTACCCCGCCCTGAACGTGACCATGCCCAACAACGAAAAGTTCGACAAGCTGTACATCTG
GGGCGTGCTGCACCCCGGCACCGACAGCGACCAGATCAGCCTGTACGCCCAGGCCAGCGGCAGAATCACCG
TGTCCACCAAGAGAAGCCAGCAGACCGTGATCCCCAACATCGGCAGCAGACCCAGAGTCCGCGGCGTGAGCA
GCCGGATCAGCATCTACTGGACCATCGTGAAGCCCGGCGACATCCTGCTGATCAACAGCACCGGCAACCTGA
TCGCCCCCAGAGGCTACTTCAAGATTCGGAGCGGCAAGAGCAGCATCATGCGGAGCGACGCCCCCATCGGCA
AGTGCAACAGCGAGTGCATCACCCCCAACGGCAGCATCCCCAACGACAAGCCCTTCCAGAACGTGAACCGGA
TCACCTACGGCGCCTGCCCCCGGTACATCAAGCAGAACACCCTGAAGCTGGCCACCGGCATGCGGAACGTGC
CCGAGAAGCAGACCAGAGGCATCTTCGGCGCCATTGCCGGCTTCATCGAGAACGGCTGGGAGGGCATGGTG
GACGGTTGGTACGGCTTCCGGCACCAGAACAGCGAGGGCACAGGACAGGCCGCCGACCTGAAGTCTACCCA
GGCCGCCATCAACCAGATCAACGGCAAGCTGAACAGACTGATCGGCAAGACCAACGAGAAGTTCCACCAGAT
CGAGAAAGAATTCAGCGAGGTGGAAGGCCGGATCCAGGACCTGGAAAAGTACGTGGAAGATACCAAGATCG
ACCTGTGGTCCTACAACGCCGAGCTGCTGGTGGCCCTGGAAAACCAGCACACCATCGACCTGACCGACAGCG
AGATGAACAAGCTGTTCGAGCGGACCAAGAAGCAGCTGCGCGAGAACGCCGAGGACATGGGCAACGGCTGC
TTCAAGATCTACCACAAGTGCGACAATGCCTGCATCGGCAGCATCCGGAACGGCACCTACGACCACGACGTG
TACAGGGACGAGGCCCTGAACAACCGGTTCCAGATCAAGGGCGTGGAACTGAAGTCCGGCTACAAGGACTG
GATCCTGTGGATCAGCTTCGCCATCAGCTGCTTTCTGCTGTGCGTGGCCCTGCTGGGCTTCATCATGTGGGC
CTGCCAGAAAGGCAACATCCGGTGCAACATCTGCATTTAAGGCGCGCCCACCCAGCGGCCGC
SEQ ID NO: 14: amino acid sequence of HA from influenza virus A/Fujain/411/2002 (H3N2)
MKTIIALSYILCLVFAQKLPGNDNSTATLCLGHHAVPNGTIVKTITNDQIEVTNATELVQSSSTGGICDSPHQILD
GENCTLIDALLGDPQCDGFQNKKWDLFVERSKAYSNCYPYDVPDYASLRSLVASSGTLEFNNESFNWTGVTQN
GTSSACKRRSNKSFFSRLNWLTHLKYKYPALNVTMPNNEKFDKLYIWGVLHPGTDSDQISLYAQASGRITVSTK
RSQQTVIPNIGSRPRVRGVSSRISIYWTIVKPGDILLINSTGNLIAPRGYFKIRSGKSSIMRSDAPIGKCNSECITPN
GSIPNDKPFQNVNRITYGACPRYIKQNTLKLATGMRNVPEKQTRGIFGAIAGFIENGWEGMVDGWYGFRHQNS
EGTGQAADLKSTQAAINQINGKLNRLIGKTNEKFHQIEKEFSEVEGRIQDLEKYVEDTKIDLWSYNAELLVALEN
QHTIDLTDSEMNKLFERTKKQLRENAEDMGNGCFKIYHKCDNACIGSIRNGTYDHDVYRDEALNNRFQIKGVEL
KSGYKDWILWISFAISCFLLCVALLGFIMWACQKGNIRCNICI
SEQ ID NO: 15: nucleotide cDNA sequence of HA from influenza virus A/Brisbane/10/2007 (H3N2)
GGGCCCCTATAACTCTCTACGGCTAACCTGAATGGACTACGACATAGTCTAGTCCGCCAAGATGAAGACCAT
CATTGCCCTGAGCTACATCCTGTGCCTGGTGTTCACCCAGAAGCTGCCCGGCAACGACAATAGCACCGCCAC
CCTGTGTCTGGGCCACCACGCCGTGCCTAACGGCACCATCGTGAAAACCATCACCAACGACCAGATCGAAGT
GACCAACGCCACCGAGCTGGTGCAGAGCAGCAGCACCGGCGAGATCTGCGACAGCCCCCACCAGATCCTGGA
CGGCGAGAACTGCACCCTGATCGACGCCCTGCTGGGCGACCCCCAGTGCGACGGCTTCCAGAACAAGAAATG
GGACCTGTTCGTGGAAAGAAGCAAGGCCTACAGCAACTGCTACCCCTACGACGTGCCCGACTACGCCAGCCT
GAGAAGCCTGGTGGCCAGCAGCGGCACACTGGAATTCAACAACGAGAGCTTCAACTGGACCGGCGTGACCCA
GAACGGCACCAGCAGCGCCTGCATCAGAAGAAGCAACAACAGCTTCTTCAGCAGACTGAACTGGCTGACCCA
CCTGAAGTTCAAGTACCCCGCCCTGAACGTGACCATGCCCAACAACGAAAAGTTCGACAAGCTGTACATCTG
GGGCGTGCACCACCCCGGCACCGACAACGATCAGATCTTCCCATACGCCCAGGCCAGCGGCAGAATCACCGT
GTCCACCAAGAGAAGCCAGCAGACCGTGATCCCCAACATCGGCAGCAGACCCAGAGTCCGCAACATCCCCAG
CCGGATCAGCATCTACTGGACCATCGTGAAGCCCGGCGACATCCTGCTGATCAACAGCACCGGCAACCTGAT
CGCCCCCAGAGGCTACTTCAAGATTCGGAGCGGCAAGAGCAGCATCATGCGGAGCGACGCCCCCATCGGCAA
GTGCAACAGCGAGTGCATCACCCCCAACGGCAGCATCCCCAACGACAAGCCCTTCCAGAACGTGAACCGGAT
CACCTACGGCGCCTGCCCCAGATACGTGAAGCAGAACACCCTGAAGCTGGCCACCGGCATGCGGAACGTGCC
CGAGAAGCAGACCAGAGGCATCTTCGGCGCCATTGCCGGCTTCATCGAGAACGGCTGGGAGGGCATGGTGG
ACGGTTGGTACGGCTTCCGGCACCAGAACAGCGAGGGCATTGGACAGGCCGCCGACCTGAAGTCTACCCAG
GCCGCCATCGACCAGATCAACGGCAAGCTGAACAGACTGATCGGCAAGACCAACGAGAAGTTCCACCAGATC
GAGAAAGAATTCAGCGAGGTGGAAGGCCGGATCCAGGACCTGGAAAAGTACGTGGAAGATACCAAGATCGA
CCTGTGGTCCTACAACGCCGAGCTGCTGGTGGCCCTGGAAAACCAGCACACCATCGACCTGACCGACAGCGA
GATGAACAAGCTGTTCGAGAAAACCAAGAAGCAGCTGCGCGAGAACGCCGAGGACATGGGCAACGGCTGCT
TCAAGATCTACCACAAGTGCGACAATGCCTGCATCGGCAGCATCCGGAACGGCACCTACGACCACAACGTGT
ACAGGGACGAGGCCCTGAACAACCGGTTCCAGATCAAGGGCGTGGAACTGAAGTCCGGCTACAAGGACTGG
ATCCTGTGGATCAGCTTCGCCATCAGCTGCTTTCTGCTGTGCGTGGCCCTGCTGGGCTTCATCATGTGGGCC
TGCCAGAAAGGCAACATCCGGTGCAACATCTGCATTTAAGGCGCGCCCACCCAGCGGCCGC
SEQ ID NO: 16: amino acid sequence of HA from influenza virus A/Brisbane/10/2007 (H3N2)
MKTIIALSYILCLVFTQKLPGNDNSTATLCLGHHAVPNGTIVKTITNDQIEVTNATELVQSSSTGEICDSPHQILDG
ENCTLIDALLGDPQCDGFQNKKWDLFVERSKAYSNCYPYDVPDYASLRSLVASSGTLEFNNESFNWTGVTQNGT
SSACIRRSNNSFFSRLNWLTHLKFKYPALNVTMPNNEKFDKLYIWGVHHPGTDNDQIFPYAQASGRITVSTKRS
QQTVIPNIGSRPRVRNIPSRISIYWTIVKPGDILLINSTGNLIAPRGYFKIRSGKSSIMRSDAPIGKCNSECITPNGS
IPNDKPFQNVNRITYGACPRYVKQNTLKLATGMRNVPEKQTRGIFGAIAGFIENGWEGMVDGWYGFRHQNSEG
IGQAADLKSTQAAIDQINGKLNRLIGKTNEKFHQIEKEFSEVEGRIQDLEKYVEDTKIDLWSYNAELLVALENQHT
IDLTDSEMNKLFEKTKKQLRENAEDMGNGCFKIYHKCDNACIGSIRNGTYDHNVYRDEALNNRFQIKGVELKSG
YKDWILWISFAISCFLLCVALLGFIMWACQKGNIRCNICI
SEQ ID NO: 17: nucleotide cDNA sequence of HA from influenza virus A/Texas/50/2012 (H3N2)
GGGCCCCTATAACTCTCTACGGCTAACCTGAATGGACTACGACATAGTCTAGTCCGCCAAGATGAAGACCAT
CATTGCCCTGAGCTACATCCTGTGCCTGGTGTTCGCCCAGAAGCTGCCCGGCAACGACAATAGCACCGCCAC
CCTGTGTCTGGGCCACCACGCCGTGCCTAACGGCACCATCGTGAAAACCATCACCAACGACCGGATCGAAGT
GACCAACGCCACCGAGCTGGTGCAGAACAGCAGCATCGGCGAGATCTGCGACAGCCCCCACCAGATCCTGGA
CGGCGAGAACTGCACCCTGATCGACGCCCTGCTGGGCGACCCCCAGTGCGACGGCTTCCAGAACAAGAAATG
GGACCTGTTCGTGGAAAGAAGCAAGGCCTACAGCAACTGCTACCCCTACGACGTGCCCGACTACGCCAGCCT
GAGAAGCCTGGTGGCCAGCAGCGGCACACTGGAATTCAACAACGAGAGCTTCAACTGGAACGGCGTGACCCA
GAACGGCACCAGCAGCGCCTGCATCAGAAGAAGCAACAACAGCTTCTTCAGCAGACTGAACTGGCTGACCCA
CCTGAACTTCAAGTACCCCGCCCTGAACGTGACCATGCCCAACAACGAGCAGTTCGACAAGCTGTACATCTG
GGGCGTGCACCACCCCGGCACCGACAAGGACCAGATCTTCCTGTACGCCCAGCCCAGCGGCAGAATCACCGT
GTCCACCAAGAGAAGCCAGCAGGCCGTGATCCCCAACATCGGCAGCAGACCCCGGATCCGCAACATCCCCAG
CCGGATCAGCATCTACTGGACCATCGTGAAGCCCGGCGACATCCTGCTGATCAACAGCACCGGCAACCTGAT
CGCCCCCAGAGGCTACTTCAAGATTCGGAGCGGCAAGAGCAGCATCATGCGGAGCGACGCCCCCATCGGCAA
GTGCAAGAGCGAGTGCATCACCCCCAACGGCAGCATCCCCAACGACAAGCCCTTCCAGAACGTGAACCGGAT
CACCTACGGCGCCTGCCCCAGATACGTGAAGCAGAGCACCCTGAAGCTGGCCACCGGCATGCGGAACGTGCC
CGAGAAGCAGACCAGAGGCATCTTCGGCGCCATTGCCGGCTTCATCGAGAACGGCTGGGAGGGCATGGTGG
ACGGTTGGTACGGCTTCCGGCACCAGAACAGCGAGGGCAGAGGACAGGCCGCCGACCTGAAGTCTACCCAG
GCCGCCATCGACCAGATCAACGGCAAGCTGAACAGACTGATCGGCAAGACCAACGAGAAGTTCCACCAGATC
GAGAAAGAATTCAGCGAGGTGGAAGGCCGGATCCAGGACCTGGAAAAGTACGTGGAAGATACCAAGATCGA
CCTGTGGTCCTACAACGCCGAGCTGCTGGTGGCCCTGGAAAACCAGCACACCATCGACCTGACCGACAGCGA
GATGAACAAGCTGTTCGAGAAAACCAAGAAGCAGCTGCGCGAGAACGCCGAGGACATGGGCAACGGCTGCT
TCAAGATCTACCACAAGTGCGACAATGCCTGCATCGGCAGCATCCGGAACGGCACCTACGACCACGACGTGT
ACAGGGACGAGGCCCTGAACAACCGGTTCCAGATCAAGGGCGTGGAACTGAAGTCCGGCTACAAGGACTGG
ATCCTGTGGATCAGCTTCGCCATCAGCTGCTTTCTGCTGTGCGTGGCCCTGCTGGGCTTCATCATGTGGGCC
TGCCAGAAAGGCAACATCCGGTGCAACATCTGCATTTAAGGCGCGCCCACCCAGCGGCCGC
SEQ ID NO: 18: amino acid sequence of HA from influenza virus A/Texas/50/2012
(H3N2)MKTIIALSYILCLVFAQKLPGNDNSTATLCLGHHAVPNGTIVKTITNDRIEVTNATELVQNSSIGEICDSP
HQILDGENCTLIDALLGDPQCDGFQNKKWDLFVERSKAYSNCYPYDVPDYASLRSLVASSGTLEFNNESFNWNG
VTQNGTSSACIRRSNNSFFSRLNWLTHLNFKYPALNVTMPNNEQFDKLYIWGVHHPGTDKDQIFLYAQPSGRIT
VSTKRSQQAVIPNIGSRPRIRNIPSRISIYWTIVKPGDILLINSTGNLIAPRGYFKIRSGKSSIMRSDAPIGKCKSECI
TPNGSIPNDKPFQNVNRITYGACPRYVKQSTLKLATGMRNVPEKQTRGIFGAIAGFIENGWEGMVDGWYGFRH
QNSEGRGQAADLKSTQAAIDQINGKLNRLIGKTNEKFHQIEKEFSEVEGRIQDLEKYVEDTKIDLWSYNAELLVA
LENQHTIDLTDSEMNKLFEKTKKQLRENAEDMGNGCFKIYHKCDNACIGSIRNGTYDHDVYRDEALNNRFQIKG
VELKSGYKDWILWISFAISCFLLCVALLGFIMWACQKGNIRCNICI
SEQ ID NO: 19: nucleotide cDNA sequence of HA from influenza virus A/turkey/Turkey/1/2005
(H5N1)
atggagaaaatagtgcttcttcttgcaatagtcagccttgttaaaagtgatcagatttgcattggttaccatgcaaacaactcgacagagcaggtt
gacacaataatggaaaagaacgtcactgttacacacgcccaagacatactggaaaagacacacaacgggaaactctgcgatctagatggag
tgaagcctctaattttaagagattgtagtgtagctggatggctcctcgggaacccaatgtgtgacgaattcctcaatgtgccggaatggtcttaca
tagtggagaagatcaatccagccaatgacctctgttacccagggaatttcaacgactatgaagaactgaaacacctattgagcagaataaacc
attttgagaaaattcagatcatccccaaaagttcttggtcagatcatgaagcctcagcaggggtgagctcagcatgtccataccagggaaggtc
ctccttttttagaaatgtggtatggcttatcaaaaaggacaatgcatacccaacaataaagagaagttacaataataccaaccaagaagatcttt
tggtattgtgggggattcaccatccaaatgatgcggcagagcagacaaggctctatcaaaacccaactacctatatttccgttgggacatcaac
actaaaccagagattggtaccaaaaatagccactagatctaaggtaaacgggcaaagtggaaggatggagttcttttggacaattttaaaacc
gaatgatgcaataaactttgagagtaatggaaatttcattgctccagaaaatgcatacaaaattgtcaagaaaggggactcaacaattatgaa
aagtgagttggaatatggtaactgcaacaccaagtgtcaaactccaataggggcgataaactctagtatgccattccacaacatccaccctctc
accatcggggaatgccccaaatatgtgaaatcaagcagattagtccttgctactgggctcagaaatagccctcaacgagagacacgaggact
atttggagctatagcaggttttatagagggaggatggcagggaatggtagatggttggtatgggtaccaccatagcaacgagcaggggagt
gggtacgctgcagacaaagaatccactcaaaaggcaatagatggagtcaccaataaggtcaactcgatcattgacaaaatgaacactcagtt
tgaggctgttggaagggaatttaataacttagaaaggagaatagaaaatttaaacaagaagatggaagacggattcctagatgtctggactt
ataatgctgaacttctggttctcatggaaaatgagagaactctagactttcatgactcaaatgtcaagaacctttacgacaaggtccgactacag
cttagggataatgcaaaggagcttggtaacggttgtttcgagttctatcacagatgtgataatgaatgtatggaaagtgtaagaaacggaacgt
atgactacccgcagtattcagaagaagcaagattaaaaagagaggaaataagtggagtaaaattggaatcaataggaacttaccaaatact
gtcaatttattcaacagtggcgagctccctagcactggcaatcatggtggctggtctatctttatggatgtgctccaatggatcgttacaatgcag
aatttgcatttaa
SEQ ID NO: 20: amino acid sequence of HA from influenza virus A/turkey/Turkey/1/2005 (H5N1)
MEKIVLLLAIVSLVKSDQICIGYHANNSTEQVDTIMEKNVTVTHAQDILEKTHNGKLCDLDGVKPLILRDCSVAG
WLLGNPMCDEFLNVPEWSYIVEKINPANDLCYPGNFNDYEELKHLLSRINHFEKIQIIPKSSWSDHEASAGVSSA
CPYQGRSSFFRNVVWLIKKDNAYPTIKRSYNNTNQEDLLVLWGIHHPNDAAEQTRLYQNPTTYISVGTSTLNQR
LVPKIATRSKVNGQSGRMEFFWTILKPNDAINFESNGNFIAPENAYKIVKKGDSTIMKSELEYGNCNTKCQTPIGA
INSSMPFHNIHPLTIGECPKYVKSSRLVLATGLRNSPQRETRGLFGAIAGFIEGGWQGMVDGWYGYHHSNEQGS
GYAADKESTQKAIDGVTNKVNSIIDKMNTQFEAVGREFNNLERRIENLNKKMEDGFLDVWTYNAELLVLMENER
TLDFHDSNVKNLYDKVRLQLRDNAKELGNGCFEFYHRCDNECMESVRNGTYDYPQYSEEARLKREEISGVKLESI
GTYQILSIYSTVASSLALAIMVAGLSLWMCSNGSLQCRICI
SEQ ID NO: 21: nucleotide cDNA sequence of HA from influenza virus A/Shanghai/2/2013 (H7N9)
ATGAACACTCAAATCCTGGTATTCGCTCTGATTGCGATCATTCCAACAAATGCAGACAAAATCTGCCTCGGAC
ATCATGCCGTGTCAAACGGAACCAAAGTAAACACATTAACTGAAAGAGGAGTGGAAGTCGTCAATGCAACTG
AAACAGTGGAACGAACAAACATCCCCAGGATCTGCTCAAAAGGGAAAAGGACAGTTGACCTCGGTCAATGTG
GACTCCTGGGGACAATCACTGGACCACCTCAATGTGACCAATTCCTAGAATTTTCAGCCGATTTAATTATTGA
GAGGCGAGAAGGAAGTGATGTCTGTTATCCTGGGAAATTCGTGAATGAAGAAGCTCTGAGGCAAATTCTCAG
AGAATCAGGCGGAATTGACAAGGAAGCAATGGGATTCACATACAGTGGAATAAGAACTAATGGAGCAACCAG
TGCATGTAGGAGATCAGGATCTTCATTCTATGCAGAAATGAAATGGCTCCTGTCAAACACAGATAATGCTGC
ATTCCCGCAGATGACTAAGTCATATAAAAATACAAGAAAAAGCCCAGCTCTAATAGTATGGGGGATCCATCAT
TCCGTATCAACTGCAGAGCAAACCAAGCTATATGGGAGTGGAAACAAACTGGTGACAGTTGGGAGTTCTAAT
TATCAACAATCTTTTGTACCGAGTCCAGGAGCGAGACCACAAGTTAATGGTCTATCTGGAAGAATTGACTTTC
ATTGGCTAATGCTAAATCCCAATGATACAGTCACTTTCAGTTTCAATGGGGCTTTCATAGCTCCAGACCGTGC
AAGCTTCCTGAGAGGAAAATCTATGGGAATCCAGAGTGGAGTACAGGTTGATGCCAATTGTGAAGGGGACTG
CTATCATAGTGGAGGGACAATAATAAGTAACTTGCCATTTCAGAACATAGATAGCAGGGCAGTTGGAAAATG
TCCGAGATATGTTAAGCAAAGGAGTCTGCTGCTAGCAACAGGGATGAAGAATGTTCCTGAGATTCCAAAAGG
AAGAGGCCTATTTGGTGCTATAGCGGGTTTCATTGAAAATGGATGGGAAGGCCTAATTGATGGTTGGTATGG
TTTCAGACACCAGAATGCACAGGGAGAGGGAACTGCTGCAGATTACAAAAGCACTCAATCGGCAATTGATCA
AATAACAGGAAAATTAAACCGGCTTATAGAAAAAACCAACCAACAATTTGAGTTGATAGACAATGAATTCAAT
GAGGTAGAGAAGCAAATCGGTAATGTGATAAATTGGACCAGAGATTCTATAACAGAAGTGTGGTCATACAAT
GCTGAACTCTTGGTAGCAATGGAGAACCAGCATACAATTGATCTGGCTGATTCAGAAATGGACAAACTGTAC
GAACGAGTGAAAAGACAGCTGAGAGAGAATGCTGAAGAAGATGGCACTGGTTGCTTTGAAATATTTCACAAG
TGTGATGATGACTGTATGGCCAGTATTAGAAATAACACCTATGATCACAGCAAATACAGGGAAGAGGCAA
SEQ ID NO: 22: amino acid sequence of HA from influenza virus A/Shanghai/2/2013 (H7N9)
MNTQILVFALIAIIPTNADKICLGHHAVSNGTKVNTLTERGVEVVNATETVERTNIPRICSKGKRTVDLGQCGLLG
TITGPPQCDQFLEFSADLIIERREGSDVCYPGKFVNEEALRQILRESGGIDKEAMGFTYSGIRTNGATSACRRSGS
SFYAEMKWLLSNTDNAAFPQMTKSYKNTRKSPALIVWGIHHSVSTAEQTKLYGSGNKLVTVGSSNYQQSFVPSP
GARPQVNGLSGRIDFHWLMLNPNDTVTFSFNGAFIAPDRASFLRGKSMGIQSGVQVDANCEGDCYHSGGTIISN
LPFQNIDSRAVGKCPRYVKQRSLLLATGMKNVPEIPKGRGLFGAIAGFIENGWEGLIDGWYGFRHQNAQGEGTA
ADYKSTQSAIDQITGKLNRLIEKTNQQFELIDNEFNEVEKQIGNVINWTRDSITEVWSYNAELLVAMENQHTIDL
ADSEMDKLYERVKRQLRENAEEDGTGCFEIFHKCDDDCMASIRNNTYDHSKYREEAX
SEQ ID NO: 23: forward primer for influenza H1N1 A/California/07/2009 insert
ATTCCCGTCGACGCCACCATGAAGGCAATACTAGTAGTTCT
SEQ ID NO: 24 reverse primer for influenza H1N1 A/California/07/2009 insert
ATTTACGCCTAGGTTATCAAATACATATTCTACACTGTAGAGAC
SEQ ID NO: 25: forward primer for H7 HA gene from A/Shanghai/2/2013
AATTAAGTCGACGCCACCATGAACACTCAAATCCTGGTATTCG
SEQ ID NO: 26: reverse primer for H7 HA gene from A/Shanghai/2/2013
AATTAATCTAGATTATCATATACAAATAGTGCACCGCATG
SEQ ID NO: 27 peptide
ALNNRFQIKGVELKS
SEQ ID NO: 28 SB63 primer
catagtctagtcgacgccaccatggagaaaatagtgcttcttcttgc
SEQ ID NO: 29 SB74 primer
gtcgaagttcagggtctgcttcacgggggccacgatcttctgcttgtgccgggcctcccgcttggcccgaatgcaaattctgcattgtaacgatc
SEQ ID NO: 30 SB76 primer
gtgaagcagaccctgaacttcgacctgctgaagctggccggcgacgtggagagcaaccccggccccatgaaggcaatactagtagttctgc
SEQ ID NO: 31 SB66 primer
ggcgtagcggcggccgcttatcaaatacatattctacactgtagagaccca

1-29. (canceled)

30. An immunogenic composition comprising:
(i) a first self-replicating RNA molecule encoding a polypeptide comprising a first antigen; and
(ii) a second self-replicating RNA molecule encoding a polypeptide comprising a second antigen,
wherein the first antigen and the second antigen are both HA from the same subtype of influenza virus, but the first antigen is from a different strain of influenza virus to the second antigen as follows:
(a) the first antigen is HA from a pandemic influenza A subtype or an immunogenic fragment or variant thereof, and the second antigen is HA from a different pandemic influenza A strain to the first antigen or an immunogenic fragment or variant thereof, or
(b) the first antigen is HA from a seasonal influenza A subtype or an immunogenic fragment or variant thereof, and the second antigen is HA from a different seasonal influenza A strain to the first antigen or an immunogenic fragment or variant thereof.

31. The immunogenic composition of claim 30, wherein the first antigen and the second antigen are the only antigens from influenza virus in the self-replicating RNA molecules.

32. The immunogenic composition of claim 30, further comprising: (iii) a third self-replicating RNA molecule encoding a polypeptide comprising a third antigen, wherein the third antigen is from influenza virus, but is from a different strain of influenza virus to both the first and the second antigen.

33. The immunogenic composition of claim 32, wherein the first, second, and third antigens are the only antigens derived from influenza virus in the self-replicating RNA molecules.

34. The immunogenic composition of claim 32, further comprising: (iv) a fourth self-replicating RNA molecule encoding a polypeptide comprising a fourth antigen, wherein the fourth antigen is from influenza virus, but is from a different strain of influenza virus to the first antigen, the second antigen, and the third antigen.

35. The immunogenic composition of claim 30, wherein the first antigen is HA from pandemic influenza A subtype H5 or an immunogenic fragment or variant thereof, and the second antigen is HA from a different pandemic influenza A subtype H5 strain to the first antigen or an immunogenic fragment or variant thereof.

36. The immunogenic composition of claim 30, wherein the first antigen is HA from seasonal influenza A subtype H1 or an immunogenic fragment or variant thereof, and the second antigen is HA from a different seasonal influenza A subtype H1 strain to the first antigen or an immunogenic fragment or variant thereof.

37. The immunogenic composition of claim 30, wherein the first antigen is HA from seasonal influenza A subtype H3 or an immunogenic fragment or variant thereof, and the second antigen is HA from a different seasonal influenza A subtype H3 strain to the first antigen or an immunogenic fragment or variant thereof.

38. The immunogenic composition of claim 30, further comprising an adjuvant.

39. The immunogenic composition of claim 30, wherein the self-replicating RNA molecule is derived from an alphavirus.

40. The immunogenic composition of claim 39, wherein the alphavirus is selected from the group consisting of: Sindbis (SIN), Venezuelan equine encephalitis (VEE), Semliki Forest virus (SFV), or a combination thereof.

41. A pharmaceutical composition comprising the immunogenic composition of claim 30 and a pharmaceutically acceptable carrier.

42. The pharmaceutical composition of claim 41, further comprising a cationic lipid, a liposome, a microparticle, viral replicon particles (VRPs), an oil-in-water emulsion, or a cationic nanoemulsion.

43. The pharmaceutical composition of claim 42, wherein the self-replicating RNA molecules are encapsulated in, bound to, or adsorbed on a cationic lipid, a liposome, a microparticle, viral replicon particles (VRPs), an oil-in-water emulsion or a cationic nanoemulsion.

44. A method of prevention and/or treatment against influenza disease, comprising administering an effective amount of the immunogenic composition of claim 30 to a person in need thereof.

45. A method of inducing an immune response in a person, comprising administering to the person an effective amount of the pharmaceutical composition of claim 41.

46. A method of prevention and/or treatment against influenza disease, comprising the steps of:
(i) administering an effective amount of a first immunogenic composition to a person in need thereof, wherein the first immunogenic composition comprises a first self-replicating RNA molecule encoding a polypeptide comprising a first antigen and pharmaceutically acceptable carrier; and
(ii) simultaneous, at substantially the same time, or sequential administration of a second immunogenic composition comprising a second self-replicating RNA molecule encoding a polypeptide comprising a second antigen and pharmaceutically acceptable carrier,
wherein the first and second antigens are both HA from the same subtype of influenza virus, but the first antigen is from a different strain of influenza to the second antigen as follows:
(a) the first antigen is HA from a pandemic influenza A subtype or an immunogenic fragment or variant thereof, and the second antigen is HA from a different pandemic influenza A strain to the first antigen or an immunogenic fragment or variant thereof; or
(b) the first antigen is HA from a seasonal influenza A subtype or an immunogenic fragment or variant thereof, and the second antigen is HA from a different seasonal influenza A strain to the first antigen or an immunogenic fragment or variant thereof.

47. The method of claim 46, wherein the first antigen is HA from pandemic influenza A subtype H5 or an immunogenic fragment or variant thereof, and the second antigen is HA from a different pandemic influenza A subtype H5 strain to the first antigen or an immunogenic fragment or variant thereof.

48. The method of claim 46, wherein the first antigen is HA from seasonal influenza A subtype H3 or an immunogenic fragment or variant thereof, and the second antigen is HA from a different seasonal influenza A subtype H3 strain to the first antigen or an immunogenic fragment or variant thereof.


SEQUENCE LISTING (31 sequences)

SEQ ID NO: 1 - DNA, 11688 bp, synthetic construct
  misc_feature 1..11688: note = Nucleotide cDNA sequence of A836 TC83 VEE/SINV (Venezuelan equine encephalitis-Sindbis virus) chimeric replicon containing T7 DNA polymerase promoter
  source 1..11688: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggcagggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaaggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 2 - DNA, 9993 bp, synthetic construct
  misc_feature 1..9993: note = Nucleotide cDNA sequence of A836 TC83 VEE/SINV chimeric replicon without insert
  source 1..9993: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 3 - DNA, 1704 bp, Influenza virus
  source 1..1704: mol_type = unassigned DNA
atgaaggcaatactagtagttctgctatatacatttgcaaccgcaaatgcagacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacacctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgcagatgcatatgtttttgtggggtcatcaagatacagcaagacgttcaagccggaaatagcaataagacccaaagtgagggatcgagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcaaacacccaagggtgctataaacaccagcctcccatttcagaatatacatccgatcacaattggaaaatgtccaaaatatgtaaaaagcacaaaattgagactggccacaggattgaggaatatcccgtctattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagctaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatttgataa

SEQ ID NO: 4 - protein, 566 aa, Influenza virus
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKTFKPEIAIRPKVRDREGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 5 - DNA, 1701 bp, Influenza virus
  source 1..1701: mol_type = unassigned DNA
atgaaggcaaacctactggtcctgttatgtgcacttgcagctgcagatgcagacacaatatgtataggctaccatacgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatctctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacattccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggaattccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctgataa

SEQ ID NO: 6 - protein, 565 aa, Influenza virus
MKANLLVLLCALAAADADTICIGYHTNNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLEFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 7 - DNA, 1784 bp, Influenza virus
  source 1..1784: mol_type = unassigned DNA
gggcccctataactctctacggctaacctgaatggactacgacatagtctagtccgccaagatgaagaccatcattgccctgagctacatcttctgcctggccctgggacaggacctgcccggcaacgacaatagcaccgccaccctgtgtctgggccaccacgccgtgcctaacggcaccctggtgaaaaccatcaccgacgaccagatcgaagtgaccaacgccaccgagctggtgcagagcagcagcaccggcaagatctgcaacaacccccaccggatcctggacggcatcaactgcaccctgatcgacgccctgctgggcgacccccactgcgacgtgttccaggacgagacatgggacctgttcgtggaaagaagcaaggccttcagcaactgctacccctacgacgtgcccgactacgccagcctgagaagcctggtggccagcagcggcacactggaattcatcaccgagggcttcacctggaccggcgtgacccagaacggcggcagcaacgcctgcaagagaggccctggcagcggcttcttcagcagactgaactggctgaccaagagcggcagcacctaccccgtgctgaacgtgaccatgcccaacaacgacaacttcgacaagctgtacatctggggcgtgcaccaccccagcaccaaccaggaacagaccagcctgtacgtgcaggccagcggcagagtgaccgtgtccaccagaagaagccagcagaccatcatccccaacatcggcagcagaccctgggtccgcggcctgagcagccggatcagcatctactggaccatcgtgaagcccggcgacgtgctggtgatcaacagcaacggcaacctgatcgcccccagaggctacttcaagatgcggaccggcaagagcagcatcatgcggagcgacgcccccatcgatacctgcatcagcgagtgcatcacccccaacggcagcatccccaacgacaagcccttccagaacgtgaacaagatcacctacggcgcctgccccaaatacgtgaagcagaacaccctgaagctggccaccggcatgcggaacgtgcccgagaagcagaccagaggcctgttcggcgccattgccggcttcatcgagaacggctgggagggcatgatcgacggttggtacggcttccggcaccagaacagcgagggcacaggacaggccgccgacctgaagtctacccaggccgccatcgaccagatcaacggcaagctgaacagagtgatcgaaaagaccaacgagaagttccaccagatcgagaaagaattcagcgaggtggaaggccggatccaggacctggaaaagtacgtggaagataccaagatcgacctgtggtcctacaacgccgagctgctggtggccctggaaaaccagcacaccatcgacctgaccgacagcgagatgaacaagctgttcgagaaaaccagacggcagctgcgcgagaacgccgaggacatgggcaacggctgcttcaagatctaccacaagtgcgacaatgcctgcatcgagagcatccggaacggcacctacgaccacgacgtgtacagggacgaggccctgaacaaccggttccagatcaagggcgtggaactgaagtccggctacaaggactggatcctgtggatcagcttcgccatcagctgctttctgctgtgcgtggtgctgctgggcttcatcatgtgggcctgccagcggggcaacatccggtgcaacatctgcatttaaggcgcgcccacccagcggccgc

SEQ ID NO: 8 - protein, 566 aa, Influenza virus
MKTIIALSYIFCLALGQDLPGNDNSTATLCLGHHAVPNGTLVKTITDDQIEVTNATELVQSSSTGKICNNPHRILDGINCTLIDALLGDPHCDVFQDETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGVHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIESIRNGTYDHDVYRDEALNNRFQIKGVELKSGYKDWILWISFAISCFLLCVVLLGFIMWACQRGNIRCNICI

SEQ ID NO: 9 - DNA, 1784 bp, Influenza virus
  source 1..1784: mol_type = unassigned DNA
gggcccctataactctctacggctaacctgaatggactacgacatagtctagtccgccaagatgaagaccatcattgccctgagctacatcttctgcctggtgttcgcccagaacctgcccggcaacgacaatagcaccgccaccctgtgtctgggccaccacgccgtgcctaacggcaccctggtgaaaaccatcaccaacgaccagatcgaagtgaccaacgccaccgagctggtgcagagcagcagcaccggcagaatctgcgacagcccccaccggatcctggacggcaagaactgcaccctgatcgacgccctgctgggcgacccccactgcgacggcttccagaacgagaagtgggacctgttcgtggaaagaagcaaggccttcagcaactgctacccctacgacgtgcccgactacgccagcctgagaagcctggtggccagcagcggcacactggaattcatcaacgagggcttcaactggaccggcgtgacccagagcggcggcagctatgcctgcaagagaggcagcgacaagagcttcttcagcagactgaactggctgtacgagagcgagagcaagtaccccgtgctgaacgtgaccatgcccaacaacggcaacttcgacaagctgtacatctggggcgtgcaccaccccagcaccgacaaagaacagaccaacctgtacgtgcgggccagcggcagagtgaccgtgtccaccaagagaagccagcagaccatcatccccaacatcggcagcagaccctgggtccgcggcctgagcagcggcatcagcatctactggaccatcgtgaagcccggcgacatcctgctgatcaacagcaacggcaacctgatcgcccccagaggctacttcaagatccggaccggcaagagcagcatcatgcggagcgacgcccccatcgggacctgcagcagcgagtgcatcacccccaacggcagcatccccaacgacaagcccttccagaacgtgaacaagatcacctacggcgcctgccccaaatacgtgaagcagaacaccctgaagctggccaccggcatgcggaacgtgcccgagaagcagaccagaggcatcttcggcgccattgccggcttcatcgagaacggctgggagggcatggtggacggttggtacggcttccggcaccagaacagcgagggcacaggacaggccgccgacctgaagtctacccaggccgccatcgaccagatcaacggcaagctgaacagagtgatcgaaaagaccaacgagaagttccaccagatcgagaaagaattcagcgaggtggaaggccggatccaggacctggaaaagtacgtggaagataccaagatcgacctgtggtcctacaacgccgagctgctggtggccctggaaaaccagcacaccatcgacctgaccgacagcgagatgaacaagctgttcgagaaaaccagacggcagctgcgcgagaacgccgaggacatgggcaacggctgcttcaagatctaccacaagtgcgacaatgcctgcatcggcagcatccggaacggcacctacgaccacgacgtgtacagggacgaggccctgaacaaccggttccagatcaagggcgtggaactgaagtccggctacaaggactggatcctgtggatcagcttcgccatcagctgctttctgctgtgcgtggtgctgctgggcttcatcatgtgggcctgccagaaaggcaacatccggtgcaacatctgcatttaaggcgcgcccacccagcggccgc

SEQ ID NO: 10 - protein, 566 aa, Influenza virus
MKTIIALSYIFCLVFAQNLPGNDNSTATLCLGHHAVPNGTLVKTITNDQIEVTNATELVQSSSTGRICDSPHRILDGKNCTLIDALLGDPHCDGFQNEKWDLFVERSKAFSNCYPYDVPDYASLRSLVASSGTLEFINEGFNWTGVTQSGGSYACKRGSDKSFFSRLNWLYESESKYPVLNVTMPNNGNFDKLYIWGVHHPSTDKEQTNLYVRASGRVTVSTKRSQQTIIPNIGSRPWVRGLSSGISIYWTIVKPGDILLINSNGNLIAPRGYFKIRTGKSSIMRSDAPIGTCSSECITPNGSIPNDKPFQNVNKITYGACPKYVKQNTLKLATGMRNVPEKQTRGIFGAIAGFIENGWEGMVDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 11 - DNA, 1784 bp, Influenza virus
  source 1..1784: mol_type = unassigned DNA
gggcccctataactctctacggctaacctgaatggactacgacatagtctagtccgccaagatgaagaccatcattgccctgagctacatcctgtgcctggtgttcgcccagaagctgcccggcaacgacaatagcaccgccaccctgtgtctgggccaccacgccgtgcctaacggcaccctggtgaaaaccatcaccaacgaccagatcgaagtgaccaacgccaccgagctggtgcagagcagcagcaccggcagaatctgcgacagcccccaccggatcctggacggcaagaactgcaccctgatcgacgccctgctgggcgacccccactgcgacggcttccagaacaaagagtgggacctgttcgtggaaagaagcaaggcctacagcaactgctacccctacgacgtgcccgactacgccagcctgagaagcctggtggccagcagcggcacactggaattcatcaacgaggacttcaactggaccggcgtggcccaggatggcggcagctatgcctgcaagagaggcagcgtgaacagcttcttcagcagactgaactggctgcacaagagcgagtacaagtaccccgccctgaacgtgaccatgcccaacaacggcaagttcgacaagctgtacatctggggcgtgcaccaccccagcaccgacagggaccagaccagcctgtacgtgcgggccagcggcagagtgaccgtgtccaccaagagaagccagcagaccgtgacccccaacatcggcagcagaccctgggtccgcggccagagcagccggatcagcatctactggaccatcgtgaagcccggcgacatcctgctgatcaacagcacaggcaacctgatcgcccccagaggctacttcaagatccggaacggcaagagcagcatcatgcggagcgacgcccccatcgggacctgcagcagcgagtgcatcacccccaacggcagcatccccaacgacaagcccttccagaacgtgaaccggatcacctacggcgcctgccccagatacgtgaagcagaacaccctgaagctggccaccggcatgcggaacgtgcccgagaagcagaccagaggcatcttcggcgccattgccggcttcatcgagaacggctgggagggcatggtggacggttggtacggcttccggcaccagaacagcgagggcacaggacaggccgccgacctgaagtctacccaggccgccatcgaccagatcaacggcaagctgaacagactgatcgaaaagaccaacgagaagttccaccagatcgagaaagaattcagcgaggtggaaggccggatccaggacctggaaaagtacgtggaagataccaagatcgacctgtggtcctacaacgccgagctgctggtggccctggaaaaccagcacaccatcgacctgaccgacagcgagatgaacaagctgttcgagaaaaccagaaagcagctgcgcgagaacgccgaggacatgggcaacggctgcttcaagatctaccacaagtgcgacaatgcctgcatcggcagcatccggaacggcacctacgaccacgacgtgtacagggacgaggccctgaacaaccggttccagatcaagggcgtggaactgaagtccggctacaaggactggatcctgtggatcagcttcgccatcagctgctttctgctgtgcgtggtgctgctgggcttcatcatgtgggcctgccagaaaggcaacatccggtgcaacatctgcatttaaggcgcgcccacccagcggccgc

SEQ ID NO: 12 - protein, 566 aa, Influenza virus
MKTIIALSYILCLVFAQKLPGNDNSTATLCLGHHAVPNGTLVKTITNDQIEVTNATELVQSSSTGRICDSPHRILDGKNCTLIDALLGDPHCDGFQNKEWDLFVERSKAYSNCYPYDVPDYASLRSLVASSGTLEFINEDFNWTGVAQDGGSYACKRGSVNSFFSRLNWLHKSEYKYPALNVTMPNNGKFDKLYIWGVHHPSTDRDQTSLYVRASGRVTVSTKRSQQTVTPNIGSRPWVRGQSSRISIYWTIVKPGDILLINSTGNLIAPRGYFKIRNGKSSIMRSDAPIGTCSSECITPNGSIPNDKPFQNVNRITYGACPRYVKQNTLKLATGMRNVPEKQTRGIFGAIAGFIENGWEGMVDGWYGFRHQNSEGTGQAADLKSTQAAIDQINGKLNRLIEKTNEKFHQIEKEFSEVEGRIQDLEKYVEDTKIDLWSYNAELLVALENQHTIDLTDSEMNKLFEKTRKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 13 - DNA, 1784 bp, Influenza virus
  source 1..1784: mol_type = unassigned DNA
gggcccctataactctctacggctaacctgaatggactacgacatagtctagtccgccaagatgaagaccatcattgccctgagctacatcctgtgcctggtgttcgcccagaagctgcccggcaacgacaatagcaccgccaccctgtgtctgggccaccacgccgtgcctaacggcaccatcgtgaaaaccatcaccaacgaccagatcgaagtgaccaacgccaccgagctggtgcagagcagcagcaccggcggcatctgcgacagcccccaccagatcctggacggcgagaactgcaccctgatcgacgccctgctgggcgacccccagtgcgacggcttccagaacaagaaatgggacctgttcgtggaaagaagcaaggcctacagcaactgctacccctacgacgtgcccgactacgccagcctgagaagcctggtggccagcagcggcacactggaattcaacaacgagagcttcaactggaccggcgtgacccagaacggcaccagcagcgcctgcaagagaagaagcaacaagagcttcttcagcagactgaactggctgacccacctgaagtacaagtaccccgccctgaacgtgaccatgcccaacaacgaaaagttcgacaagctgtacatctggggcgtgctgcaccccggcaccgacagcgaccagatcagcctgtacgcccaggccagcggcagaatcaccgtgtccaccaagagaagccagcagaccgtgatccccaacatcggcagcagacccagagtccgcggcgtgagcagccggatcagcatctactggaccatcgtgaagcccggcgacatcctgctgatcaacagcaccggcaacctgatcgcccccagaggctacttcaagattcggagcggcaagagcagcatcatgcggagcgacgcccccatcggcaagtgcaacagcgagtgcatcacccccaacggcagcatccccaacgacaagcccttccagaacgtgaaccggatcacctacggcgcctgcccccggtacatcaagcagaacaccctgaagctggccaccggcatgcggaacgtgcccgagaagcagaccagaggcatcttcggcgccattgccggcttcatcgagaacggctgggagggcatggtggacggttggtacggcttccggcaccagaacagcgagggcacaggacaggccgccgacctgaagtctacccaggccgccatcaaccagatcaacggcaagctgaacagactgatcggcaagaccaacgagaagttccaccagatcgagaaagaattcagcgaggtggaaggccggatccaggacctggaaaagtacgtggaagataccaagatcgacctgtggtcctacaacgccgagctgctggtggccctggaaaaccagcacaccatcgacctgaccgacagcgagatgaacaagctgttcgagcggaccaagaagcagctgcgcgagaacgccgaggacatgggcaacggctgcttcaagatctaccacaagtgcgacaatgcctgcatcggcagcatccggaacggcacctacgaccacgacgtgtacagggacgaggccctgaacaaccggttccagatcaagggcgtggaactgaagtccggctacaaggactggatcctgtggatcagcttcgccatcagctgctttctgctgtgcgtggccctgctgggcttcatcatgtgggcctgccagaaaggcaacatccggtgcaacatctgcatttaaggcgcgcccacccagcggccgc

SEQ ID NO: 14 - protein, 566 aa, Influenza virus
MKTIIALSYILCLVFAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSACKRRSNKSFFSRLNWLTHLKYKYPALNVTMPNNEKFDKLYIWGVLHPGTDSDQISLYAQASGRITVSTKRSQQTVIPNIGSRPRVRGVSSRISIYWTIVKPGDILLINSTGNLIAPRGYFKIRSGKSSIMRSDAPIGKCNSECITPNGSIPNDKPFQNVNRITYGACPRYIKQNTLKLATGMRNVPEKQTRGIFGAIAGFIENGWEGMVDGWYGFRHQNSEGTGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 15 - DNA, 1784 bp, Influenza virus
  source 1..1784: mol_type = unassigned DNA
gggcccctataactctctacggctaacctgaatggactacgacatagtctagtccgccaagatgaagaccatcattgccctgagctacatcctgtgcctggtgttcacccagaagctgcccggcaacgacaatagcaccgccaccctgtgtctgggccaccacgccgtgcctaacggcaccatcgtgaaaaccatcaccaacgaccagatcgaagtgaccaacgccaccgagctggtgcagagcagcagcaccggcgagatctgcgacagcccccaccagatcctggacggcgagaactgcaccctgatcgacgccctgctgggcgacccccagtgcgacggcttccagaacaagaaatgggacctgttcgtggaaagaagcaaggcctacagcaactgctacccctacgacgtgcccgactacgccagcctgagaagcctggtggccagcagcggcacactggaattcaacaacgagagcttcaactggaccggcgtgacccagaacggcaccagcagcgcctgcatcagaagaagcaacaacagcttcttcagcagactgaactggctgacccacctgaagttcaagtaccccgccctgaacgtgaccatgcccaacaacgaaaagttcgacaagctgtacatctggggcgtgcaccaccccggcaccgacaacgatcagatcttcccatacgcccaggccagcggcagaatcaccgtgtccaccaagagaagccagcagaccgtgatccccaacatcggcagcagacccagagtccgcaacatccccagccggatcagcatctactggaccatcgtgaagcccggcgacatcctgctgatcaacagcaccggcaacctgatcgcccccagaggctacttcaagattcggagcggcaagagcagcatcatgcggagcgacgcccccatcggcaagtgcaacagcgagtgcatcacccccaacggcagcatccccaacgacaagcccttccagaacgtgaaccggatcacctacggcgcctgccccagatacgtgaagcagaacaccctgaagctggccaccggcatgcggaacgtgcccgagaagcagaccagaggcatcttcggcgccattgccggcttcatcgagaacggctgggagggcatggtggacggttggtacggcttccggcaccagaacagcgagggcattggacaggccgccgacctgaagtctacccaggccgccatcgaccagatcaacggcaagctgaacagactgatcggcaagaccaacgagaagttccaccagatcgagaaagaattcagcgaggtggaaggccggatccaggacctggaaaagtacgtggaagataccaagatcgacctgtggtcctacaacgccgagctgctggtggccctggaaaaccagcacaccatcgacctgaccgacagcgagatgaacaagctgttcgagaaaaccaagaagcagctgcgcgagaacgccgaggacatgggcaacggctgcttcaagatctaccacaagtgcgacaatgcctgcatcggcagcatccggaacggcacctacgaccacaacgtgtacagggacgaggccctgaacaaccggttccagatcaagggcgtggaactgaagtccggctacaaggactggatcctgtggatcagcttcgccatcagctgctttctgctgtgcgtggccctgctgggcttcatcatgtgggcctgccagaaaggcaacatccggtgcaacatctgcatttaaggcgcgcccacccagcggccgc

SEQ ID NO: 16 - protein, 566 aa, Influenza virus
MKTIIALSYILCLVFTQKLPGNDNSTATLCLGHHAVPNGTIVKTITNDQIEVTNATELVQSSSTGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTHLKFKYPALNVTMPNNEKFDKLYIWGVHHPGTDNDQIFPYAQASGRITVSTKRSQQTVIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 17 - DNA, 1784 bp, Influenza virus
  source 1..1784: mol_type = unassigned DNA
gggcccctataactctctacggctaacctgaatggactacgacatagtctagtccgccaagatgaagaccatcattgccctgagctacatcctgtgcctggtgttcgcccagaagctgcccggcaacgacaatagcaccgccaccctgtgtctgggccaccacgccgtgcctaacggcaccatcgtgaaaaccatcaccaacgaccggatcgaagtgaccaacgccaccgagctggtgcagaacagcagcatcggcgagatctgcgacagcccccaccagatcctggacggcgagaactgcaccctgatcgacgccctgctgggcgacccccagtgcgacggcttccagaacaagaaatgggacctgttcgtggaaagaagcaaggcctacagcaactgctacccctacgacgtgcccgactacgccagcctgagaagcctggtggccagcagcggcacactggaattcaacaacgagagcttcaactggaacggcgtgacccagaacggcaccagcagcgcctgcatcagaagaagcaacaacagcttcttcagcagactgaactggctgacccacctgaacttcaagtaccccgccctgaacgtgaccatgcccaacaacgagcagttcgacaagctgtacatctggggcgtgcaccaccccggcaccgacaaggaccagatcttcctgtacgcccagcccagcggcagaatcaccgtgtccaccaagagaagccagcaggccgtgatccccaacatcggcagcagaccccggatccgcaacatccccagccggatcagcatctactggaccatcgtgaagcccggcgacatcctgctgatcaacagcaccggcaacctgatcgcccccagaggctacttcaagattcggagcggcaagagcagcatcatgcggagcgacgcccccatcggcaagtgcaagagcgagtgcatcacccccaacggcagcatccccaacgacaagcccttccagaacgtgaaccggatcacctacggcgcctgccccagatacgtgaagcagagcaccctgaagctggccaccggcatgcggaacgtgcccgagaagcagaccagaggcatcttcggcgccattgccggcttcatcgagaacggctgggagggcatggtggacggttggtacggcttccggcaccagaacagcgagggcagaggacaggccgccgacctgaagtctacccaggccgccatcgaccagatcaacggcaagctgaacagactgatcggcaagaccaacgagaagttccaccagatcgagaaagaattcagcgaggtggaaggccggatccaggacctggaaaagtacgtggaagataccaagatcgacctgtggtcctacaacgccgagctgctggtggccctggaaaaccagcacaccatcgacctgaccgacagcgagatgaacaagctgttcgagaaaaccaagaagcagctgcgcgagaacgccgaggacatgggcaacggctgcttcaagatctaccacaagtgcgacaatgcctgcatcggcagcatccggaacggcacctacgaccacgacgtgtacagggacgaggccctgaacaaccggttccagatcaagggcgtggaactgaagtccggctacaaggactggatcctgtggatcagcttcgccatcagctgctttctgctgtgcgtggccctgctgggcttcatcatgtgggcctgccagaaaggcaacatccggtgcaacatctgcatttaaggcgcgcccacccagcggccgc

SEQ ID NO: 18 - protein, 566 aa, Influenza virus
MKTIIALSYILCLVFAQKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWNGVTQNGTSSACIRRSNNSFFSRLNWLTHLNFKYPALNVTMPNNEQFDKLYIWGVHHPGTDKDQIFLYAQPSGRITVSTKRSQQAVIPNIGSRPRIRNIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 19 - DNA, 1695 bp, Influenza virus
  source 1..1695: mol_type = unassigned DNA
atggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggcagggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaa

SEQ ID NO: 20 - protein, 564 aa, Influenza virus
MEKIVLLLAIVSLVKSDQICIGYHANNSTEQVDTIMEKNVTVTHAQDILEKTHNGKLCDLDGVKPLILRDCSVAGWLLGNPMCDEFLNVPEWSYIVEKINPANDLCYPGNFNDYEELKHLLSRINHFEKIQIIPKSSWSDHEASAGVSSACPYQGRSSFFRNVVWLIKKDNAYPTIKRSYNNTNQEDLLVLWGIHHPNDAAEQTRLYQNPTTYISVGTSTLNQRLVPKIATRSKVNGQSGRMEFFWTILKPNDAINFESNGNFIAPENAYKIVKKGDSTIMKSELEYGNCNTKCQTPIGAINSSMPFHNIHPLTIGECPKYVKSSRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQILSIYSTVASSLALAIMVAGLSLWMCSNGSLQCRICI

SEQ ID NO: 21 - DNA, 1516 bp, Influenza virus
  source 1..1516: mol_type = unassigned DNA
atgaacactcaaatcctggtattcgctctgattgcgatcattccaacaaatgcagacaaaatctgcctcggacatcatgccgtgtcaaacggaaccaaagtaaacacattaactgaaagaggagtggaagtcgtcaatgcaactgaaacagtggaacgaacaaacatccccaggatctgctcaaaagggaaaaggacagttgacctcggtcaatgtggactcctggggacaatcactggaccacctcaatgtgaccaattcctagaattttcagccgatttaattattgagaggcgagaaggaagtgatgtctgttatcctgggaaattcgtgaatgaagaagctctgaggcaaattctcagagaatcaggcggaattgacaaggaagcaatgggattcacatacagtggaataagaactaatggagcaaccagtgcatgtaggagatcaggatcttcattctatgcagaaatgaaatggctcctgtcaaacacagataatgctgcattcccgcagatgactaagtcatataaaaatacaagaaaaagcccagctctaatagtatgggggatccatcattccgtatcaactgcagagcaaaccaagctatatgggagtggaaacaaactggtgacagttgggagttctaattatcaacaatcttttgtaccgagtccaggagcgagaccacaagttaatggtctatctggaagaattgactttcattggctaatgctaaatcccaatgatacagtcactttcagtttcaatggggctttcatagctccagaccgtgcaagcttcctgagaggaaaatctatgggaatccagagtggagtacaggttgatgccaattgtgaaggggactgctatcatagtggagggacaataataagtaacttgccatttcagaacatagatagcagggcagttggaaaatgtccgagatatgttaagcaaaggagtctgctgctagcaacagggatgaagaatgttcctgagattccaaaaggaagaggcctatttggtgctatagcgggtttcattgaaaatggatgggaaggcctaattgatggttggtatggtttcagacaccagaatgcacagggagagggaactgctgcagattacaaaagcactcaatcggcaattgatcaaataacaggaaaattaaaccggcttatagaaaaaaccaaccaacaatttgagttgatagacaatgaattcaatgaggtagagaagcaaatcggtaatgtgataaattggaccagagattctataacagaagtgtggtcatacaatgctgaactcttggtagcaatggagaaccagcatacaattgatctggctgattcagaaatggacaaactgtacgaacgagtgaaaagacagctgagagagaatgctgaagaagatggcactggttgctttgaaatatttcacaagtgtgatgatgactgtatggccagtattagaaataacacctatgatcacagcaaatacagggaagaggcaa

SEQ ID NO: 22 - protein, 506 aa, Influenza virus
  VARIANT 506: note = misc_feature - Xaa can be any naturally occurring amino acid
MNTQILVFALIAIIPTNADKICLGHHAVSNGTKVNTLTERGVEVVNATETVERTNIPRICSKGKRTVDLGQCGLLGTITGPPQCDQFLEFSADLIIERREGSDVCYPGKFVNEEALRQILRESGGIDKEAMGFTYSGIRTNGATSACRRSGSSFYAEMKWLLSNTDNAAFPQMTKSYKNTRKSPALIVWGIHHSVSTAEQTKLYGSGNKLVTVGSSNYQQSFVPSPGARPQVNGLSGRIDFHWLMLNPNDTVTFSFNGAFIAPDRASFLRGKSMGIQSGVQVDANCEGDCYHSGGTIISNLPFQNIDSRAVGKCPRYVKQRSLLLATGMKNVPEIPKGRGLFGAIAGFIENGWEGLIDGWYGFRHQNAQGEGTAADYKSTQSAIDQITGKLNRLIEKTNQQFELIDNEFNEVEKQIGNVINWTRDSITEVWSYNAELLVAMENQHTIDLADSEMDKLYERVKRQLRENAEEDGTGCFEIFHKCDDDCMASIRNNTYDHSKYREEAX

SEQ ID NO: 23 - DNA, 41 bp, synthetic construct
  misc_feature 1..41: note = forward primer for influenza H1N1 A/California/07/2009 insert
  source 1..41: mol_type = other DNA
attcccgtcgacgccaccatgaaggcaatactagtagttct

SEQ ID NO: 24 - DNA, 44 bp, synthetic construct
  misc_feature 1..44: note = reverse primer for influenza H1N1 A/California/07/2009 insert
  source 1..44: mol_type = other DNA
atttacgcctaggttatcaaatacatattctacactgtagagac

SEQ ID NO: 25 - DNA, 43 bp, synthetic construct
  misc_feature 1..43: note = forward primer for H7 HA gene from A/Shanghai/2/2013
  source 1..43: mol_type = other DNA
aattaagtcgacgccaccatgaacactcaaatcctggtattcg

SEQ ID NO: 26 - DNA, 40 bp, synthetic construct
  misc_feature 1..40: note = reverse primer for H7 HA gene from A/Shanghai/2/2013
  source 1..40: mol_type = other DNA
aattaatctagattatcatatacaaatagtgcaccgcatg

SEQ ID NO: 27 - protein, 15 aa, synthetic construct
  REGION 1..15: note = 27 peptide
ALNNRFQIKGVELKS

SEQ ID NO: 28 - DNA, 47 bp, synthetic construct
  misc_feature 1..47: note = SB63 primer
  source 1..47: mol_type = other DNA
catagtctagtcgacgccaccatggagaaaatagtgcttcttcttgc

SEQ ID NO: 29 - DNA, 95 bp, synthetic construct
  misc_feature 1..95: note = SB74 primer
  source 1..95: mol_type = other DNA
gtcgaagttcagggtctgcttcacgggggccacgatcttctgcttgtgccgggcctcccgcttggcccgaatgcaaattctgcattgtaacgatc

SEQ ID NO: 30 - DNA, 91 bp, synthetic construct
  misc_feature 1..91: note = SB76 primer
  source 1..91: mol_type = other DNA
gtgaagcagaccctgaacttcgacctgctgaagctggccggcgacgtggagagcaaccccggccccatgaaggcaatactagtagttctgc

SEQ ID NO: 31 - DNA, 51 bp, synthetic construct
  misc_feature 1..51: note = SB66 primer
  source 1..51: mol_type = other DNA
ggcgtagcggcggccgcttatcaaatacatattctacactgtagagaccca